(19) United States
(12) Patent Application Publication
(10) Pub. No.: US 2024/0279716 A1
(43) Pub. Date: Aug. 22, 2024

(54) DETECTION OF MULTIPLE TARGET NUCLEIC ACID USING MULTIPLE DETECTION TEMPERATURES

(71) Applicant: SEEGENE, INC., Seoul (KR)

(72) Inventors: Han Bit LEE, Seoul (KR); Jeong Woo KIM, Seoul (KR); Hyeon Be KIM, Seoul (KR)

(73) Assignee: SEEGENE, INC., Seoul (KR)

(21) Appl. No.: 18/570,381

(22) PCT Filed: Jun. 17, 2022
(86) PCT No.: PCT/KR2022/008659

§ 371 Date: Dec. 14, 2023

(30) Foreign Application Priority Data
Jun. 17, 2021 (KR) 10-2021-0078463

(51) Int. Cl. C12Q 1/6818; C12Q 1/6851

(52) U.S. Cl. CPC C12Q 1/6818; C12Q 1/6851

(57) ABSTRACT

The present disclosure pertains to a method for detecting multiple target nucleic acids by a single type of label alone in a single reaction vessel by using multiple detection temperatures, which is characterized by providing a signal change dependent on the presence of a corresponding target nucleic acid at a corresponding detection temperature of each of the target nucleic acids. The conventional techniques using a single type of label is subjected to melting analysis after target amplification so as to detect multiple target nucleic acids. In contrast, the present method does not require melting curve analysis after target amplification, even using a single type of label, and thus can remarkably reduce the analysis time.

### TECHNICAL FIELD

The present invention relates to a method of detecting multiple target nucleic acids, using multiple detection temperatures.

### BACKGROUND ART

For detection of target nucleic acids, real-time detection methods capable of detecting target nucleic acids with monitoring target amplification in a real-time manner are widely used. The real-time detection methods generally use labeled probes or primers specifically hybridized with target nucleic acids.

Examples of methods using hybridization between labeled probes and target nucleic acids include Molecular beacon method using dual-labeled probes with hairpin structure (Tyagi et al., Nature Biotechnology v. 14 Mar. 1996), HyBeacon method (French D J et al., Mol. Cell Probes, 15(6):363-374 (2001)), Hybridization probe method using two probes, each labeled as donor and acceptor (Bernard et al., 147-148 Clin. Chem. 2000; 46) and Lux method using single-labeled oligonucleotides (U.S. Pat. No. 7,537,886). TaqMan method (U.S. Pat. Nos. 5,210,015 and 5,538,848) using cleavage of dual-labeled probe by 5′-nuclease activity of DNA polymerase is also widely used in the present technical field.

Examples of methods using labeled primers include Sunrise primer method (Nazarenko et al., 2516-2521 Nucleic Acids Research, 1997, v. 25 no. 12, and U.S. Pat. No. 6,117,635), Scorpion primer method (Whitcombe et al., 804-807, Nature Biotechnology v. 17 Aug. 1999 and U.S. Pat. No. 6,326,145), and TSG primer method (WO 2011/078441).

As alternative approaches, real-time detection methods using duplexes formed depending on the presence of target nucleic acids have been proposed: Invader assay (U.S. Pat. Nos. 5,691,142, 6,358,691 and 6,194,149), PTOCE (PTO cleavage and extension) method (WO 2012/096523), PCE-SH (PTO Cleavage and Extension-Dependent Signaling Oligonucleotide Hybridization) method (WO 2013/115442), and PCE-NH (PTO Cleavage and Extension-Dependent Non-Hybridization) method (PCT/KR2013/012312).

Since the above-described conventional real-time detection manners can only detect a single target nucleic acid with a single label, the number of target nucleic acids that can be simultaneously detected in a single reaction is limited by the number of labels that can be used (e.g., 5 or less).

Although melting analysis may be used to detect multiple target nucleic acids with the use of a single label, it has a disadvantage of a longer performance time than the real-time manners.

Meanwhile, in recent years, methods that can detect multiple target nucleic acids in real time with a single type of a label, using signal detection at different detection temperatures, have been proposed (WO 2015/147370, WO 2015/147377, WO 2015/147382, WO 2015/147412). However, among such different detection temperatures, at a relatively lower detection temperature, these methods also allow target nucleic acids having a relatively higher detection temperature to be detected along with target nucleic acids having the relatively lower detection temperature, thus necessitating the process of obtaining difference between signals detected at different detection temperatures (e.g., difference between a signal detected at a relatively lower detection temperature and a signal detected at a relatively higher detection temperature).

Accordingly, there is a need to develop a novel method or approach for detection of multiple target nucleic acids that has highly improved convenience and high efficiency.

Throughout the present application, various patents and publications are referenced and citations are provided in parentheses. The disclosure of these patents and publications in their entirety are hereby incorporated by references into the present disclosure in order to more fully describe the present invention and the state of the art to which the present invention pertains.

### DISCLOSURE OF INVENTION

### Technical Problem

The present inventors have endeavored to develop a method for detecting multiple target nucleic acids by using a single type of label and a single type of detector in a single reaction vessel. As a result, the present inventors have confirmed that, while using multiple detection temperatures, by adjusting the detection temperatures and the signal generation mechanisms so that only a single signal indicative of the presence of a single target nucleic acid sequence is provided at a single detection temperature, it is possible to detect multiple target nucleic acids in a real-time manner by using a single type of label and a single type of detector in a single reaction vessel, with improved convenience and high cost-effectiveness and efficiency.

Therefore, it is an object of the present disclosure to provide a method for detecting n target nucleic acids in a sample.

Another object of the present disclosure is to provide a kit for detecting n target nucleic acids in a sample.

### Solution to Problem

According to one aspect of the present disclosure, provided is a method for detecting n target nucleic acids in a sample, comprising: (a) detecting signals at n detection temperatures, while incubating with n compositions for detecting the target nucleic acids, a sample suspected of containing at least one of the n target nucleic acids in a reaction vessel; wherein n is an integer of 2 or more, wherein the incubation comprises a plurality of cycles and the detection of signals is carried out at at least one of the plurality of cycles, wherein each of the n compositions for detecting the target nucleic acids provides a signal change at a corresponding detection temperature among the n detection temperatures in the presence of a corresponding target nucleic acid, the signal change indicating the presence of a corresponding target nucleic acid, wherein a composition for detecting an i^{th} target nucleic acid among the n compositions for detecting the target nucleic acids provides a signal change at an i^{th} detection temperature among the n detection temperatures and provides a constant signal at the other detection temperatures in the presence of the i^{th} target nucleic acid, the signal change indicating the presence of the i^{th} target nucleic acid, and wherein i represents an integer from 1 to n, and the i^{th} detection temperature is lower than the (i+1)^{th} detection temperature; and (b) determining the presence of the n target nucleic acids from the signals detected in step (a), wherein the presence of the i^{th} target nucleic acid is determined by the signal change detected at the i^{th} detection temperature.

According to an embodiment of the present disclosure, in the temperature range covering all of the n detection temperatures, the composition for detecting the i^{th} target nucleic acid has a signal-changing temperature range (SChTR) in which the signal changes depending on the presence of the i^{th} target nucleic acid, and a signal-constant temperature range (SCoTR) in which the signal is constant even in the presence of the i^{th} target nucleic acid.

According to an embodiment of the present disclosure, the composition for detecting the i^{th} target nucleic acid has one or two signal-constant temperature ranges.

According to an embodiment of the present disclosure, the composition for detecting the i^{th} target nucleic acid is any one of: (i) an Under-Signal-Change (UnderSC) composition having a characteristic that the signal-changing temperature range is lower than the signal-constant temperature range; (ii) an Over-Signal-Change (OverSC) composition having a characteristic that the signal-changing temperature range is higher than the signal-constant temperature range; and (iii) an Inter-Signal-Change (InterSC) composition having a characteristic that the signal-changing temperature range is higher than one of two signal-constant temperature ranges, and lower than the other of the two signal-constant temperature ranges.

According to an embodiment of the present disclosure, the i^{th} detection temperature is selected within the signal-changing temperature range of the composition for detecting the i^{th} target nucleic acid, wherein the i^{th} detection temperature is not included in the signal-changing temperature ranges of the compositions for detecting the other target nucleic acids.

According to an embodiment of the present disclosure, the signal-changing temperature range of the composition for detecting the i^{th} target nucleic acid overlaps partially with the signal-changing temperature range of a composition for detecting a target nucleic acid having an adjacent detection temperature, and does not overlap with the signal-changing temperature range of a composition for detecting a target nucleic acid having a detection temperature that is not adjacent thereto.

According to an embodiment of the present disclosure, when n is 2, the composition for detecting the first target nucleic acid is an UnderSC composition or an InterSC composition, and the composition for detecting the second target nucleic acid is an InterSC composition or an OverSC composition.

According to an embodiment of the present disclosure, when n is 3 or more, the composition for detecting the first target nucleic acid is an UnderSC composition or an InterSC composition, the composition for detecting the n^{th} target nucleic acid is an InterSC composition or an OverSC composition, and each of compositions for detecting target nucleic acids other than the first target nucleic acid and the n^{th} target nucleic acid is an InterSC composition.

According to an embodiment of the present disclosure, the composition for detecting the i^{th} target nucleic acid comprises a label that provides a signal dependent on the presence of the i^{th} target nucleic acid.

According to an embodiment of the present disclosure, the label is linked to an oligonucleotide or is incorporated into an oligonucleotide during the incubation.

According to an embodiment of the present disclosure, the composition for detecting the i^{th} target nucleic acid provides a duplex providing a signal change.

According to an embodiment of the present disclosure, the composition for detecting the i^{th} target nucleic acid provides a duplex providing a signal change, and the composition for detecting the i^{th} target nucleic acid provides a signal from the label when the duplex providing the signal change is in an associated form.

According to an embodiment of the present disclosure, the composition for detecting the i^{th} target nucleic acid provides a duplex providing a signal change, and the composition for detecting the i^{th} target nucleic acid provides a signal from the label when the duplex providing the signal change is in a dissociated form.

According to an embodiment of the present disclosure, the duplex providing the signal change has initially been included in the composition for detecting the i^{th} target nucleic acid.

According to an embodiment of the present disclosure, the duplex providing the signal change is generated by hybridization between a label-linked oligonucleotide and an oligonucleotide hybridizable with the label-linked oligonucleotide.

According to an embodiment of the present disclosure, the duplex providing the signal change is generated in incubating.

According to an embodiment of the present disclosure, the duplex providing the signal change is generated by hybridization between a label-linked oligonucleotide and the target nucleic acid.

According to an embodiment of the present disclosure, the duplex providing the signal change is generated by a cleavage reaction dependent on the presence of a target nucleic acid.

According to an embodiment of the present disclosure, the composition for detecting the target nucleic acid comprises a tagging oligonucleotide that hybridizes to the target nucleic acid, and the cleavage reaction dependent on the presence of the target nucleic acid involves cleavage of the tagging oligonucleotide.

According to an embodiment of the present disclosure, the duplex providing the signal change is a single-typed duplex or plural-typed duplexes.

According to an embodiment of the present disclosure, when the duplex providing the signal change is the single-typed duplex, the amount of the single-typed duplex changes depending on the presence of the target nucleic acid, thereby changing the signal.

According to an embodiment of the present disclosure, when the duplex providing the signal change is the plural-typed duplexes, the amount ratio between the plural-typed duplexes changes depending on the presence of the target nucleic acid, thereby changing the signal.

According to an embodiment of the present disclosure, when the duplex is the plural-typed duplexes, the Tm values of the duplexes are different from each other.

According to an embodiment of the present disclosure, at least two of the plural-typed duplexes comprise the same single-strand.

According to an embodiment of the present disclosure, the duplex providing the signal change comprises a label.

According to an embodiment of the present disclosure, the composition for detecting the i^{th} target nucleic acid provides a duplex providing a signal change, and the signal-changing temperature range of the composition for detecting the i^{th} target nucleic acid is determined depending on the length and/or sequence of the duplex.

According to an embodiment of the present disclosure, the detection of signals is carried out at at least two of the plurality of cycles.

According to an embodiment of the present disclosure, the signal change is measured using the signals detected at the at least two of the plurality of cycles.

According to an embodiment of the present disclosure, the signal change at the i^{th} detection temperature is measured using a signal detected at the at least one of the plurality of cycles and a reference signal value.

According to an embodiment of the present disclosure, the reference signal value is obtained from a reaction in the absence of the i^{th} target nucleic acid.

According to an embodiment of the present disclosure, the detection of a signal at each of the n detection temperatures is carried out using a single type of detector.

According to an embodiment of the present disclosure, the signals detected at the n detection temperatures are not differentiated from each other by the single type of detector.

According to an embodiment of the present disclosure, the incubation comprises a nucleic acid amplification reaction.

According to an embodiment of the present disclosure, the nucleic acid amplification reaction is a polymerase chain reaction (PCR).

According to another aspect of the present disclosure, provided is a method for detecting two target nucleic acids in a sample, comprising:
(a) detecting signals at a first detection temperature and a second detection temperature, while incubating the sample suspected of containing at least one of the two target nucleic acids with a composition for detecting a first target nucleic acid and a composition for detecting a second target nucleic acid in a reaction vessel; wherein the incubation comprises a plurality of cycles, and the detection of signals is carried out at at least one of the plurality of cycles, wherein the composition for detecting the first target nucleic acid provides a signal change at the first detection temperature and provides a constant signal at the second detection temperature in the presence of the first target nucleic acid, the signal change indicating the presence of the first target nucleic acid; and the composition for detecting the second target nucleic acid provides a signal change at the second detection and provides a constant signal at the first detection temperature in the presence of the second target nucleic acid, the signal change indicating the presence of the second target nucleic acid, and wherein the first detection temperature is lower than the second detection temperature, and (b) determining the presence of the two target nucleic acids from the signals detected in step (a); wherein the presence of the first target nucleic acid is determined by the signal change detected at the first detection temperature, and the presence of the second target nucleic acid is determined by the signal change detected at the second detection temperature.

According to another aspect of the present disclosure, provided is a method for detecting three target nucleic acids in a sample, comprising:
(a) detecting signals at a first detection temperature, a second detection temperature, and a third detection temperature, while incubating the sample suspected of containing at least one of the three target nucleic acids with a composition for detecting a first target nucleic acid, a composition for detecting a second target nucleic acid, and a composition for detecting a third target nucleic acid in a reaction vessel; wherein the incubation comprises a plurality of cycles, and the detection of signals is carried out at at least one of the plurality of cycles, wherein the composition for detecting the first target nucleic acid provides a signal change at the first detection temperature and provides a constant signal at the second detection temperature and the third detection temperature in the presence of the first target nucleic acid, the signal change indicating the presence of the first target nucleic acid; the composition for detecting the second target nucleic acid provides a signal change at the second detection temperature and provides a constant signal at the first detection temperature and the third detection temperature in the presence of the second target nucleic acid, the signal change indicating the presence of the second target nucleic acid; and the composition for detecting the third target nucleic acid provides a signal change at the third detection temperature and provides a constant signal at the first detection temperature and the second detection temperature in the presence of the third target nucleic acid, the signal change indicating the presence of the third target nucleic acid, and wherein the first detection temperature is lower than the second detection temperature, and the second detection temperature is lower than the third detection temperature, and (b) determining the presence of the three target nucleic acids from the signals detected in step (a), wherein the presence of the first target nucleic acid is determined by the signal change detected at the first detection temperature, the presence of the second target nucleic acid is determined by the signal change detected at the second detection temperature, and the presence of the third target nucleic acid is determined by the signal change detected at the third detection temperature.

According to another aspect of the present disclosure, provided is a kit, comprising n compositions for detecting n target nucleic acids in a sample, wherein n is an integer of 2 or more, wherein each of the n compositions for detecting the n target nucleic acids provides a signal change at a corresponding detection temperature among n detection temperatures, the signal change indicating the presence of a corresponding target nucleic acid, wherein a composition for detecting an i^{th} target nucleic acid among the n target nucleic acids provides a signal change at an i^{th} detection temperature among the n detection temperatures and provides a constant signal at the other detection temperatures in the presence of the i^{th} target nucleic acid, and wherein i represents an integer from 1 to n, and the i^{th} detection temperature is lower than the (i+1)^{th} detection temperature.

### Advantageous Effects of Invention

The features and advantages of the present disclosure will be summarized as follows:

(a) The present disclosure pertains to a method for detecting multiple target nucleic acids by a single type of label alone in a single reaction vessel by using multiple detection temperatures, which is characterized by providing a signal change dependent on the presence of a corresponding target nucleic acid at a corresponding detection temperature of each of the target nucleic acids.

(b) Interestingly, the present inventors have found that various signal generation mechanisms for detection of target nucleic acids well known in the art involve a temperature range in which the signal changes depending on the presence of the target nucleic acid (that is, signal-changing temperature range) and a temperature range in which the signal does not change even in the presence of the target nucleic acid (that is, signal-constant temperature range), and can be categorized into three types according to the number and/or order of these signal-changing temperature range and signal-constant temperature range. The present inventors have also found that an appropriate combination of these three types of signal generation mechanisms is useful in detection of multiple target nucleic acids.

(c) In particular, when the signal generation mechanism involving one signal-changing temperature range and two signal-constant temperature ranges (that is, the signal generation mechanism adoptable by the InterSC composition for detecting a target nucleic acid) is applied to the present disclosure, the number of target nucleic acids detectable by the method according to the present disclosure further increased.

(d) By adopting one or more of the above-described three types of signal generation mechanisms, and by adjusting the signal-changing temperature ranges of the one or more signal generation mechanisms such that only the signal indicating the presence of a single target nucleic acid is provided at each detection temperature, the present disclosure has advantages in that the presence of a particular target nucleic acid can be determined by a signal change measured at a particular detection temperature alone, with no need to consider signal changes at the other detection temperatures (e.g., detection temperatures other than the particular detection temperature, that is, detection temperatures showing signal changes indicating the presence of other target nucleic acids). According to the present disclosure, the method according to the present disclosure utilizes n compositions for detecting n different target nucleic acids, each composition corresponding to each of the target nucleic acids. In one embodiment, each of the n compositions for detecting target nucleic acids adopts one of the three types of signal generation mechanisms such that a signal change indicating the presence of a corresponding target nucleic acid is provided only at a corresponding detection temperature among the n detection temperatures.

(e) The method according to the present disclosure allows to detect multiple target nucleic acids in real-time, by using a single type of label alone in a single reaction vessel. The conventional techniques using a single type of label is subjected to melting analysis after target amplification so as to detect multiple target nucleic acids. In contrast, the present method does not require melting curve analysis after target amplification, even using a single type of label, and thus can remarkably reduce the analysis time.

### BRIEF DESCRIPTION OF DRAWINGS

FIGS. 1a to 1h show examples of signal generation mechanisms adoptable by an UnderSC composition. In each drawing, “(a)” represents pre-incubation in the presence or absence of the target nucleic acid or post-incubation in the absence of the target nucleic acid, and “(b)” represents post-incubation in the presence of the target nucleic acid.

FIGS. 2a to 2c show examples of signal generation mechanisms adoptable by an OverSC composition. In each drawing, “(a)” represents pre-incubation in the presence or absence of the target nucleic acid or post-incubation in the absence of the target nucleic acid, and “(b)” represents post-incubation in the presence of the target nucleic acid.

FIGS. 3a to 3b show examples of signal generation mechanisms adoptable by an InterSC composition. In each drawing, “(a)” represents pre-incubation in the presence or absence of the target nucleic acid or post-incubation in the absence of the target nucleic acid, and “(b)” represents post-incubation in the presence of the target nucleic acid.

FIG. 4 shows temperature ranges each selectable as a detection temperature in the case that SChTRs do not overlap each other or that SChTRs overlap partially each other.

FIG. 5 schematically shows the signal generation mechanism of PTOCE-based-1, which is adoptable by an UnderSC composition used in Examples.

As depicted in the drawing, CTO has a reporter molecule and a quencher molecule at its templating portion.

FIG. 6 schematically shows the signal generation mechanism of PTOCE-based-2, which is adoptable by an InterSC composition used in Examples.

As depicted in the drawing, PTO has a quencher molecule at its 5′-tagging portion, and CTO has a reporter molecule at its capturing portion.

FIG. 7 schematically shows the signal generation mechanism of dual-quenching method, which is adoptable by an OverSC composition used in Examples.

As depicted in the drawing, PTO has a first quencher molecule and a reporter molecule and CQO has a second quencher molecule.

FIG. 8 schematically shows the signal generation mechanisms of two compositions, an UnderSC composition and an InterSC composition, used in Combination 1 in Examples.

FIG. 9 schematically shows the signal generation mechanisms of two compositions, an UnderSC composition and an OverSC composition, used in Combination 2 in Examples.

FIG. 10 schematically shows the signal generation mechanisms of two compositions, an InterSC composition and an InterSC composition, used in Combination 3 in Examples.

FIG. 11 schematically shows the signal generation mechanisms of two compositions, an InterSC composition and an OverSC composition, used in Combination 4 in Examples.

FIG. 12 schematically shows the signal generation mechanisms of three compositions, an UnderSC composition, an InterSC composition and an OverSC composition, used in Combination 5 in Examples.

FIG. 13 shows the results of real-time PCR for Combination 1 in Examples.

In the drawing, “Target 1” represents genomic DNA of Chlamydia trachomatis (CT), “Target 2” represents Neisseria gonorrhoeae (NG), “NTC” represents No Template Control, and “RFU” represents Relative Fluorescence Units.

FIG. 14 shows the results of real-time PCR for Combination 2 in Examples.

In the drawing, “Target 1” represents genomic DNA of Chlamydia trachomatis (CT), “Target 2” represents Ureaplasma parvum (UP), “NTC” represents No Template Control, and “RFU” represents Relative Fluorescence Units.

FIG. 15 shows the results of real-time PCR for Combination 3 in Examples.

In the drawing, “Target 1” represents genomic DNA of Chlamydia trachomatis (CT), “Target 2” represents Neisseria gonorrhoeae (NG), “NTC” represents No Template Control, and “RFU” represents Relative Fluorescence Units. FIG. 16 shows the results of real-time PCR for Combination 4 in Examples.

In the drawing, “Target 1” represents genomic DNA of Neisseria gonorrhoeae (NG), “Target 2” represents Ureaplasma parvum (UP), “NTC” represents No Template Control, and “RFU” represents Relative Fluorescence Units.

FIGS. 17a and 17b show the results of real-time PCR for Combination 5 in Examples.

In each drawing, “Target 1” represents genomic DNA of Chlamydia trachomatis (CT), “Target 2” represents Neisseria gonorrhoeae (NG), “Target 3” represents Ureaplasma parvum (UP), “NTC” represents No Template Control, and “RFU” represents Relative Fluorescence Units.

### BEST MODE FOR CARRYING OUT THE INVENTION

The present inventors have found that various signal generation mechanisms for detection of a target nucleic acid well known in the art involve a temperature range in which in signal changes depending on the presence of the target nucleic acid (that is, a signal-changing temperature range) and a temperature range in which there is no signal change even in the presence of the target nucleic acid (that is, signal-constant temperature range), and can be categorized into three types according to the number and/or order of these signal-changing temperature range and signal-constant temperature range. By applying various combinations of these three types of signal generation mechanisms to detection of target nucleic acids, the present inventors have developed a novel method for detecting multiple target nucleic acids by using a single type of label and a single type of detector in a single reaction vessel.

### I. The Process for Detecting Target Nucleic Acids

According to one aspect of the present disclosure, provided is a method for detecting n target nucleic acids in a sample, comprising: (a) detecting signals at n detection temperatures, while incubating with n compositions for detecting the target nucleic acids, a sample suspected of containing at least one of the n target nucleic acids in a reaction vessel; wherein n is an integer of 2 or more, wherein the incubation comprises a plurality of cycles and the detection of signals is carried out at at least one of the plurality of cycles, wherein each of the n compositions for detecting the target nucleic acids provides a signal change at a corresponding detection temperature among the n detection temperatures in the presence of a corresponding target nucleic acid, the signal change indicating the presence of a corresponding target nucleic acid, wherein a composition for detecting an i^{th} target nucleic acid among the n compositions for detecting the target nucleic acids provides a signal change at an i^{th} detection temperature among the n detection temperatures and provides a constant signal at the other detection temperatures in the presence of the i^{th} target nucleic acid, the signal change indicating the presence of the i^{th} target nucleic acid, and wherein i represents an integer from 1 to n, and the i^{th} detection temperature is lower than the (i+1)^{th} detection temperature; and (b) determining the presence of the n target nucleic acids from the signals detected in step (a), wherein the presence of the i^{th} target nucleic acid is determined by the signal change detected at the i^{th} detection temperature.

The present invention may be described for each step as follows:

### Step (a): Incubation and Detection of Signals

First, in a single reaction vessel, a sample suspected of containing at least one of the n target nucleic acids is mixed and incubated with n compositions for detecting target nucleic acids.

In one embodiment, n is an integer of 2 or more. For example, n may be 2, 3, 4, 5, 6, 7, 8, 9, 10, 11, 12, 13, 14, 15, 20, 25, 30, 35, 40, 45, or 50, but is not limited thereto.

The term “target nucleic acid”, “target nucleic acid sequence”, or “target sequence” as used herein refers to a nucleic acid sequence to be detected or quantified.

The target nucleic acid sequence includes a single-strand as well as a double-strand. The target nucleic acid sequence includes, not only a sequence newly generated in a reaction, but also a sequence initially present in a nucleic acid sample.

The target nucleic acid includes any DNA (gDNA and cDNA) and RNA molecules, and their hybrids (chimeric nucleic acids). The sequence may be in a double-stranded or single-stranded form.

Target nucleic acids include any naturally occurring procaryotic, eukaryotic (for example, protozoans and parasites, fungi, yeast, higher plants, lower animals, and higher animals, including mammals and humans) or viral (for example, Herpes virus, HIV, influenza virus, Epstein-Barr virus, hepatitis virus, polio virus, etc.) or viroid nucleic acid. In addition, the nucleic acid molecule may be any nucleic acid molecule which is produced or can be produced by recombination, or any nucleic acid molecule which is or can be chemically synthesized. As such, the nucleic acid sequence may or may not be found in nature. The target nucleic acid sequence may be of a known or unknown sequence.

In one embodiment, the n target nucleic acids may include a nucleotide variation. For example, one of the n target nucleic acids may include one type of nucleotide variation, and another one of the n target nucleic acids may include a different type of nucleotide variation.

As used herein, the term “nucleotide variation” refers to any single or multiple nucleotide substitutions, deletions or insertions in a DNA sequence at a particular location among contiguous DNA segments. Such contiguous DNA segments include a gene or any other portion of a chromosome. These nucleotide variations may be mutant or polymorphic allele variations. For example, the nucleotide variation detected in the present disclosure includes single nucleotide polymorphism (SNP), mutation, deletion, insertion, substitution and translocation. Examples of nucleotide variation include numerous variations in a human genome (e.g., variations in the MTHFR (methylenetetrahydrofolate reductase) gene), variations involved in drug resistance of pathogens and tumorigenesis-causing variations. As used herein, the term “nucleotide variation” includes any variation at a particular location in a nucleic acid sequence. That is, the term “nucleotide variation” includes a wild type and any mutation thereof, at a particular location in a nucleic acid sequence.

As used herein, the term “sample” refers to cells, tissues or fluid from a biological source, or any other medium that may be proved to be useful in the present invention, and includes virus, bacteria, tissues, cells, blood, serum, plasma, lymph, milk, urine, feces, intraocular fluid, saliva, semen, brain extract, spinal fluid, appendix, spleen and tonsil tissue extracts, amniotic fluid, ascites, and non-biological samples (e.g., food and water). In addition, the sample includes naturally-occurring nucleic acid molecule isolated from a biological source, and synthesized nucleic acid molecules.

The present invention is used to determine whether at least one of n target nucleic acids is present in a sample. For example, when n is 2, the present invention may be used to determine whether at least one of a first target nucleic acid and a second target nucleic acid is present in a sample. As another example, when n is 3, the present invention may be used to determine whether at least one among a first target nucleic acid, a second target nucleic acid, and a third target nucleic acid is present in a sample.

In one embodiment, the incubation reaction refers to any reaction that induces a signal change depending on the presence of a corresponding target nucleic acid at a corresponding detection temperature, as each of the target nucleic acids reacts with a corresponding composition for detecting a target nucleic acid.

In one embodiment, the incubation includes a plurality of cycles.

In one embodiment, the incubation in step (a) may include an amplification reaction, and may include, for example, a signal amplification reaction and/or a nucleic acid amplification reaction.

In one embodiment, the amplification reaction includes a plurality of cycles.

In one embodiment, the incubation in step (a) is carried out under conditions that allow target amplification and a signal change by a composition for detecting a target nucleic acid. Such conditions include a temperature, salt concentration and pH of a solution.

In one embodiment, the incubation in step (a) is carried out at a signal amplification process with no nucleic acid amplification.

In one embodiment, the signal may be amplified simultaneously with target amplification. Alternatively, the signal may be amplified without target amplification.

In one embodiment, the signal change takes place during a process including signal amplification and target amplification.

In one embodiment, the amplification of the target nucleic acid may be carried out by a polymerase chain reaction (PCR). The PCR is widely used in the art to amplify target nucleic acids and includes cycles of denaturation of target nucleic acids, annealing (hybridization) between target nucleic acids and primers, and extension of primers (Mullis et al., U.S. Pat. Nos. 4,683,195, 4,683,202, and 4,800,159; Saiki et al., (1985) Science 230, 1350-1354).

In one embodiment, the amplification of target nucleic acids may be carried out by ligase chain reaction (LCR) (U.S. Pat. Nos. 4,683,195 and 4,683,202; PCR Protocols: A Guide to Methods and Applications (Innis et al., eds, 1990)), strand displacement amplification (SDA) (Walker, et al., Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), helicase dependent amplification (HAD) (M. Vincent, Y. Xu and H. Kong, EMBO Rep., 2004, 5, 795-800), nucleic acid sequence-based amplification (NASBA) (Compton, Nature 350(6313):91-2 (1991)), rolling circle amplification (RCA) (Lisby, Mol. Biotechnol. 12(1):75-99 (1999); Hatch et al., Genet. Anal. 15(2):35-40 (1999)), Q-Beta Replicase (Lizardi et al., BiolTechnology 6:1197 (1988)), Loop-mediated isothermal amplification (LAMP) (Y. Mori, H. Kanda and T. Notomi, J. Infect. Chemother., 2013, 19, 404-411), or Recombinase Polymerase Amplification (RPA) (J. Li, J. Macdonald and F. von Stetten, Analyst, 2018, 144, 31-67)

Various DNA polymerase may be used in the amplification reaction and include the “Klenow” fragment of E. coli DNA polymerase I, thermostable DNA polymerase, and bacteriophage T7 DNA polymerase. In particular, the polymerase is a thermostable DNA polymerase obtainable from various bacteria, which include Thermus aquaticus (Taq), Thermus thermophilus (Tth), Thermus filiformis, Thermis flavus, Thermococcus literalis, and Pyrococcus furiosus (Pfu). Most of the above polymerases can be isolated as is from bacteria, or can be commercially purchased.

The above-described amplification method can amplify target nucleic acid and/or signal through a repetition of a series of reactions with or without change in temperature. The unit of amplification including the repetition of such series of reactions, is referred to as a “cycle”. The cycle may be expressed as the number of repetitions or a duration, depending on the amplification method used.

In one embodiment, the series of reactions may be carried out sequentially. For example, for a PCR, after denaturation of target nucleic acids (that is, templates), annealing of primers and subsequently, extension of the primers may be carried out sequentially. In this case, the cycle may be expressed as the number of repetitions.

In one embodiment, the series of reactions may be carried out simultaneously. For example, in the LAMP, which is an isothermal amplification assay, annealing of the primers may be taking place on some templates among a plurality of templates, while on some other templates, the primers may have already annealed that extension of the primers may be taking place. In this case, the cycle may be expressed as a duration. In particular, 1 cycle may be 5 seconds, 10 seconds, 1 minute, 2 minutes, 3 minutes, 4 minutes, 5 minutes, 10 minutes, 20 minutes, 30 minutes, 1 hour, or 2 hours.

In one embodiment, the incubation may be carried out for a plurality of cycles that allows for the measurement of a signal change dependent on the presence of a target nucleic acid. For example, the plurality of cycles may include 2 to 100 cycles, 2 to 90 cycles, 2 to 80 cycles, 2 to 70 cycles, 2 to 60 cycles, 2 to 50 cycles, 2 to 40 cycles, 2 to 30 cycles, 2 to 20 cycles, 2 to 10 cycles, 5 to 100 cycles, 5 to 90 cycles, 5 to 80 cycles, 5 to 70 cycles, 5 to 60 cycles, 5 to 50 cycles, 5 to 40 cycles, 5 to 30 cycles, 5 to 20 cycles, 5 to 10 cycles, 10 to 100 cycles, 10 to 90 cycles, 10 to 80 cycles, 10 to 70 cycles, 10 to 60 cycles, 10 to 50 cycles, 10 to 40 cycles, 10 to 30 cycles, 10 to 20 cycles, 20 to 100 cycles, 20 to 90 cycles, 20 to 80 cycles, 20 to 70 cycles, 20 to 60 cycles, 20 to 50 cycles, 20 to 40 cycles, or 20 to 30 cycles, and particularly, may include 10 cycles, 15 cycles, 20 cycles, 25 cycles, 30 cycles, 35 cycles, 40 cycles, 45 cycles, or 50 cycles.

In one embodiment, the detection of signals may be carried out at each cycle, a selected few cycles, or at the end-point, of an incubation reaction including a plurality of cycles.

In one embodiment, the amplification reaction of a target nucleic acid may be a multiple target nucleic acid amplification reaction.

The term “multiple target nucleic acid amplification reaction” used herein refers to a reaction that amplifies two or more nucleic acids as targets in a single reaction vessel. The multiple target nucleic acid amplification reaction refers to a reaction that amplifies two or more nucleic acids together. For example, the multiple target nucleic acid amplification reaction may amplify, in a single reaction, 2 or more, 3 or more, 4 or more, 5 or more, 6 or more, 7 or more, 8 or more, 9 or more, 10 or more, 20 or more, 30 or more, 40 or more, or 50 or more of target nucleic acids together.

In one embodiment, the method of the present invention may detect 2 to 50, 2 to 40, 2 to 30, 2 to 20, 2 to 15, 2 to 12, 2 to 10, 2 to 9, 2 to 8, 2 to 7, 2 to 6, 2 to 5, 3 to 50, 3 to 40, 3 to 30, 3 to 20, 3 to 15, 3 to 12, 3 to 10, 3 to 9, 3 to 8, 3 to 7, 3 to 6, 3 to 5, 4 to 50, 4 to 40, 4 to 30, 4 to 20, 4 to 15, 4 to 12, 4 to 10, 4 to 9, 4 to 8, 4 to 7, 4 to 6 or 4 to 5 target nucleic acids by using a single type of label in a single reaction vessel.

In one embodiment, the method of the present invention, when using multiple types of labels, may detect a greater number of target nucleic acids than the number of target nucleic acids detectable using a single type of label according to the method of the present invention, and for example, may be able to detect more target nucleic acids by a fold increase of the number of label types used.

According to the present invention, the method according to the present disclosure utilizes n different compositions for detecting n target nucleic acids, each of which corresponds to their respective target nucleic acid.

For example, when n is 2, the composition for detecting the first target nucleic acid and the composition for detecting the second target nucleic acid are used; when n is 3, the composition for detecting the first target nucleic acid, the composition for detecting the second target nucleic acid, and the composition for detecting the third target nucleic acid are used; and when n is 4, the composition for detecting the first target nucleic acid to the composition for detecting the fourth target nucleic acid are used.

In one embodiment, each of the n compositions for detecting the target nucleic acids provides a signal change at a corresponding detection temperature among the n detection temperatures, the signal change indicating the presence of a corresponding target nucleic acid.

For example, the composition for detecting an i^{th} target nucleic acid among the n target nucleic acids provides a signal change at an i^{th} detection temperature among the n detection temperatures and provides a constant signal at the other detection temperatures in the presence of the i^{th} target nucleic acid.

In one embodiment, the composition for detecting the i^{th} target nucleic acid, in the presence of the i^{th} target nucleic acid, provides a signal change upon amplification of the target nucleic acid at the i^{th} detection temperature (that is, change in an i^{th} signal) while providing no signal change at the other detection temperatures, even as the target nucleic acid is amplified (that is, the signal is constant).

The term “i^{th} signal” as used herein refers to a signal provided at an i^{th} detection temperature by the composition for detecting an i^{th} target nucleic acid, which is interchangeably used with “signal at an i^{th} detection temperature”.

In one embodiment, the composition for detecting the i^{th} target nucleic acid, when in the absence of the i^{th} target nucleic acid, provides no signal change, that is, provides a constant signal at the i^{th} detection temperature during an incubation reaction (e.g., a target nucleic acid amplification reaction). In one embodiment, when detecting n target nucleic acids, the i^{th} signal may mean a signal provided by the n compositions for detecting target nucleic acids including the composition for detecting the i^{th} target nucleic acid, at the i^{th} detection temperature.

In one embodiment, i represents an integer from 1 to n, and the i^{th} detection temperature is lower than the (i+1)^{th} detection temperature. In one embodiment, when i is n, there is no i+1 detection temperature (that is, n+1 detection temperature). For example, when n is 3, i represents an integer from 1 to 3, and there are a first detection temperature, a second detection temperature, and a third detection temperature, wherein the first detection temperature is lower than the second detection temperature, and the second detection temperature is lower than the third detection temperature.

In particular embodiment, when n is 2, the composition for detecting the first target nucleic acid provides a signal change at the first detection temperature and provides a constant signal at the other detection temperatures, that is, at the second detection temperature in the presence of the first target nucleic acid; and the composition for detecting the second target nucleic acid provides a signal change at the second detection temperature and provides a constant signal at the other detection temperatures, that is, at the first detection temperature in the presence of the second target nucleic acid.

In another specific embodiment, when n is 3, the composition for detecting the first target nucleic acid provides a signal change at the first detection temperature and provides a constant signal at the other detection temperatures, that is, at the second detection temperature and the third detection temperature in the presence of the first target nucleic acid; the composition for detecting the second target nucleic acid provides a signal change at the second detection temperature and provides a constant signal at the other detection temperatures, that is, at the first detection temperature and the third detection temperature in the presence of the second target nucleic acid; and the composition for detecting the third target nucleic acid provides a signal change at the third detection temperature and provides a constant signal at the other detection temperatures, that is, at the first detection temperature and the second detection temperature in the presence of the third target nucleic acid.

In one embodiment, the signal change includes “signal generation or extinguishment” and “signal increase or decrease” from a label.

In the present disclosure, a signal change means a significant “signal change” that is, a significant change in signal where the signal change indicates the presence of the target nucleic acid. For example, a significant signal change, that is, a signal change that indicates the presence of the target nucleic acid may refer to the generation or extinguishment of a signal that has a distinct intensity compared to the background signal intensity or the intensity of a signal in the absence of the target nucleic acid, or may refer to a substantial increase, or decrease of the intensity of a signal indicating the presence of the target nucleic acid, as the target nucleic acid and/or signal is amplified during an incubation reaction in step (a).

In one embodiment, the signal change may be generated depending on the presence of the target nucleic acid. For example, the signal change may be generated as a signal indicating the presence of the target nucleic acid is generated or extinguished.

In one embodiment, the signal change may be generated upon amplification of the target nucleic acid. That is, the signal change may be generated as the amount of the target nucleic acid increases upon amplification of the target nucleic acid. For example, upon amplification of the target nucleic acid, a signal indicating the presence of the target nucleic acid may increase or decrease, thereby inducing the signal change.

In one embodiment, the signal change may be generated upon the amplification of a signal dependent on the target nucleic acid. That is, upon amplification of the signal dependent on the target nucleic acid, the intensity of the signal changes, thereby changing the signal. For example, as the signal dependent on the presence of the target nucleic acid is amplified, the signal indicating the presence of the target nucleic acid may increase, or decrease, thereby inducing the signal change.

The term “constant signal” as used herein refers to no substantial change in signal during an incubation reaction (e.g., a target nucleic acid amplification reaction). That is, the term “constant signal” refers to all or any signal pattern other than significant signal changes brought about by the amplification of the target nucleic acid present. Specifically, the constant signal means no signal change. For example, if the signal during an amplification reaction does not exceed the background signal intensity, or the intensity of a signal in the absence of the target nucleic acid, it may be expressed as “the signal is constant”. In the present disclosure, the constant signal may be interchangeably used as the signal that does not change, or the signal that does not show change.

In the present disclosure, the designation of “signal change” and/or “constant signal” is based on signals detected at the same temperature during a nucleic acid amplification reaction using the same composition for detecting a target nucleic acid. For example, the designation of “signal change” and/or “constant signal” are used based on difference between signal values detected at the same temperature using n compositions for detecting target nucleic acids, and more specifically, “signal change” and/or “constant signal” are designated based on as “signal change” and/or “constant signal” based on (i) a difference between signal values, detected at the same temperature in a plurality of cycles, or (ii) a difference between “a reference signal value” described below and a signal value detected at the same temperature as the temperature for which the reference signal value is set. That is, the designation of “signal change” and/or “constant signal” is not based on a difference between signal values detected at different temperatures.

In the present disclosure, in order to provide signal changes for target nucleic acids, compositions for detecting the target nucleic acids are used. Each of the target nucleic acids is detected by a corresponding composition for detecting a target nucleic acid.

In one embodiment, the composition for detecting the i^{th} target nucleic acid includes a label that provides a signal depending on the presence of the i^{th} target nucleic acid.

In one embodiment, the label is linked to an oligonucleotide, or is incorporated into an oligonucleotide during the incubation (e.g., a nucleic acid amplification reaction). That is, the composition for detecting the target nucleic acid may initially include a label-linked oligonucleotide, or may provide a label-linked oligonucleotide as the label is incorporated into a newly generated oligonucleotide (e.g., an extended strand) during an incubation reaction.

In one embodiment, the composition for detecting the i^{th} target nucleic acid includes an incorporation label that is incorporated into an oligonucleotide during the incubation and provides a signal depending on the presence of the i^{th} target nucleic acid. In one embodiment, the composition for detecting the i^{th} target nucleic acid provides a label-linked oligonucleotide that serves to provide a signal depending on the presence of the i^{th} target nucleic acid.

In one embodiment, the composition for detecting the i^{th} target nucleic acid initially includes a label-linked oligonucleotide that serves to provide a signal depending on the presence of the i^{th} target nucleic acid. Alternatively, the composition for detecting the i^{th} target nucleic acid may include an oligonucleotide and a label that provides a signal depending on the presence of the target nucleic acid, and as the label is incorporated into the oligonucleotide during an incubation reaction (e.g., a nucleic acid amplification reaction), provide a label-linked oligonucleotide that serves to provide a signal depending on the presence of the target nucleic acid.

As used herein, the term “label-linked oligonucleotide” refers to an oligonucleotide involved in the generation of a signal being detected.

In one embodiment, the label-linked oligonucleotide may comprise an oligonucleotide that specifically hybridizes to a target nucleic acid (e.g., a probe or a primer); when the probe or primer hybridized to the target nucleic acid is cleaved to release a fragment, the label-linked oligonucleotide may comprise a capture oligonucleotide that specifically hybridizes to the fragment; when the fragment hybridized to the capture oligonucleotide is extended to form an extended strand, the label-linked oligonucleotide may comprise an oligonucleotide that specifically hybridizes to the extended strand, an oligonucleotide that produced by incorporating a label during the fragment extension, an oligonucleotide that specifically hybridizes to the capture oligonucleotide, and a combination thereof.

According to one embodiment, the label-linked oligonucleotide includes an oligonucleotide involved in actual signal generation. For example, hybridization or non-hybridization between the label-linked oligonucleotide and another oligonucleotide (e.g., an oligonucleotide comprising a nucleotide sequence complementary to the label-linked oligonucleotide or the target nucleic acid) determines signal generation.

In one embodiment, the label-linked oligonucleotide may be a ‘probe’ known in the art. The term “probe” as used herein refers to a single-stranded nucleic acid molecule comprising one or more portions substantially complementary to a target nucleic acid sequence. According to an embodiment of the present invention, the 3′-end of the probe is “blocked” to prohibit its extension. The blocking may be achieved in accordance with conventional methods. For instance, the blocking may be performed by adding to the 3′-hydroxyl group of the last nucleotide a chemical moiety such as biotin, labels, phosphate groups, alkyl groups, non-nucleotide linkers, phosphorothioate or alkane-diol residues. Alternatively, the blocking may be carried out by removing the 3′-hydroxyl group of the last nucleotide or using a nucleotide with no 3′-hydroxyl group such as dideoxynucleotide.

According to an embodiment, the label-linked oligonucleotide may be composed of at least one oligonucleotide. According to an embodiment of the present invention, when the label-linked oligonucleotide is composed of a plurality of oligonucleotides, the label-linked oligonucleotide may be labeled in various fashions. For example, all or portion of the plurality of oligonucleotides may have at least one label.

In one embodiment, the interactive labels may be interactive dual labels including one reporter molecule and one quencher molecule.

In one embodiment, the interactive labels may be interactive labels including at least one reporter molecule and at least one quencher molecule. In particular, the interactive labels may be interactive dual labels including one reporter molecule and one quencher molecule. Or, the interactive labels may be interactive labels including one reporter molecule and two quencher molecules.

In one embodiment, when the label is a single label, the single label may be linked to one oligonucleotide.

In one embodiment, when the label is interactive labels, the interactive labels may be interactive labels including at least one reporter molecule and at least one quencher molecule, wherein the interactive labels may be all linked to one oligonucleotide or may be linked to each of a plurality of oligonucleotides.

For example, the single label includes a fluorescent label, a luminescent label, a chemiluminescent label, an electrochemical label and a metal label. In one embodiment, the single label provides different signals (for example, different signal strengths) depending on its presence on a double strand or a single strand. In one embodiment, the single label is a fluorescent label. Preferred types and binding sites of single fluorescent labels used in the present disclosure are disclosed in U.S. Pat. Nos. 7,537,886 and 7,348,141, the teachings of which are incorporated herein by reference in their entirety. For example, the single fluorescent label includes JOE, FAM, TAMRA, ROX and fluorescein-based label. The single label may be linked to an oligonucleotide by various methods. For example, the label may be linked to a probe via a spacer containing carbon atoms (e.g., a 3-carbon spacer, a 6-carbon spacer, or a 12-carbon spacer).

As a representative of the interactive label system, the FRET (fluorescence resonance energy transfer) label system includes a fluorescent reporter molecule (donor molecule) and a quencher molecule (acceptor molecule). In FRET, the energy donor is fluorescent, but the energy acceptor may be fluorescent or non-fluorescent. In another form of interactive label systems, the energy donor is non-fluorescent, e.g., a chromophore, and the energy acceptor is fluorescent. In yet another form of interactive label systems, the energy donor is luminescent, e.g., bioluminescent, chemiluminescent, electrochemiluminescent, and the acceptor is fluorescent. The interactive label system includes a label pair based on “contact-mediated quenching” (Salvatore et al., Nucleic Acids Research, 2002 (30) no. 21 e122 and Johansson et al., J. AM. CHEM. SOC 2002 (124) pp 6950-6956). The interactive label system includes any or all label systems that induce signal changes through interactions between at least two molecules (e.g., dyes).

The reporter molecule and the quencher molecule useful in the present invention may include any molecules known in the art. Examples of such molecules are as follows: Cy2™ (506), YO-PRO™-1 (509), YOYO™-1 (509), Calcein (517), FITC (518), FluorX™ (519), Alexa™ (520), Rhodamine 110 (520), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-1 (533), TOTO1 (533), JOE (548), BODIPY530/550 (550), Dil (565), BODIPY TMR (568), BODIPY558/568 (568), BODIPY564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red (615), Nile Red (628), YO-PRO™-3 (631), YOYO™-3 (631), Rphycocyanin (642), C-Phycocyanin (648), TO-PRO™-3 (660), TOTO3 (660), DiD DilC(5) (665), Cy5™ (670), Thiadicarbocyanine (671), Cy5.5 (694), HEX (556), TET (536), Biosearch Blue (447), CAL Fluor Gold 540 (544), CAL Fluor Orange 560 (559), CAL Fluor Red 590 (591), CAL Fluor Red 610 (610), CAL Fluor Red 635 (637), FAM (520), Fluorescein (520), Fluorescein-C3 (520), Pulsar 650 (566), Quasar 570 (667), Quasar 670 (705) and Quasar 705 (610). The numeral in parenthesis is a maximum emission wavelength in nanometer. Preferably, the reporter molecule and the quencher molecule include JOE, FAM, TAMRA, ROX and fluorescein-based label.

Suitable pairs of reporter-quencher are disclosed in a variety of publications as follows: Pesce et al., editors, Fluorescence Spectroscopy (Marcel Dekker, New York, 1971); White et al., Fluorescence Analysis: A Practical Approach (Marcel Dekker, New York, 1970); Berlman, Handbook of Fluorescence Spectra of Aromatic Molecules, 2nd Edition (Academic Press, New York, 1971); Griffiths, Color AND Constitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluorescence and Phosphorescence (Interscience Publishers, New York, 1949); Haugland, R. P., Handbook of Fluorescent Probes and Research Chemicals, 6th Edition (Molecular Probes, Eugene, Oreg., 1996); U.S. Pat. Nos. 3,996,345 and 4,351,760.

In one embodiment, an incorporation label may be used in the process of incorporating a label during a primer extension to generate a signal (e.g., Plexor technology, Sherrill C B et al., Journal of the American Chemical Society, 126:4550-45569 (2004)). In addition, the incorporation label may be used in a signal generation by a duplex formed in a manner dependent on cleavage of a mediation oligonucleotide hybridized to a target nucleic acid sequence.

In one embodiment, the incorporation label may be generally linked to a nucleotide. In addition, a nucleotide having a non-natural base may be used.

As used herein, the term “non-natural base” refers to derivatives of natural bases such as adenine (A), guanine (G), thymine (T), cytosine (C) and uracil (U), which are capable of forming hydrogen-bonding base pairs. The term “non-natural base” as used herein includes bases having base pairing patterns different from natural bases as mother compounds, as described, for example, in U.S. Pat. Nos. 5,432,272, 5,965,364, 6,001,983, and 6,037,120. The base pairing between non-natural bases includes two or three hydrogen bonds as with natural bases. The base pairing between non-natural bases is also formed in a specific manner. Specific examples of non-natural bases include the following bases in base pair combinations: iso-C/iso-G, iso-dC/iso-dG, Z/P, V/J, K/X, H/J, Pa/Ds, Pa/Q, Pn/Ds, Pn/Dss, Px/Ds, NaM/5SICS, 5FM/5SICS, and M/N (see U.S. Pat. Nos. 5,432,272; 5,965,364; 6,001,983; 6,037,120; 6,140,496; 6,627,456; 6,617,106; and 7,422,850; and Filip Wojciechowski et al., Chem. Soc. Rev., 2011, 40, 5669-5679).

The conventional method for detecting multiple target nucleic acids require the use of different types of fluorescent label for different target nucleic acids, or even when using a single type of fluorescent label, these methods have a disadvantage in that they require an additional analysis, such as melting curve analysis. Unlike such methods, by using a composition for detecting a target nucleic acid which provides a duplex, the method according to the present disclosure can detect multiple target nucleic acids in a real-time manner using a single type of label (e.g., a single fluorescent label) without an additional analysis such as melting analysis.

In one embodiment, each of the n compositions for detecting target nucleic acids provides one or more duplexes.

The term “duplex” is used herein to encompass both a duplex in associated form and a duplex in dissociated form. That is, said term may refer to two single-stranded nucleic acid molecules, having a sequence partially or totally complementary to each other, such that under a hybridization condition, they can be hybridized to each other to have a duplex structure. For example, depending on the detection temperature, all or a portion of the duplexes may be in associated form or in dissociated form

The term “association or dissociation” has the same meaning as the term “hybridization or denaturation.

The expression “a composition for detecting a target nucleic acid provides a duplex” as used herein may mean providing a duplex in associated form and/or a duplex in dissociated form. Likewise, the expression “a composition for detecting a target nucleic acid generates a duplex during incubation” as used herein may mean generating a duplex in associated form and/or a duplex in dissociated form during an incubation reaction.

In one embodiment, at least one of the duplexes provided by the composition for detecting a target nucleic acid is a duplex providing a signal. In particular, the duplex is a duplex providing a signal change. That is, the composition for detecting an i^{th} target nucleic acid provides a duplex providing a signal, and particularly, the composition for detecting the i^{th} target nucleic acid provides a duplex providing a signal change depending on the presence of the i^{th} target nucleic acid.

The term “duplex providing a signal” as used herein refers to a duplex capable of providing a signal that can be distinguished depending on whether the duplex is in associated form or dissociated form. For example, this means that the duplex in associated form generates (or extinguishes) a signal, and the duplex in dissociated form extinguishes (or generates) a signal.

In one embodiment, the duplex providing the signal may include at least one label. In particular, at least one label is linked to at least one strand of the two single-strands constituting the duplex. For example, the duplex providing the signal includes a single label, and in this case, the single label is linked to any one strand of the two single-strands constituting the duplex. As another example, the duplex providing a signal includes interactive labels, and in this case, the interactive labels are all linked to one of the two strands constituting the duplex providing the signal, or one of the interactive labels is linked to any of the two single strands and the other of the interactive label is linked to the other of the two single strands.

As used herein, the term “duplex providing a signal change” refers to a duplex providing a signal change indicative of the presence of a target nucleic acid as the amount of the duplex providing the signal change changes depending on the presence of the target nucleic acid. In particular, in the PTOCE-based method described below, an extended duplex including a label generated depending on the presence of a target nucleic acid, is an example of a duplex providing a signal change, described in the present application.

In one embodiment, the duplex providing the signal change includes a label. In particular, at least one label is linked to at least one strand of the two single-strands constituting the duplex. For example, the duplex providing the signal change includes a single label, and in this case, the single label is linked to any one strand of the two single-strands constituting the duplex. As another example, the duplex providing the signal change includes interactive labels, and in this case, the interactive labels are all linked to one of the two strands constituting the duplex providing the signal change, or one of the interactive labels is linked to any of the two single strands and the other of the interactive label is linked to the other of the two single strands.

In one embodiment, the composition for detecting the i^{th} target nucleic acid provides a duplex providing a signal change.

In one embodiment, the composition for detecting the i^{th} target nucleic acid provides a signal from the label when the duplex providing the signal change is present in associated form. That is, the composition for detecting the i^{th} target nucleic acid provides a signal depending on association of the two single-stranded nucleic acid molecules constituting the duplex.

In an alternative embodiment, the composition for detecting the i^{th} target nucleic acid provides a signal from the label when the duplex providing the signal change is present in dissociated form. That is, the composition for detecting the i^{th} target nucleic acid provides a signal depending on dissociation of the two single-stranded nucleic acid molecules constituting the duplex.

In one embodiment, the association or dissociation of the duplex may be caused by temperature.

In one embodiment, the duplex providing the signal change may be a duplex that has initially (originally) been included in the composition for detecting a target nucleic acid.

In one embodiment, when the duplex providing the signal change has been included in the composition for detecting the target nucleic acid, the duplex may be generated by hybridization between a label-linked oligonucleotide and an oligonucleotide hybridizable with the label-linked oligonucleotide. For example, the Yin-Yang probe described below is an example of a duplex providing a signal change depending on the presence of a target nucleic acid, and has initially been included in the composition for detecting the target nucleic acid.

In one embodiment, when the duplex providing the signal change has initially been included in the composition for detecting the target nucleic acid, the amount of the duplex providing the signal change varies, in particular, decreases, in a manner dependent on the presence of the target nucleic acid, thereby providing the signal change. For example, for a Yin-Yang probe initially included in the composition for detecting a target nucleic acid, as the target nucleic acid is amplified, one of the two single strands constituting the Yin-Yang probe pairs with the amplified target nucleic acid to generate a new duplex, and the amount of the Yin-Yang probe decreases, thus providing a signal change depending on the presence of the target nucleic acid.

In one embodiment, the duplex providing the signal change may be a duplex newly provided by the composition for detecting a target nucleic acid during an incubation reaction.

In one embodiment, the duplex providing the signal change, which is generated during the incubation reaction, may be provided by hybridization between a label-linked oligonucleotide and the target nucleic acid.

Signals by formation of a duplex between the label-linked oligonucleotide and the target nucleic acid may be generated by various methods including Scorpion method (Whitcombe et al., Nature Biotechnology 17:804-807 (1999)), Sunrise (or Amplifluor) method (Nazarenko et al., Nucleic Acids Research, 25(12):2516-2521 (1997), and U.S. Pat. No. 6,117,635), LUX method (U.S. Pat. No. 7,537,886), Plexor method (Sherrill C B, et al., Journal of the American Chemical Society, 126:4550-4556 (2004)), Molecular beacon method (Tyagi et al., Nature Biotechnology v. 14 Mar. 1996), Hybeacon method (French D J et al., Mol. Cell Probes, 15(6):363-374 (2001)), adjacent hybridization probe method (Bernard P. S. et al., Anal. Biochem., 273:221 (1999)), and LNA method (U.S. Pat. No. 6,977,295).

In one embodiment, the duplex providing the signal change, which is generated during the incubation reaction, may be a duplex generated by a cleavage reaction dependent on the presence of the target nucleic acid.

For the above reaction, a 5′-nuclease and a 3′-nuclease, in particular, a DNA polymerase having 5′ nuclease activity, a DNA polymerase having 3′-nuclease activity, or FEN nuclease may be used.

In one embodiment, the signal change is generated by a duplex generated in a manner dependent on the cleavage of a mediation oligonucleotide specifically hybridized to the target nucleic acid.

As used herein, the term “mediation oligonucleotide” refers to an oligonucleotide mediating the generation of a duplex, not including a target nucleic acid.

In one embodiment, the cleavage of the mediation oligonucleotide alone does not generate a signal, but after hybridization and cleavage of the mediation oligonucleotide, a fragment (a cleavage product) produced by the cleavage is involved in the continuous reaction for signal generation.

In one embodiment, the hybridization or cleavage of the mediation oligonucleotide alone does not generate a signal.

In one embodiment, the mediation oligonucleotide includes an oligonucleotide that hybridizes to a target nucleic acid sequence and cleaved to release a fragment, thereby mediating the generation of a duplex.

In one embodiment, the fragment mediates the generation of a duplex by extension of the fragment on a capture oligonucleotide.

According to an embodiment, the mediation oligonucleotide comprises (i) a targeting portion comprising a hybridizing nucleotide sequence complementary to a target nucleic acid sequence, and (ii) a tagging portion comprising a nucleotide sequence non-complementary to the target nucleic acid sequence.

In one embodiment, the composition for detecting a target nucleic acid may include a tagging oligonucleotide that hybridizes to the target nucleic acid, and the cleavage reaction dependent on the presence of the target nucleic acid may involve cleavage of the tagging oligonucleotide. The tagging oligonucleotide corresponds to an example of the mediation oligonucleotide described above.

According to an embodiment, cleavage of the mediation oligonucleotide releases a fragment, and the fragment is specifically hybridized to a capture oligonucleotide and extended on the capture oligonucleotide. When the capture oligonucleotide comprises a label, the capture oligonucleotide corresponds to an example of the label-linked oligonucleotide described herein.

According to an embodiment, the mediation oligonucleotide hybridized to a target nucleic acid sequence is cleaved and releases a fragment, the fragment is specifically hybridized to a capture oligonucleotide, and the fragment is extended to form an extended strand, and this induces the formation of an extended duplex between the extended strand and the capture oligonucleotide, thereby providing a signal indicative of the presence of the target nucleic acid sequence.

In one embodiment, the signal indicative of the presence of the target nucleic acid may be provided by (i) at least one label linked to the fragment and/or the capture oligonucleotide, (ii) a label incorporated into the extended duplex during the extension reaction, and (iii) the label incorporated into the extended duplex during the extension reaction and the label linked to the fragment and/or the capture oligonucleotide. According to an embodiment, when a third oligonucleotide including a hybridizing nucleotide sequence complementary to the extended strand is used, the hybridization of the extended strand and the third oligonucleotide forms another type of duplex, thereby providing a signal indicating the presence of the target nucleic acid (e.g., PCE-SH). In this case, another type of duplex is a duplex providing the signal change.

In an embodiment, a duplex formed by a cleavage reaction dependent on the presence of the target nucleic acid is not a duplex providing a signal change, but may be a duplex changing the content of a duplex providing a signal change. In this case, the duplex providing the signal change has initially been included in a composition for detecting the target nucleic acid. According to an embodiment, when an additional oligonucleotide including a hybridizing nucleotide sequence complementary to the capture oligonucleotide is used, the duplex between the additional oligonucleotide and the capture oligonucleotide is a duplex providing a signal change, and the amount of the duplex providing the signal change is changed (e.g., decreased) by generating a duplex between the extended strand and the capture oligonucleotide, thereby providing the signal change indicative of the presence of the target nucleic acid. For example, in the PCE-NH method, a duplex between the additional oligonucleotide and the capture oligonucleotide is a duplex providing a signal change that has been initially included in a composition for detecting the target nucleic acid, and the capture oligonucleotide constituting the duplex providing the signal change are used to generate a new duplex (e.g., an extended duplex) by a cleavage reaction dependent on the presence of the target nucleic acid, thereby decreasing the amount of the duplex providing the signal change to provide the signal change.

According to an embodiment, the fragment, the extended strand, the capture oligonucleotide, the additional oligonucleotide, or a combination thereof, may act as the label-linked oligonucleotide.

Signals by a duplex formed in a manner dependent on cleavage of the mediation oligonucleotide may be generated by various methods, including PTOCE (PTO cleavage and extension) method (WO 2012/096523), PCE-SH (PTO Cleavage and Extension-Dependent Signaling Oligonucleotide Hybridization) method (WO 2013/115442), and PCE-NH (PTO Cleavage and Extension-Dependent Non-Hybridization) method (WO 2014/104818).

Regarding the terms disclosed in the above references, the corresponding examples of the oligonucleotides are as follows: the mediation oligonucleotide corresponds to a PTO (Probing and Tagging Oligonucleotide), the capture oligonucleotide corresponds to a CTO (Capturing and Templating Oligonucleotide), and the additional oligonucleotide corresponds to an SO (Signaling Oligonucleotide) or an HO (Hybridization Oligonucleotide). The SO, HO, CTO, extended strand or a combination thereof may play the role of a label-linked oligonucleotide.

In one embodiment, the signals by the duplex formed in a manner dependent on cleavage of the mediation oligonucleotide includes a signal that is provided as the amount of other duplexes decrease by the duplex formed in a manner dependent on the cleavage of the mediation oligonucleotide (e.g., PCE-NH).

In one embodiment, when the signals by the duplex generated in a manner dependent on cleavage of the mediation oligonucleotide are generated by a PTOCE method, an upstream oligonucleotide, a probing and tagging oligonucleotide (PTO) comprising a hybridizing nucleotide sequence complementary to the target nucleic acid, a capturing and templating oligonucleotide (CTO), an appropriate label, and a template-dependent DNA polymerase having a 5′-nuclease activity may be included in a reaction, and the composition for detecting the target nucleic acid may include these components.

The PTO comprises (i) a 3′-targeting portion comprising a hybridizing nucleotide sequence complementary to the target nucleic acid sequence and (ii) a 5′-tagging portion comprising a nucleotide sequence non-complementary to the target nucleic acid sequence. The CTO comprises in a 3′ to 5′ direction (i) a capturing portion comprising a nucleotide sequence complementary to the 5′-tagging portion or a part of the 5′-tagging portion of the PTO, and (ii) a templating portion comprising a nucleotide sequence non-complementary to the 5′-tagging portion and the 3′-targeting portion of the PTO.

A particular example of signal generation by the PTOCE method comprises the following steps:

(a) hybridizing the target nucleic acid with an upstream oligonucleotide and a PTO; (b) contacting the product of step (a) with an enzyme having a 5′ nuclease activity under conditions for cleavage of the PTO; wherein the upstream oligonucleotide or its extended strand induces cleavage of the PTO by the enzyme having the 5′ nuclease activity such that the cleavage releases a fragment comprising the 5′-tagging portion or a part of the 5′-tagging portion of the PTO; (c) hybridizing the fragment released from the PTO with a CTO; wherein the fragment released from the PTO is hybridized to a capturing portion of the CTO; (d) performing an extension reaction using the resultant of the step (c) and a template-dependent nucleic acid polymerase; wherein the fragment hybridized to the capturing portion of the CTO is extended to form an extended duplex; wherein the extended duplex has a Tm value adjustable by (i) a sequence and/or length of the fragment, (ii) a sequence and/or length of the CTO or (iii) the sequence and/or length of the fragment and the sequence and/or length of the CTO; and the extended duplex provides a target signal by at least one label linked to the fragment and/or the CTO; and (e) detecting the extended duplex by measuring the target signal at a predetermined temperature at which the extended duplex retains its double-stranded form, wherein the presence of the extended duplex indicates the presence of the target nucleic acid. In this case, the method further comprises repeating all or a part of the steps (a)-(e) with including denaturation between repeating cycles.

In the phrase “denaturation between repeating cycles,” the term “denaturation” refers to separating a double-stranded nucleic acid molecule into single-stranded nucleic acid molecules.

In step (a) in the PTOCE method, a primer set for target nucleic acid amplification may be used instead of the upstream oligonucleotide. In this case, the method further comprises repeating all or a part of the steps (a)-(e) with denaturation between repeating cycles.

The PTOCE method may be classified as the process in which a PTO fragment hybridized to a CTO is extended to form an extended strand, and the extended strand is detected. The PTOCE method is characterized in that the formation of the extended strand is detected using a duplex between the extended strand and the CTO.

There are other approaches for the detection of the formation of the extended strand. For example, the formation of the extended strand may be detected using an oligonucleotide specifically hybridized to the extended strand (e.g., a PCE-SH assay). In such a method, the signal may be provided from (i) a label linked to the oligonucleotide specifically hybridized to the extended strand, or (ii) a label linked to the oligonucleotide specifically hybridized to the extended strand, and a label linked to the PTO fragment.

Alternatively, the detection of the formation of the extended strand is carried out by other methods (e.g., a PCE-NH assay) for detecting change in the amount of a duplex between the CTO and an oligonucleotide specifically hybridizable to the CTO. Such a change is considered to be indicative of the presence of a target nucleic acid. The signal may be provided from (i) a label linked to an oligonucleotide hybridizable to the CTO, (ii) a label linked to the CTO, or (iii) a label linked to the oligonucleotide hybridizable to the CTO and a label linked to the CTO.

According to an embodiment, the oligonucleotide specifically hybridizable to the CTO has an overlapping sequence with the PTO fragment.

According to an embodiment, the label-linked oligonucleotide includes an oligonucleotide specifically hybridizable to the extended strand (e.g., a PCE-SH assay) and an oligonucleotide specifically hybridizable to the CTO (e.g., a PCE-NH assay).

The PTOCE-based method is generally accompanied by the formation of an extended strand that is dependent on the presence of a target nucleic acid. The term “PTOCE-based method” is used herein to encompass various methods for providing signals, comprising the formation of an extended strand through cleavage and extension of a PTO.

One example of signal generation by a PTOCE-based method comprises the following steps: (a) hybridizing a target nucleic acid with an upstream oligonucleotide and a PTO; (b) contacting the product of step (a) with an enzyme having a 5′ nuclease activity under conditions for cleavage of the PTO, wherein the upstream oligonucleotide or its extended strand induces cleavage of the PTO by the enzyme having the 5′ nuclease activity and the cleavage releases a fragment comprising the 5′-tagging portion or a part of the 5′-tagging portion of the PTO; (c) hybridizing the fragment released from the PTO with a CTO, wherein the fragment released from the PTO is hybridized to a capturing portion of the CTO; (d) performing an extension reaction using the product of step (c) and a template-dependent nucleic acid polymerase, wherein the fragment hybridized to the capturing portion of the CTO is extended, forming an extended duplex; and (e) detecting formation of the extended duplex by measuring a signal generated dependent on the presence of the extended strand. In step (a), a primer set for target nucleic acid amplification may be used instead of the upstream oligonucleotide. In this case, the method further comprises repeating all or a part of the steps (a)-(e) with denaturation between repeating cycles.

Another example of signal generation mechanisms dependent on the formation of a duplex by cleavage of a mediation oligonucleotide is a C-tag technique (Korean Pat. No. 1961642). This method utilizes a primer (hereinafter referred to as a C-Tag primer) having a structure that sequentially includes a random nucleic acid sequence non-complementary to a target nucleic acid, a restriction enzyme recognition sequence and a nucleic acid sequence complementary to a target nucleic acid sequence as a mediation oligonucleotide. The amplification product, formed as the C-Tag primer is hybridized to the target nucleic acid and extended, includes the restriction enzyme recognition sequence and a sequence complementary to the random nucleic acid sequence. Further, as a restriction enzyme cleaves the amplification product, thereby releasing a tag fragment complementary to the random nucleic acid sequence, and the tag fragment specifically hybridizes to a capture oligonucleotide to form a duplex, thereby providing a signal indicative of the presence of the target nucleic acid sequence.

In one embodiment, the signal change may be generated by the dissociation of a duplex formed after the cleavage of a labeled oligonucleotide hybridized to the target nucleic acid, which takes place in a manner dependent on the presence of a target nucleic acid.

Another example of signal generation mechanisms dependent on the formation of a duplex by cleavage of a mediation oligonucleotide includes a dual quenching assay combined with melting analysis (WO 2016/101959).

Regarding the terms disclosed in the above publication, the corresponding examples of the oligonucleotides are as follows: the mediation oligonucleotide corresponds to a Probing and Tagging Oligonucleotide (PTO), and the capture oligonucleotide corresponds to a Capturing and Quenching Oligonucleotide (CQO).

In one embodiment, a particular embodiment of signal generation by the dual quenching assay comprises the following steps:
(a) hybridizing a target nucleic acid sequence with a PTO; the PTO comprising (i) a targeting portion comprising a nucleotide sequence substantially complementary to the target nucleic acid sequence, (ii) a Melting Temperature Deciding Region (MTDR), comprising a nucleotide sequence non-complementary to the target nucleic acid sequence, and (iii) at least one set of interactive labels comprising at least one fluorophore and at least one quencher;
(b) hybridizing the PTO with a CQO;
wherein the CQO comprises (i) a capturing portion comprising a nucleotide sequence reverse complementary to the MTDR of the PTO and (ii) at least one quenching molecule, wherein the MTDR is configured to hybridize with the capturing portion of the CQO to form a Tag Duplex;
(c) contacting the Tag Duplex with an enzyme having nuclease activity; wherein the enzyme having nuclease activity induces cleavage of the Tag Duplex when the Tag Duplex is hybridized with the target nucleic acid sequence, thereby releasing an activated Tag Duplex fragment comprising a PTO fragment comprising the MTDR hybridized to the capturing portion of the CQO and the at least one fluorophore;
(d) melting and/or hybridizing said activated Tag Duplex fragment to obtain a signal from the at least one fluorophore; and
(e) detecting the activated Tag Duplex fragment by measuring the signal from the at least one fluorophore, wherein the signal is indicative of the presence of the target nucleic acid sequence. In one embodiment, the order of steps (a) to (c) may be changed. For example, the steps may be performed in the following order: hybridizing a PTO with a CQO (step (b))—hybridizing a target nucleic acid sequence with a Tag Duplex (step (a))—cleaving the Tag Duplex hybridized to the target nucleic acid sequence by an enzyme having nuclease activity (step (c)); or hybridizing a PTO with a target nucleic acid sequence (step (a))—releasing an activated PTO fragment including at least one fluorophore and a MTDR by an enzyme having nuclease activity (step (c))—hybridizing an activated PTO with a CQO (step (b)).

In one embodiment, the duplex providing the signal change may be a single-typed duplex or plural-typed duplexes. Specifically, when the duplex providing the signal change is a single-typed duplex, the number of the duplexes may be 1, and when the duplex providing the signal change is plural-typed duplexes, the number of the duplexes may be 2, 3, 4, 5, 6, 7, 8, 9, 10, 15, or 20, more specifically, may be 2, 3, or 4, and more specifically, may be 2 or 3.

In one embodiment, the single-typed duplex, or any duplex among the plural-typed duplexes includes a label.

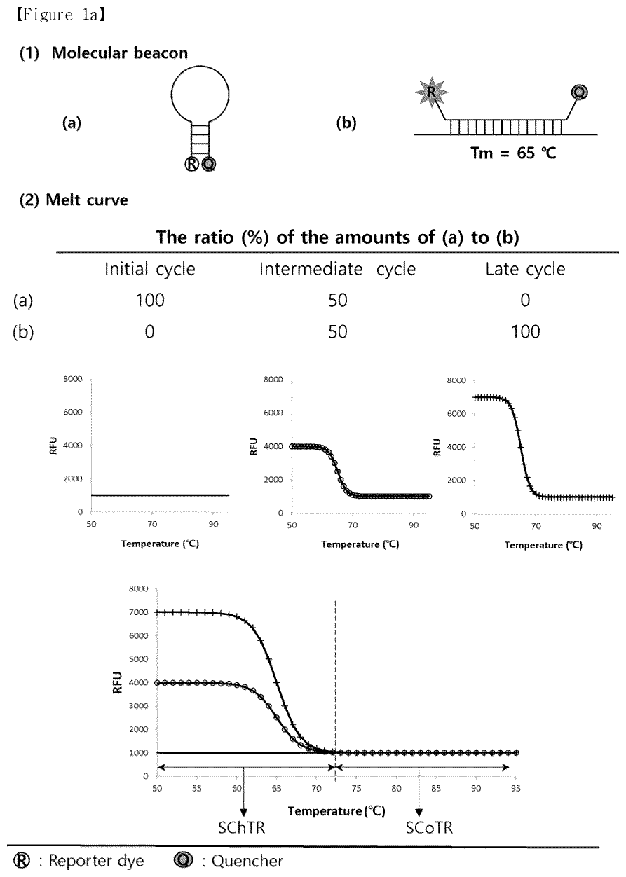

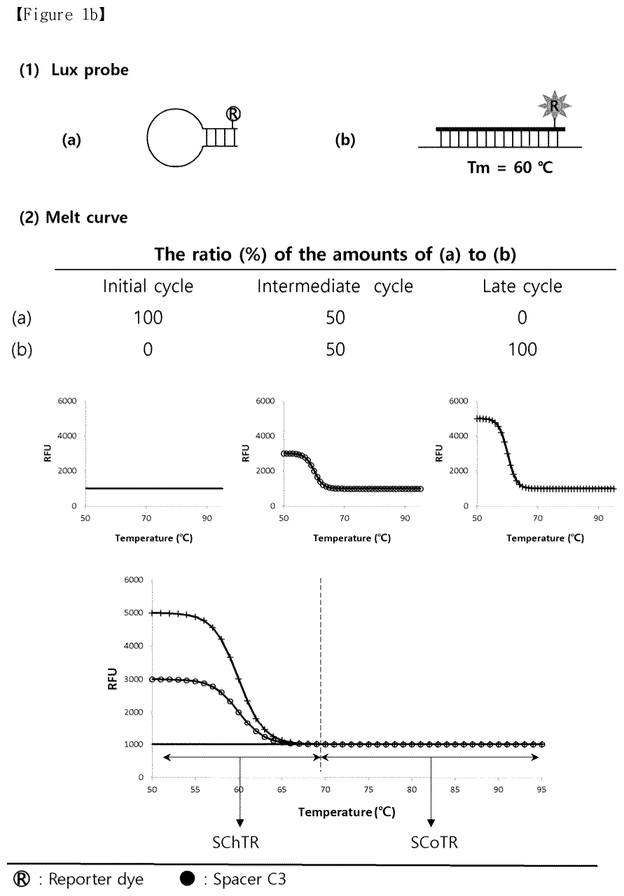

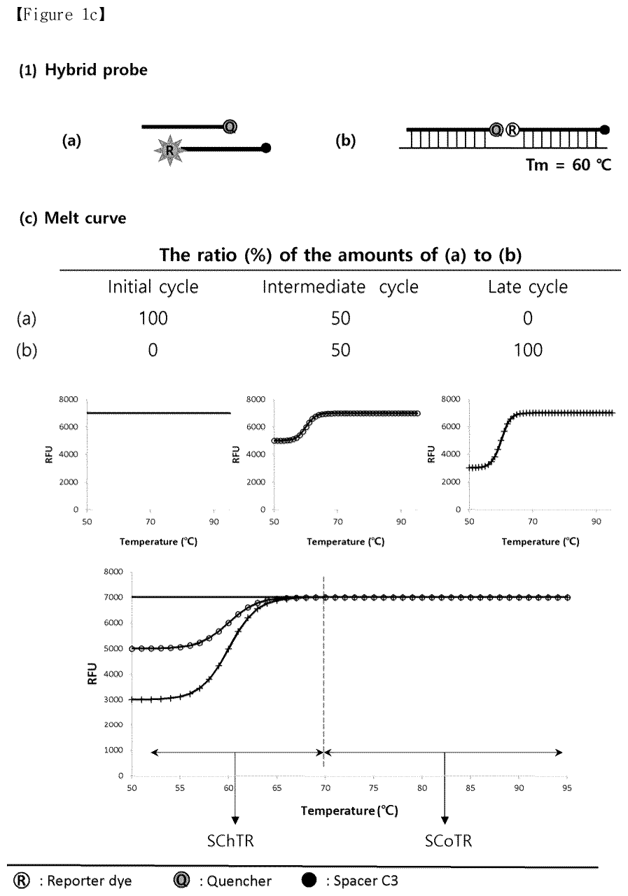

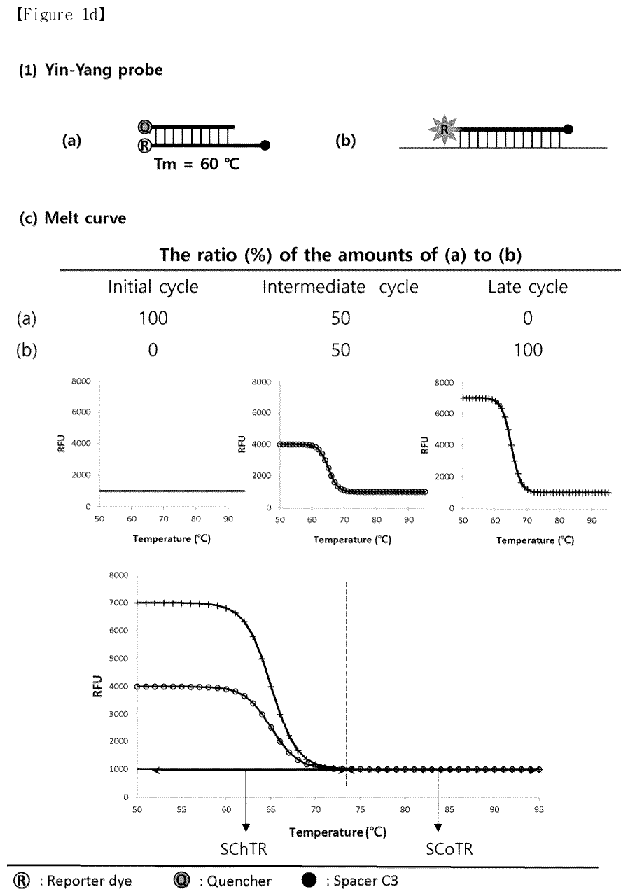

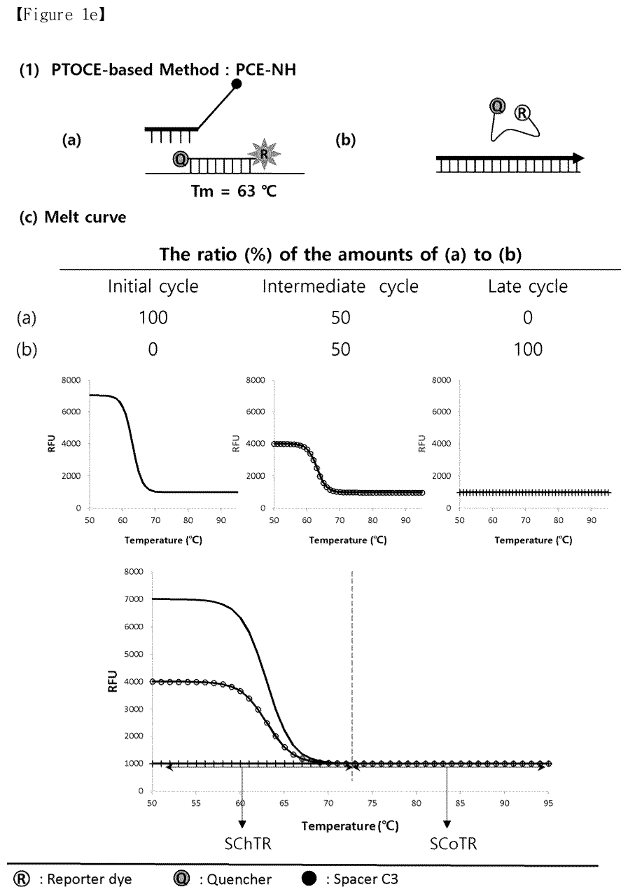

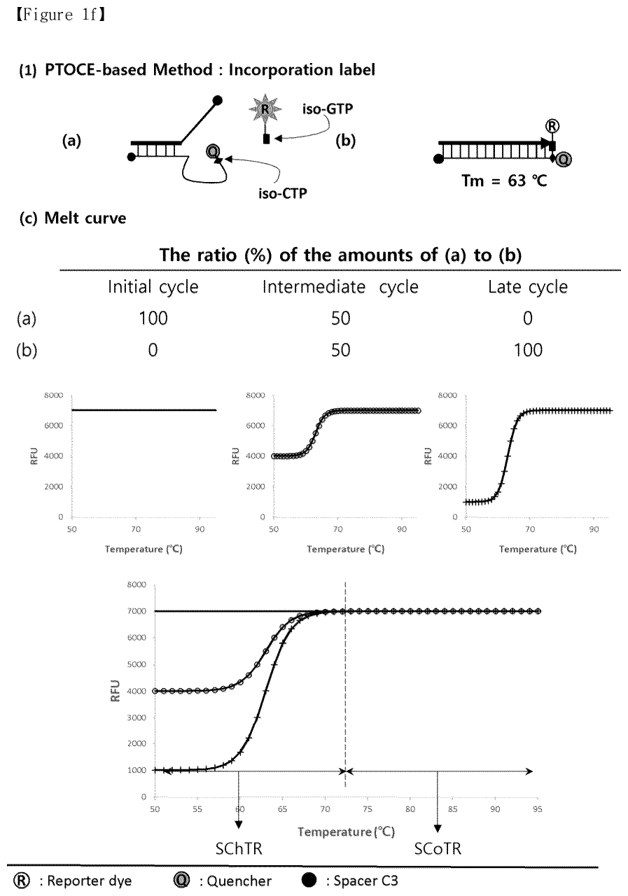

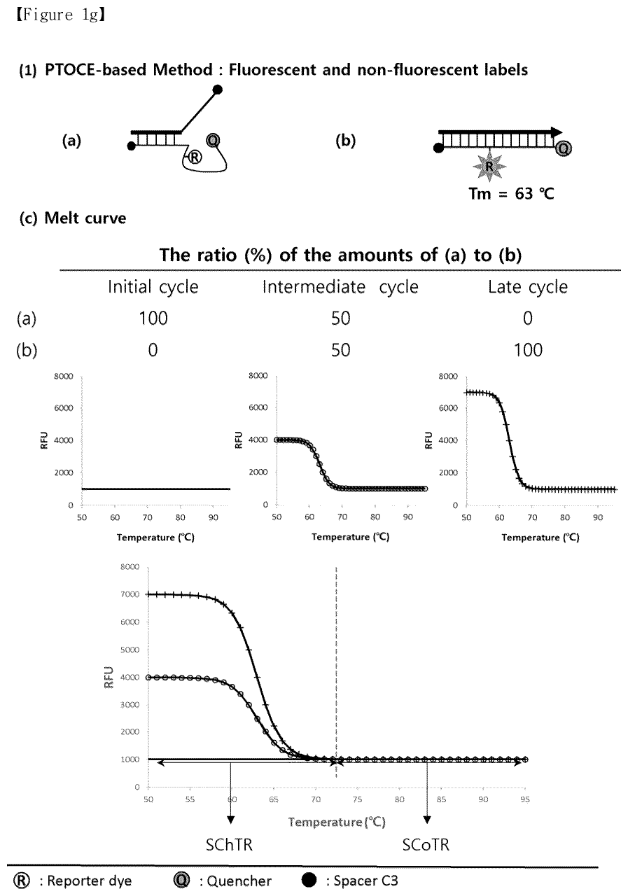

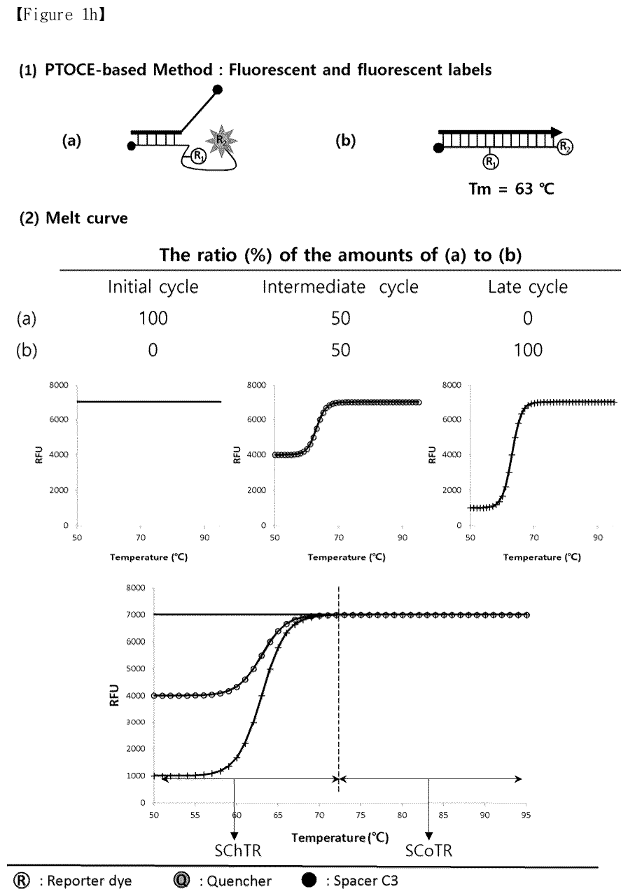

In one embodiment, a duplex in which two different single-strands hybridize to different sites on the same single strand may be present. For example, as shown in FIG. 1c, an adjacent hybridization probe method utilizing two probes is a method that provides a signal depending on whether the two probes are hybridized at different sites adjacent to each other on a single strand (for example, a target nucleic acid). In this case, since the method according to the present disclosure provides the same signal when at least one of the two probes is dissociated, the duplex providing a signal is one, and therefore, the method according to the present disclosure may be classified as a method providing a single-typed duplex.

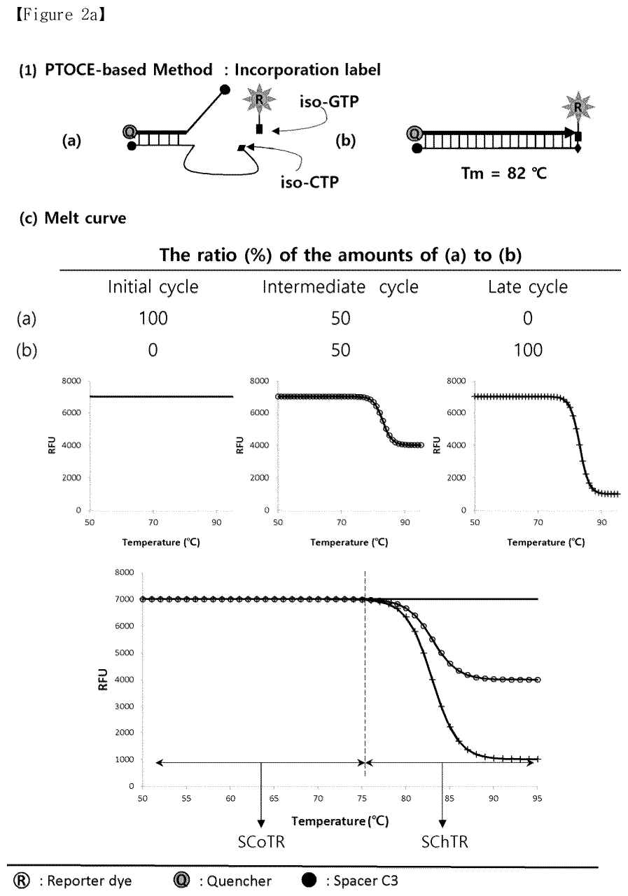

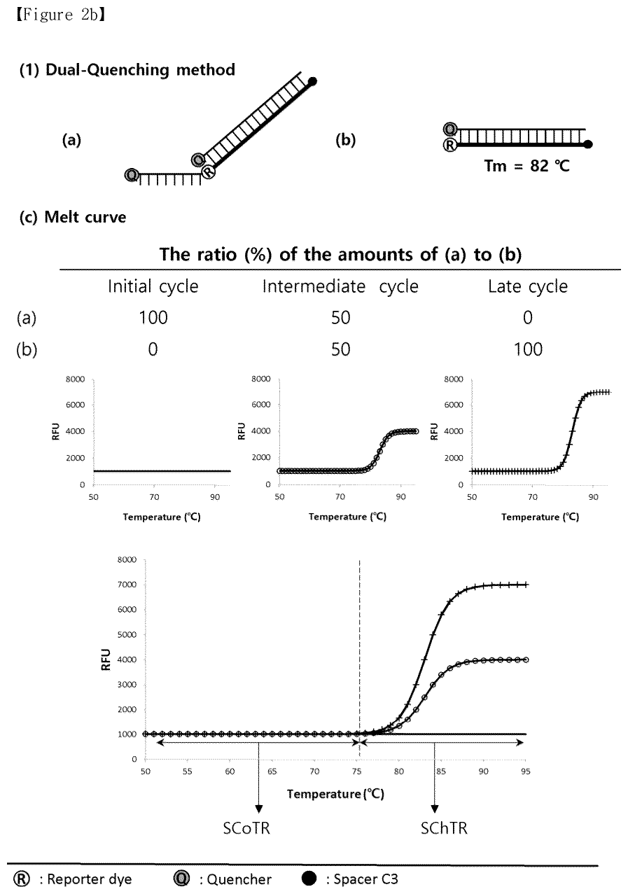

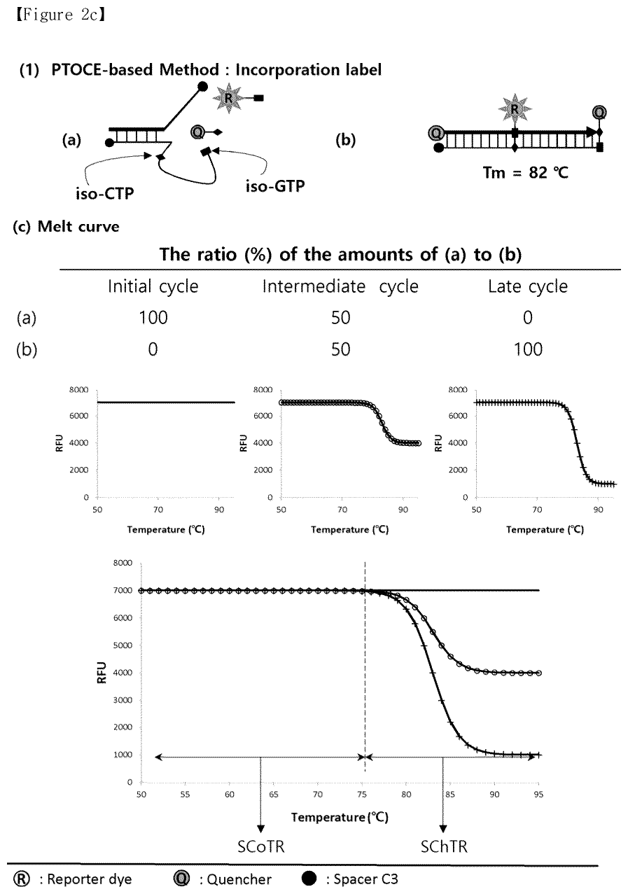

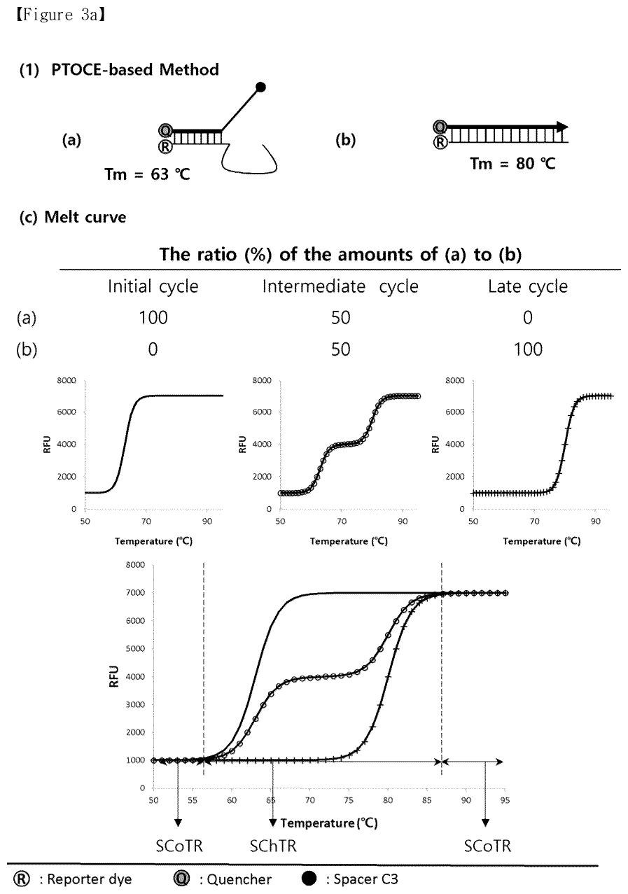

In one embodiment, when the duplex providing the signal change is a single-typed duplex, the amount of the single-typed duplex changes depending on the presence of a target nucleic acid, thereby changing the signal. For example, with reference to the drawings, the molecular beacon method in FIG. 1a, the LUX probe method in FIG. 1b, the hybrid probe method in FIG. 1c, and the Yin-Yang probe method in FIG. 1d, the PTOCE-based method in FIG. 1e, FIG. 1f, FIG. 1g, FIG. 1h, FIG. 2a, and FIG. 2c, and the dual-quenching method in FIG. 2b, are examples of providing a single-typed duplex. In particular, in case of FIG. 1a, FIG. 1b, FIG. 1c, FIG. 1f, FIG. 1g, FIG. 1h, FIG. 2a, FIG. 2b, and FIG. 2c, the single-typed duplex is generated during the incubation, and the amount of the single-typed duplex increases during the incubation (for example, during an amplification reaction), providing a signal change. In case of FIG. 1d and FIG. 1e, the single-typed duplex has initially been included in the composition for detecting a target nucleic acid, and the amount of the single-typed duplex decreases during the incubation reaction, thereby providing a signal change.

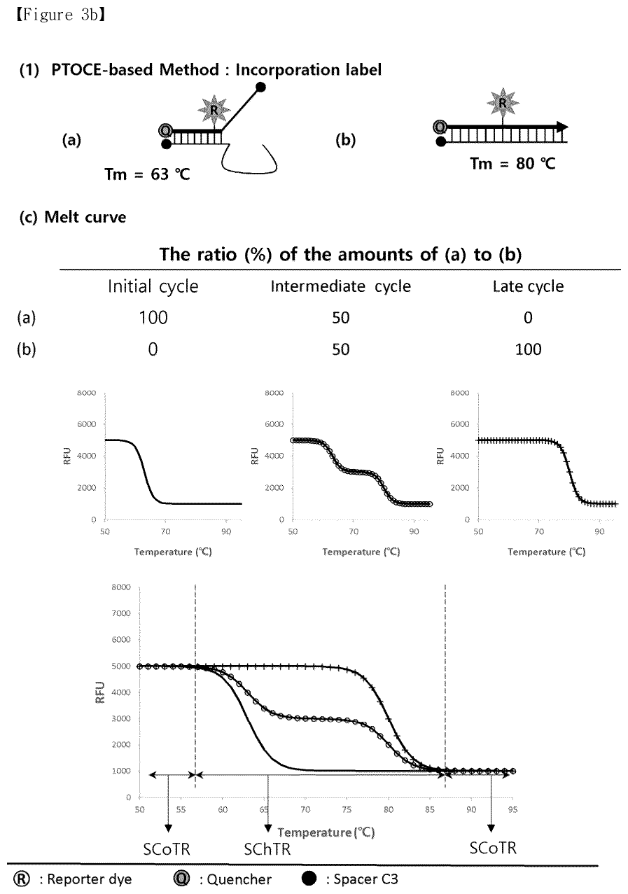

In one embodiment, when the duplex is plural-typed duplexes, the amount ratio between the plural-typed duplexes changes depending on the presence of a target nucleic acid, thereby changing the signal. For example, with reference to the drawings, the PTOCE-based method in FIG. 3a and FIG. 3b is an example of providing plural-typed duplexes (in particular, two types of duplexes). In FIG. 3a and FIG. 3b, one of the two types of duplexes has initially been included in the composition for detecting the target nucleic acid, and the other one is generated during an incubation reaction, and during the incubation reaction (e.g., during an amplification reaction) the amount ratio between the two types of duplexes changes, thereby providing a signal change. In particular, as the amount of a duplex initially included in the composition for detecting the target nucleic acid decreases, and the amount of a duplex newly generated during the incubation reaction increases, the amount ratio between these duplexes changes.

In one embodiment, at least two of the plural-typed duplexes include the same single-strand. For example, with reference to FIG. 3a and FIG. 3b, in the two different types of duplexes, one of two single-strands constituting each duplex is the same strand. In particular, a CTO and a PTO (that is, uncleaved PTO), wherein the PTO (or CTO) has interactive dual labels linked thereto, or the PTO has one of interactive dual labels and the CTO has the other of the interactive dual labels, constitute a first duplex providing a signal change and has initially been included in the composition for detecting the target nucleic acid, and during an incubation reaction, the PTO is cleaved depending on the presence of the target nucleic acid, to release a fragment, and the released fragment is hybridized to a CTO, to generate an extended strand, and the newly-generated extended strand and the CTO constitute a second duplex providing a signal change. Here, the first duplex and the second duplex include the same single-strand (that is, a CTO).

In one embodiment, Tm values of the plural-typed duplexes are different from each other. For example, the Tm values of the duplexes are different from each other at least by 2° C., 3° C., 4° C., 5° C., 7° C., 8° C., 9° C., 10° C., 11° C., 12° C., 13° C., 14° C., 15° C., or 20° C.

In one embodiment, the amount of the duplex refers to the sum of the amount of a duplex in a state in which two nucleic acid strands constituting the duplex are dissociated (that is, duplex in dissociated form) and the amount of a duplex in a state in which the two nucleic acid strands are hybridized (that is, duplex in associated form).

In one embodiment, when the plural-typed duplexes include the same single-strand, the same single-strand is included in a first duplex initially included in the composition for detecting the target nucleic acid, and during an incubation reaction, a new second duplex including the same single-strand may be generated. In this case, it may be considered that the same single-strand included in the first duplex is consumed while generating the second duplex during the incubation reaction, and as a result, the amount of the first duplex including the same single-strand decreases, and the amount of the second duplex including the same single-strand increases.

In one embodiment, any one of the n compositions for detecting the target nucleic acids may amplify a corresponding target nucleic acid.

In one embodiment, any one of the n compositions for detecting the target nucleic acids may include an amplification oligonucleotide that serves to amplify a corresponding target nucleic acid. In one embodiment, the amplification oligonucleotide may be the same as the label-linked oligonucleotide.

As used herein, the term “amplification oligonucleotide” refers to any oligonucleotides that serve to amplify target nucleic acids.

In one embodiment, the amplification oligonucleotide may be a ‘primer’ known in the art. As used herein, the term ‘primer’ refers to an oligonucleotide that is capable of acting as a point of initiation of synthesis when placed under conditions in which synthesis of primer extension product which is complementary to a target nucleic acid strand (template) is induced, i.e., in the presence of nucleotides and an agent for polymerization, such as DNA polymerase, and at a suitable temperature and pH. The primer must be long enough to prime the synthesis of extension products in the presence of the agent for polymerization. An appropriate length of the primer is determined by multiple factors, including temperature, the field of application, and the source of primer.

The primer may include a forward primer (also referred to as an upstream primer or an upstream oligonucleotide), a reverse primer (also referred to as a downstream primer or a downstream oligonucleotide), or both. The amplification oligonucleotide may be an oligonucleotide having a structure known in the art, or may be synthesized by a method known in the art.

That the amplification oligonucleotide and the label-linked oligonucleotide are the same means that a single oligonucleotide simultaneously acts as an amplification oligonucleotide that amplifies a target nucleic acid, and as a label-linked oligonucleotide that generates a signal in the presence of the target nucleic acid. As one example, the label-linked oligonucleotide may be hybridized to the target nucleic acid and extended, thereby generating a signal.

The present inventors have found that various signal generation mechanisms using a duplex involve a temperature range in which the signal changes depending on the presence of the target nucleic acid (that is, a signal-changing temperature range), and a temperature range in which the signal does not change even in the presence of the target nucleic acid (that is, a signal-constant temperature range). Further, the present inventors have found that methods not providing a duplex providing a signal, such as a TaqMan probe assay, do not have a signal-constant temperature range.

Further, the present inventors have found that well-known signal generation mechanisms can be classified into three types depending on the number and/or order of the signal-changing temperature range and the signal-constant temperature range, and by using various combinations of these three types of signal generation mechanism, have developed a novel method that is capable of detecting multiple target nucleic acids in a real-time manner utilizing a single-typed label, without post amplification melting analysis.

In one embodiment, in the temperature range covering all of the n detection temperatures, the composition for detecting the i^{th} target nucleic acid has a “signal-changing temperature range” in which the signal changes depending on the presence of the i^{th} target nucleic acid, and a “signal-constant temperature range” in which the signal is constant even in the presence of the i^{th} target nucleic acid.

In one embodiment, among the n compositions for detecting target nucleic acids, the composition for detecting the i^{th} target nucleic acid may have a “signal-changing temperature range” in which the signal changes as the target nucleic acid is amplified, and a “signal-constant temperature range” in which the signal is constant even as the target nucleic acid is amplified.

In one embodiment, the signal-changing temperature range is a temperature range in which a difference in signal value (e.g., signal intensity) is generated depending on the presence of the target nucleic acid.

In one embodiment, the signal-changing temperature range is a temperature range in which the signal value changes depending on the level of amplification of the target nucleic acid (e.g., the amount of the amplified target nucleic acid).

In one embodiment, the signal-constant temperature range is a temperature range in which the signal value does not change regardless of the presence of the target nucleic acid. That is, the signal-constant temperature range is a temperature range in which there is no difference between the signal value in the presence of the target nucleic acid, and the signal value in the absence of the target nucleic acid.

In one embodiment, the composition for detecting the i^{th} target nucleic acid may have the signal-changing temperature range and the signal-constant temperature range in a continuous or discontinuous order.

In one embodiment, the composition for detecting the i^{th} target nucleic acid may have one or two signal-constant temperature ranges.

In one embodiment, the i^{th} detection temperature may be selected from within the signal-changing temperature range of the composition for detecting the i^{th} target nucleic acid. In this case, in the present application, the composition for detecting the i^{th} target nucleic acid is referred to as having the i^{th} detection temperature. In addition, the i^{th} target nucleic acid corresponding to the composition for detecting the i^{th} target nucleic acid may be referred to as a target nucleic acid having the i^{th} detection temperature.

According to an embodiment, one detection temperature, which is determined by the composition for detecting a corresponding target nucleic acid, is assigned to one target nucleic acid.

In one embodiment, the signal-changing temperature ranges of the n compositions for detecting target nucleic acids do not overlap with each other. In this case, for each of the n detection temperatures, any temperature may be selected as a detection temperature as long as it is within a corresponding signal-changing temperature range.

In one embodiment, the i^{th} detection temperature is selected from within the signal-changing temperature range of the composition for detecting the i^{th} target nucleic acid, and the i^{th} detection temperature is not included in the signal-changing temperature ranges of the compositions for detecting the other target nucleic acids.

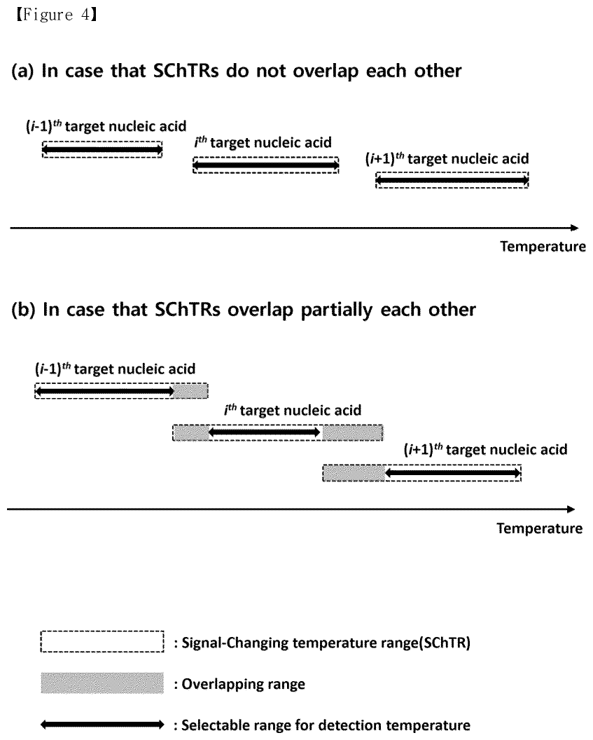

In one embodiment, the signal-changing temperature range of any one composition among the compositions for detecting target nucleic acids may overlap with the signal-changing temperature range of a composition for detecting a target nucleic acid having an adjacent detection temperature, while not overlapping with the signal-changing temperature range of a composition for detecting a target nucleic acid having a detection temperature that is not adjacent thereto. In this case, the detection temperature of the composition for detecting a target nucleic acid that has the signal-changing temperature range overlapping with the signal-changing temperature range of the composition for detecting another target nucleic acid is selected within a temperature range of the signal-changing temperature range that does not overlap with the signal-changing temperature range of the composition for detecting the another target nucleic acid. As such, by selecting the detection temperature from within a temperature range that does not overlap between the two signal-changing temperature ranges, only the signal indicative of the presence of a single particular target nucleic acid can be provided at a single detection temperature (see FIG. 4).

In one embodiment, the signal-changing temperature range of any one of compositions for detecting target nucleic acids may overlap with the signal-changing temperature range of the composition for detecting a target nucleic acid having an adjacent detection temperature, but either one of the two signal-changing temperature ranges is not completely included in the other signal-changing temperature range.

The term “adjacent detection temperature” is used herein to refer to consecutive detection temperatures among n detection temperatures, and for example, the adjacent detection temperature of the i^{th} detection temperature is the (i−1)^{th} detection temperature or the (i+1)^{th} detection temperature.

In one embodiment, the signal-changing temperature range of the composition for detecting the i^{th} target nucleic acid may partially overlap with the signal-changing temperature range of the composition for detecting a target nucleic acid having an adjacent detection temperature, while not overlapping with the signal-changing temperature range of the composition for detecting a target nucleic acid having a detection temperature that is not adjacent thereto.

In one embodiment, the composition for detecting the i^{th} target nucleic acid may have one signal-changing temperature range and one signal-constant temperature range. In one embodiment, the composition for detecting the i^{th} target nucleic acid may have one signal-changing temperature range and two signal-constant temperature ranges.

In one embodiment, the composition for detecting the i^{th} target nucleic acid may be any one of: (i) an Under-Signal-Change (UnderSC) composition having a characteristic that the signal-changing temperature range is lower than the signal-constant temperature range; (ii) an Over-Signal-Change (OverSC) composition having a characteristic that the signal-changing temperature range is higher than the signal-constant temperature range; and (iii) an Inter-Signal-Change (InterSC) composition having a characteristic that the signal-changing temperature range is higher than one of two signal-constant temperature ranges, and lower than the other of the two signal-constant temperature ranges.

In one embodiment, the expression “one temperature range is lower than the other temperature range” used with regard to the signal-changing temperature range and signal-constant temperature range of the composition for detecting a target nucleic acid means that the highest temperature of one temperature range is lower than the lowest temperature of the other temperature range. Further, the expression “one temperature range is higher than the other temperature range” means that the lowest temperature of one temperature range is higher than the highest temperature of the other temperature range. For example, that a signal-constant temperature range is higher than a signal-changing temperature range means that the lowest temperature within the signal-constant temperature range is higher than the highest temperature within the signal-changing temperature range.

In one embodiment, the signal-changing temperature range of the composition for detecting the i^{th} target nucleic acid may be determined depending on the length and/or sequence of the duplex providing a signal change.

In one embodiment, the composition for detecting the i^{th} target nucleic acid provides a single-typed duplex, the composition for detecting a target nucleic acid may have one signal-changing temperature range and one signal-constant temperature range. The signal-changing temperature range and signal-constant temperature range may be determined depending on the length and/or sequence of the single-typed duplex.

In one embodiment, when the composition for detecting the i^{th} target nucleic acid provides plural-typed duplexes, in particular, two types of duplexes, the composition for detecting a target nucleic acid may have one signal-changing temperature range and two signal-constant temperature ranges. The signal-changing temperature range and the signal-constant temperature ranges may be determined depending on the lengths and/or sequences of the two types of duplexes.

Any one of the n compositions for detecting target nucleic acids used herein may employ various signal generation mechanisms described above.

In one embodiment, the above-described various signal generation mechanisms may be used for any one of an UnderSC composition, an InterSC composition, and an OverSC composition.

In one embodiment, even when the signal generation mechanism of the composition for detecting a target nucleic acid is the same, the compositions for detecting target nucleic acids including oligonucleotides of different sequences may be considered different from each other. The different compositions for detecting nucleic acids have different detection temperatures from each other.

More specifically, with reference to the drawings, examples of signal generation mechanisms adoptable by an UnderSC composition, an InterSC composition, and an OverSC composition, are described, but are not limited thereto.

### (i) Signal Generation Mechanism Adoptable by an UnderSC Composition for Detecting a Target Nucleic Acid

FIG. 1a to FIG. 1h show various signal generation mechanisms adoptable by an UnderSC composition.

The signal generation mechanisms in FIG. 1a and FIG. 1h have one signal-changing temperature range in which the signal changes upon a target nucleic acid amplification, and one signal-constant temperature range in which the signal is constant even as the target nucleic acid is amplified, wherein the signal-changing temperature range is lower than the signal-constant temperature range.

In one embodiment, as in the molecular beacon method in FIG. 1a, the LUX method in FIG. 1b, and the hybridization probe method in FIG. 1c, various signal generation mechanisms that provide a signal change from a duplex (that is, a duplex providing a signal change) by hybridization between an oligonucleotide linked with a label (e.g., a single label or interactive labels) and a target nucleic acid may be utilized for an UnderSC composition for detecting a target nucleic acid. Here, the duplexes providing signal changes are duplexes generated during the incubation reaction.

In one embodiment, as the signal generation mechanism for the UnderSC composition for detecting a target nucleic acid, various signal generation mechanisms such as those shown in the Yin-Yang probe method in FIG. 1d and the PCE-NH method in FIG. 1e, may be utilized, in which the composition for detecting a target nucleic acid initially includes a duplex providing a signal change, and one of the two single-strands constituting the duplex providing the signal change pairs with the target nucleic acid, thereby forming a new duplex, or a new duplex is formed as one of the two single-strands constituting the duplex providing the signal change pairs with one of the two strands constituting the duplex formed by a cleavage reaction dependent on hybridization of the target nucleic acid and a mediating oligonucleotide, such that the amount of the duplex providing the signal change changes (in particular, decreases), thereby providing a change in signal.

In one embodiment, various signal generation mechanisms such as the ones shown in the PTOCE-based method in FIG. 1f, FIG. 1g, and FIG. 1h, in which a signal change is provided from a duplex providing a signal change, formed by a cleavage reaction dependent on hybridization of a mediating oligonucleotide and the target nucleic acid, may be used for the UnderSC composition. Here, the duplexes providing the signal changes are duplexes generated during an incubation reaction, and in particular, the label of the duplex providing a signal change in FIG. 1f (that is, an extended duplex) is incorporated during an incubation reaction.

### (ii) Signal Generation Mechanism Adoptable by an OverSC Composition for Detecting a Target Nucleic Acid

FIG. 2a to FIG. 2c show various signal generation mechanisms adoptable by an OverSC composition.

The signal generation mechanisms in FIG. 2a and FIG. 2c have one signal-changing temperature range in which the signal changes upon a target nucleic acid amplification, and one signal-constant temperature range in which the signal is constant even as the target nucleic acid is amplified, wherein the signal-changing temperature range is higher than the signal-constant temperature range.

In one embodiment, various signal generation mechanisms such as the ones shown in the PTOCE-based methods in FIG. 2a and FIG. 2c, and the dual quenching method in FIG. 2b, in which a signal change is provided from a duplex providing a signal change, formed by a cleavage reaction dependent on hybridization of a mediating oligonucleotide and the target nucleic acid, may be used for the UnderSC composition. Here, the duplexes providing the signal changes are duplexes generated during an incubation reaction, and in particular, the label of the duplex providing a signal change in FIG. 2c (that is, an extended duplex) is incorporated during an incubation reaction.

(iii) Signal Generation Mechanism Adoptable by an InterSC Composition for Detecting a Target Nucleic Acid

FIG. 3a to FIG. 3b show various signal generation mechanisms adoptable by an InterSC composition.

The signal generation mechanisms in FIG. 3a and FIG. 3b have one signal-changing temperature range in which the signal changes as the target nucleic acid is amplified, and two signal-constant temperature ranges in which the signal is constant even as the target nucleic acid is amplified, wherein the signal-changing temperature range is higher than one of the two signal-constant temperature ranges, and lower than the other of the two signal-constant temperature ranges.

In one embodiment, various signal generation mechanisms providing a signal change from a duplex providing multiple types of signal change, may be used for the InterSC composition.

For example, as in the PTOCE-based methods shown in FIG. 3a and FIG. 3b, the mechanism in which a signal change is provided from two types of duplexes providing the signal change may be used, and in particular, in the methods in FIG. 3a and FIG. 3b, one of the two types of duplexes has initially been included in the composition for detecting a target nucleic acid, and the other one is generated by a cleavage reaction dependent on hybridization of a mediation oligonucleotide and the target nucleic acid, and as an incubation reaction progresses, the amount ratio between these two types of duplexes changes, thereby providing a signal change. Here, the two types of duplexes have different Tm values from each other.

In one embodiment, the plurality of duplexes may have different Tm values from each another.

In one embodiment, the InterSC composition has one signal-changing temperature range in which the signal changes depending on the presence of the target nucleic acid, and two signal-constant temperature ranges in which the signal is constant even in the presence of the target nucleic acid, wherein the signal-changing temperature range is higher than one of the two signal-constant temperature ranges, and lower than the other of the two signal-constant temperature ranges.

In one embodiment, the InterSC composition provides plural-typed duplexes providing a signal change.

In one embodiment, the plural-typed duplexes have different Tm values from each another.

In one embodiment, the signal-changing temperature range and the signal-constant temperature ranges of the InterSC composition may be adjusted by adjusting the Tm values of the plural-typed duplexes, and the target nucleic acid may be detected using a detection temperature selected from the signal-change temperature range.

In one embodiment, the amount ratio between the plural-typed duplexes changes depending on the presence of the target nucleic acid, thereby changing the signal.

In one embodiment, the InterSC composition provides two duplexes providing a signal change. Depending on the presence of the target nucleic acid, the amount ratio between the two duplexes is changed, resulting in the signal change. The two duplexes have different Tm values.

In one embodiment, one (e.g., a duplex with a relatively low Tm), of the two duplexes providing the signal change has initially been included in the InterSC composition, and the other one (e.g., a duplex with a relatively high Tm) is generated during an incubation reaction.

In one embodiment, one of two single-strands constituting each duplex in the two duplexes is the same strand. In particular, as the amount of a duplex initially included in the composition for detecting the target nucleic acid decreases, and the amount of a duplex newly generated during the incubation reaction increases, the amount ratio between these duplexes changes. That is, the amount of the first duplex that has initially been included in the InterSC composition, of the two duplexes comprising the same strand, is decreased by the second duplex newly generated during the incubation reaction, and during the incubation, the amount of the second duplex is increased, and the amount ratio between them changes.

In one embodiment, any one of the duplexes provided by the InterSC composition is a duplex provided by a cleavage reaction dependent on the presence of the target nucleic acid. For example, a duplex which is newly generated during the incubation reaction, may be generated by a cleavage reaction (e.g., PTOCE-based method) dependent on the presence of the target nucleic acid.

In one embodiment, the two duplexes each comprise interactive dual labels (e.g., a reporter molecule and a quencher molecule). In particular, (i) when the two duplexes are in associated form, the quencher molecule is in close proximity to the reporter molecule, thereby quenching the signal from the reporter molecule, and when the two duplexes are in dissociated form, the quencher molecule is separated from the reporter molecule, thereby unquenching the signal from the reporter molecule (see FIG. 3a), or (ii) when the two duplexes are in associated form, the quencher molecule is separated from the reporter molecule, thereby unquenching the signal from the reporter molecule, and when the two duplexes are in dissociated form, the quencher molecule is in close proximity to the reporter molecule, thereby quenching the signal from the reporter molecule. (see FIG. 3b)

Signal generation mechanisms adoptable by an InterSC composition can be used to detect a single target nucleic acid as well as be used to detect multiple target nucleic acids by using a plurality of InterSC compositions and adjusting each signal-changing temperature range of the plurality of InterSC compositions.

As described above, the compositions for detecting target nucleic acids, even when their signal generation mechanism is the same, may have a different order of the signal-changing temperature range and signal-constant temperature range, depending on (i) the type of label (e.g., a single label, interactive labels, etc.), (ii) the type of a label-linked oligonucleotide (e.g., PTO, CTO, etc.), and (iii) the location at which a label is linked and/or the manner by which a label is linked (whether the label is initially linked to an oligonucleotide prior to the incubation reaction, or linked to an oligonucleotide by being incorporated therein during the incubation reaction, etc.), and the like.

Accordingly, the same signal generation mechanism may be used for any one of the UnderSC composition, the InterSC composition, and the OverSC composition. For example, in the PTOCE-based method, the mechanism by which the presence of a target nucleic acid is determined by a signal from an extension and cleavage of a PTO may be the same, but the case in which interactive dual labels are all linked to a CTO (e.g., FIG. 1g, FIG. 1h) and the case in which one of interactive labels is linked to PTO and the other of the interactive labels is linked to CTO (e.g., FIG. 3a) may be applied as the signal generation mechanism for the UnderSC composition and the InterSC composition, respectively. In addition, the case in which the interactive dual labels are incorporated (e.g., FIG. 2c) and the case in which it is initially linked to an oligonucleotide (e.g., FIG. 3b) may be applied as the signal generation mechanisms for the OverSC composition and the InterSC composition for detecting a target nucleic acid, respectively.

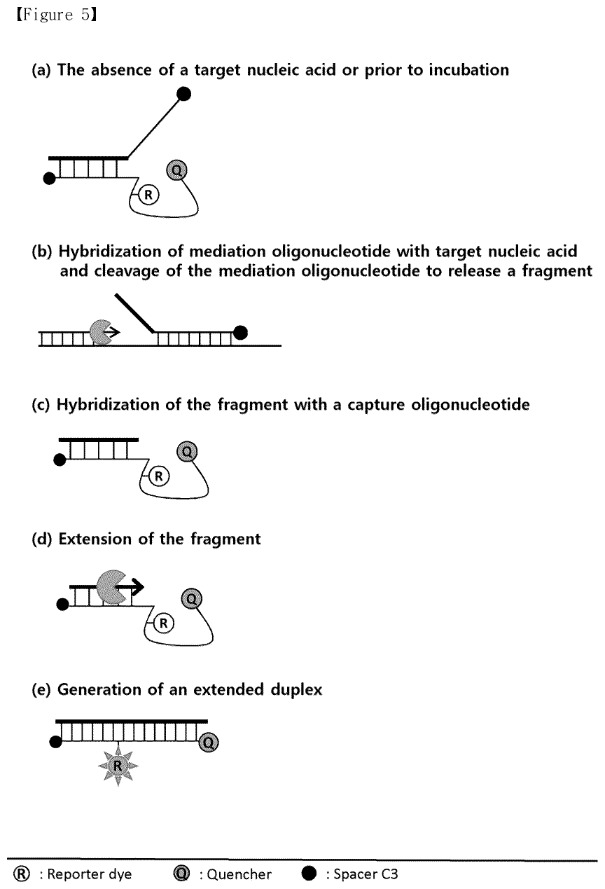

In a particular embodiment, as in the above-described PTOCE-based method, specifically, the PTOCE-based method in which the CTO having interactive dual labels linked thereto forms a duplex with an extended strand of a PTO fragment cleaved depending on the presence of the target nucleic acid, thereby providing a signal, may be adopted as the signal generation mechanism for the UnderSC composition (see FIG. 5). Here, the signal-changing temperature range and signal-constant temperature range of the UnderSC composition may be controlled by adjusting the Tm value of the extended duplex (that is, a duplex between the interactive dual label-linked CTO and the extended strand).

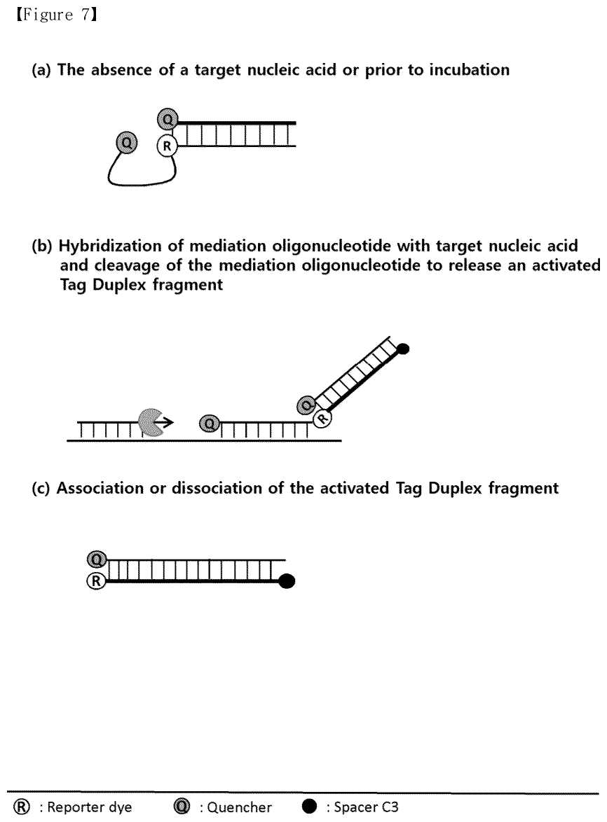

In a particular embodiment, the above-described dual quenching method may be adopted as the signal generation mechanism for the OverSC composition (see FIG. 7). Here, the signal-changing temperature range and signal-constant temperature range of the OverSC composition may be controlled by adjusting the Tm value of the tag duplex (that is, an activated tag duplex fragment).

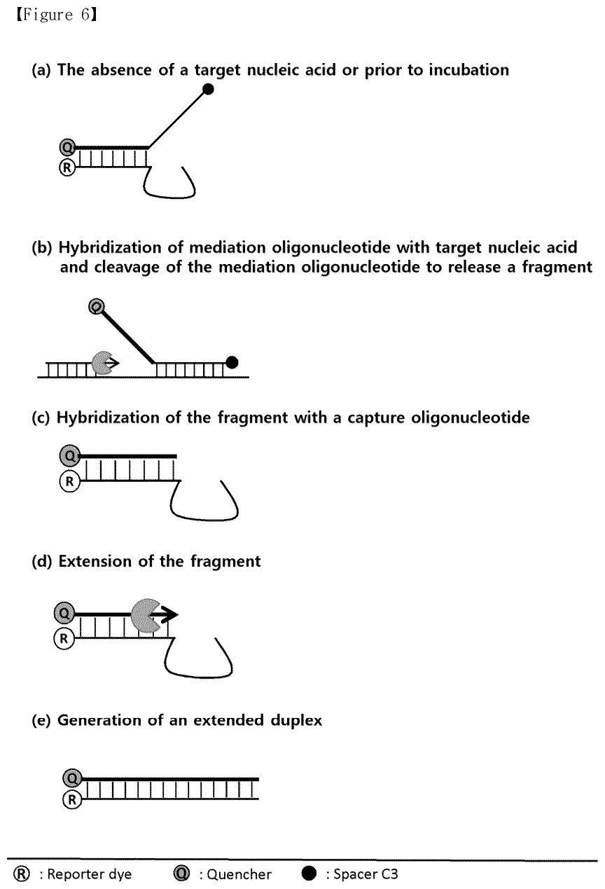

In a particular embodiment, the above-described PTOCE-based method, specifically, the PTOCE-based method using a PTO having one of interactive dual labels linked thereto, and a CTO having the other one of the interactive dual label linked thereto, may be adopted as the signal generation mechanism for the InterSC composition (see FIG. 6). Here, the signal-changing temperature range and signal-constant temperature range of the InterSC composition may be controlled by adjusting the Tm value of a duplex between a CTO and a tagging portion of uncleaved PTO, and the Tm value of an extended duplex between CTO and an extended strand of a PTO fragment cleaved dependently on the presence of a target nucleic acid.

In one embodiment, when n is 2, the composition for detecting a first target nucleic acid may be an UnderSC composition or an InterSC composition, and the composition for detecting a second target nucleic acid may be an InterSC composition or an OverSC composition.

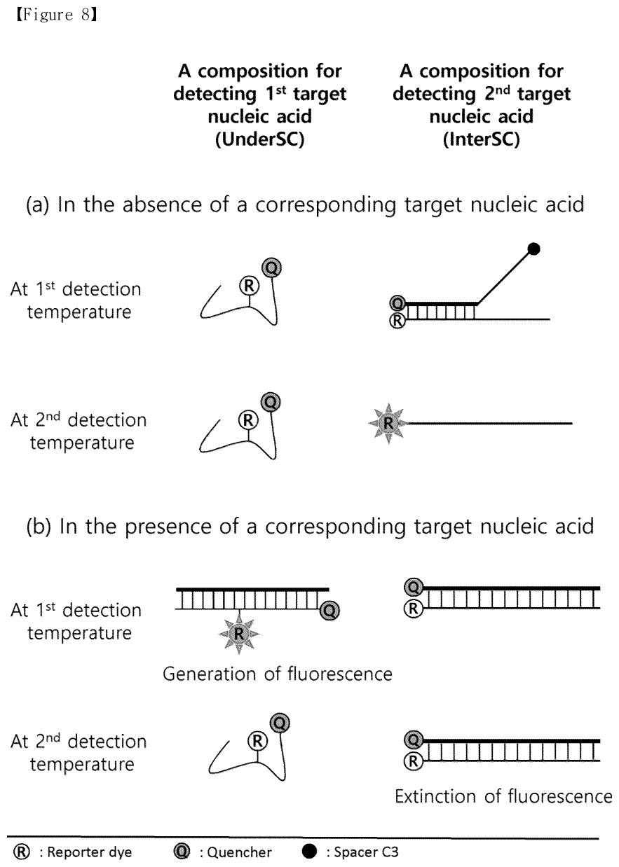

In a particular embodiment, when n is 2, the composition for detecting the first target nucleic acid may be an UnderSC composition and the composition for detecting the second target nucleic acid may be an InterSC composition. For example, as shown in FIG. 8, for the signal generation mechanism of the composition for detecting the first target nucleic acid, the PTOCE-based method which uses a CTO linked with interactive dual labels may be used, and for the signal generation mechanism of the composition for detecting the second target nucleic acid, the PTOCE-based method in which one of interactive dual labels is linked to a PTO, and the other is linked to a CTO, may be used. In one embodiment, the first detection temperature may be selected to be a temperature at which a duplex provided by the composition for detecting the first target nucleic acid (that is, an extended duplex) and two types of duplexes provided by the composition for detecting the second target nucleic acid (that is, a duplex between uncleaved PTO and CTO, and an extended duplex) are all in associated form, and the second detection temperature may be selected to be a temperature at which the extended duplex provided by the composition for detecting the first target nucleic acid and the duplex between uncleaved PTO and CTO, provided by the composition for detecting the second target nucleic acid are in dissociated form, and at which the extended duplex provided by the composition for detecting the second target nucleic acid is in associated form. The composition for detecting the first target nucleic acid, in the presence of the first target nucleic acid, provides a signal change at the first detection temperature (that is, as an extended duplex in associated form is generated upon target nucleic acid amplification, such that the quencher molecule is separated from the reporter molecule, thereby unquenching the signal from the reporter molecule), and provides a constant signal at the second detection temperature (that is, the extended duplex, even when generated, is all in dissociated form such that the quencher molecule is in close proximity to the reporter molecule, thereby quenching the signal from the reporter molecule), and the composition for detecting the second target nucleic acid, in the presence of the second target nucleic acid, provides a constant signal at the first detection temperature (that is, the two types of duplexes are both in associated form such that the quencher molecule is in close proximity to the reporter molecule, thereby quenching the signal from the reporter molecule) and provides a signal change at the second detection temperature (the duplex between uncleaved PTO and CTO is consumed, and an extended duplex in associated form is generated, such that the quencher molecule is in close proximity to the reporter molecule, thereby quenching the signal from the reporter molecule).

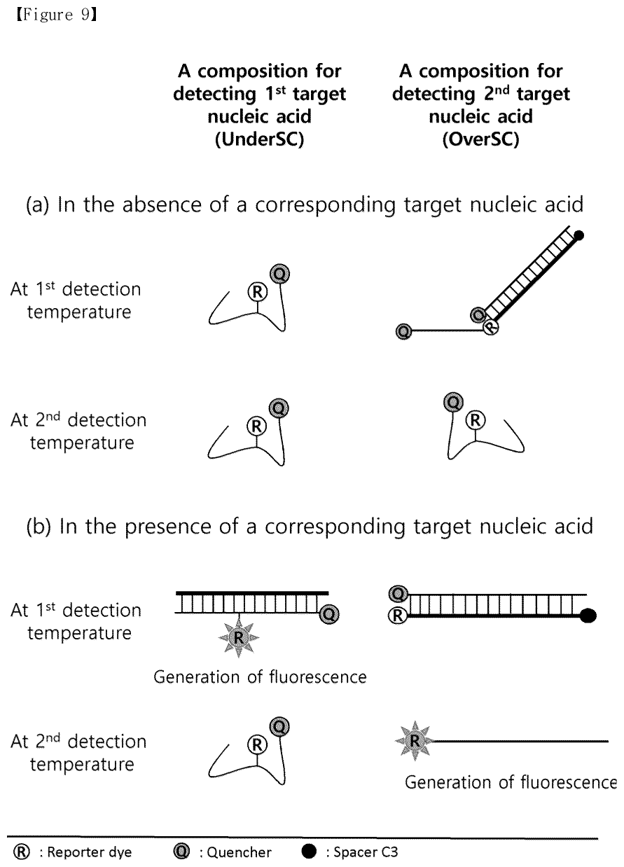

In a particular embodiment, when n is 2, the composition for detecting the first target nucleic acid may be an UnderSC composition and the composition for detecting the second target nucleic acid may be an OverSC composition. For example, as shown in FIG. 9, for the signal generation mechanism of the composition for detecting the first target nucleic acid, the PTOCE-based method using a CTO linked with interactive dual labels may be used, and for the signal generation mechanism of the composition for detecting the second target nucleic acid, the dual-quenching method may be used. In one embodiment, the first detection temperature may be selected to be a temperature at which a duplex provided by the composition for detecting a first target nucleic acid (that is, an extended duplex) and a duplex provided by the composition for detecting a second target nucleic acid (that is, a tag duplex) are in associated form, and the second detection temperature may be selected to be a temperature at which the extended duplex and the tag duplex are both in dissociated form. In this case, the composition for detecting a first target nucleic acid, in the presence of the first target nucleic acid, provides a signal change at the first detection temperature (that is, as an extended duplex in associated form is generated upon target nucleic acid amplification, such that quencher molecule is separated from the reporter molecule, thereby unquenching the signal from the reporter molecule) and provides a constant signal at the second detection temperature (that is, the extended duplex, even when generated, is all in dissociated form such that the quencher molecule is in close proximity to the reporter molecule, thereby quenching the signal from the reporter molecule); the composition for detecting a second target nucleic acid, in the presence of the second target nucleic acid, provides a constant signal at the first detection temperature (that is, in a tag duplex in associated form (an uncleaved tag duplex and an activated tag duplex fragment), at least one of two quenchers is in close proximity to the reporter molecule, thereby quenching the signal from the reporter molecule) and provides a signal change at the second detection temperature (that is, an activated tag duplex fragment generated upon target nucleic acid amplification is in dissociated form, such that both of the two quencher molecules are separated from the reporter molecule, thereby unquenching the signal from the reporter molecule).

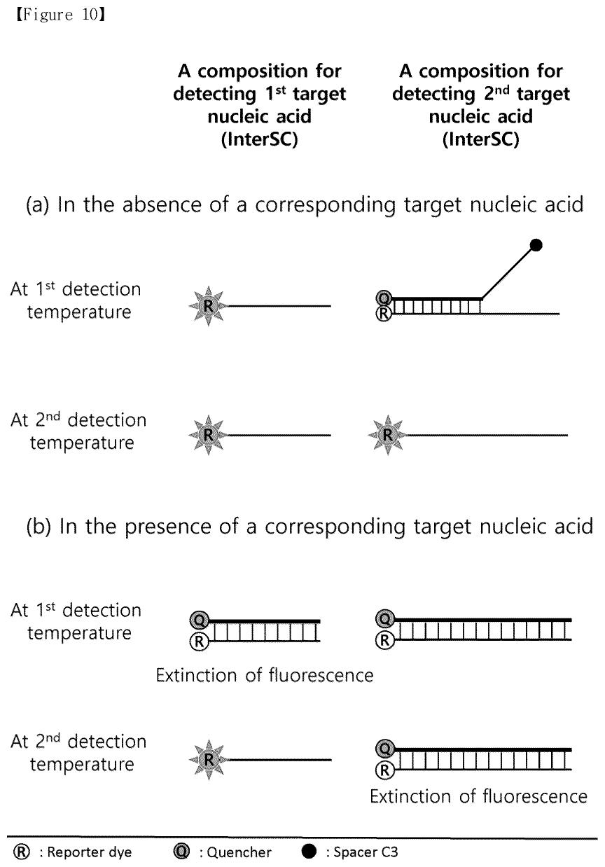

In a particular embodiment, when n is 2, the composition for detecting the first target nucleic acid may be an InterSC composition and the composition for detecting the second target nucleic acid may be an InterSC composition. For example, as shown in FIG. 10, as the signal generation mechanism of the composition for detecting the first target nucleic acid and the composition for detecting the second target nucleic acid, the PTOCE-based method in which one of interactive dual labels is linked to a PTO, and the other to a CTO, may be used. In one embodiment, the first detection temperature may be selected to be a temperature at which, among the two types of duplexes provided by the composition for detecting the first target nucleic acid (that is, an extended duplex and a duplex between uncleaved PTO and CTO) and the two types of duplexes provided by the composition for detecting the second target nucleic acid (that is, an extended duplex and a duplex between uncleaved PTO and CTO), all the other duplexes, except the duplex between uncleaved PTO and CTO provided by the composition for detecting the first target nucleic acid, are in associated form, and the duplex between uncleaved PTO and CTO provided by the composition for detecting the first target nucleic acid is in dissociated form; and the second detection temperature may be selected to be a temperature at which, among the two types of duplexes provided by the composition for detecting the first target nucleic acid and the two types of duplexes provided by the composition for detecting the second target nucleic acid, only the extended duplex provided by the composition for detecting the second target nucleic acid is in associated form, and the rest of the other duplexes are all in dissociated form. The composition for detecting the first target nucleic acid, in the presence of the first target nucleic acid, provides a signal change at the first detection temperature (that is, as an extended duplex in associated form is generated upon target nucleic acid amplification, the quencher molecule is in close proximity to the reporter molecule, thereby quenching the signal from the reporter molecule), and provides a constant signal at the second detection temperature (that is, the extended duplex, even when generated, is all in dissociated form such that the quencher molecule is separated from the reporter molecule, thereby unquenching the signal from the reporter molecule); and the composition for detecting the second target nucleic acid, in the presence of the second target nucleic acid, provides a constant signal at the first detection temperature (that is, the two types of duplexes are both in associated form such that the quencher molecule is in close proximity to the reporter molecule, thereby quenching the signal from the reporter molecule) and provides a signal change at the second detection temperature (as the duplex between uncleaved PTO and CTO is consumed, and an extended duplex in associated form is generated, the quencher molecule is in close proximity to the reporter molecule, thereby quenching the signal from the reporter molecule).

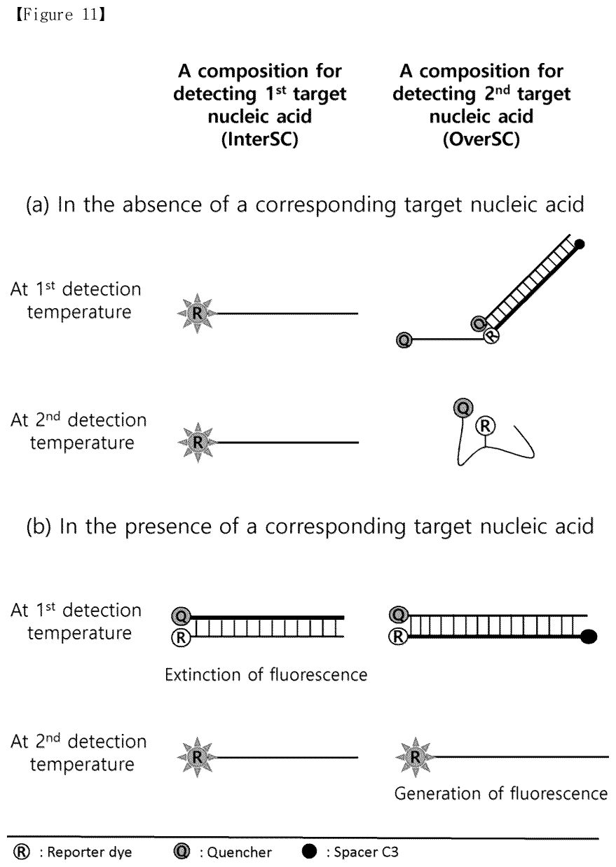

In a particular embodiment, when n is 2, the composition for detecting the first target nucleic acid may be an InterSC composition and the composition for detecting the second target nucleic acid may be an OverSC composition. For example, as shown in FIG. 11, for the signal generation mechanism of the composition for detecting the first target nucleic acid, the PTOCE-based method in which one of dual labels is linked to PTO and the other to CTO, may be used, and for the signal generation mechanism of the composition for detecting the second target nucleic acid, the dual-quenching method may be used. In one embodiment, the first detection temperature may be selected to be a temperature at which, among the two types of duplexes provided by the composition for detecting the first target nucleic acid (that is, an extended duplex and a duplex between uncleaved PTO and CTO) and an activated tag duplex fragment provided by the composition for detecting the second target nucleic acid, all the other duplexes, except the duplex between uncleaved PTO and CTO provided by the composition for detecting the first target nucleic acid, are all in associated form, and the duplex between uncleaved PTO and CTO provided by the composition for detecting the first target nucleic acid is in dissociated form; and the second detection temperature may be selected to be a temperature at which the two types of duplexes provided by the composition for detecting the first target nucleic acid (an extended duplex and a duplex between uncleaved PTO and CTO) and the activated tag duplex fragment provided by the composition for detecting the second target nucleic acid are all in dissociated form. In this case, the composition for detecting a first target nucleic acid, in the presence of the first target nucleic acid, provides a signal change at the first detection temperature (that is, as an extended duplex in associated form is generated upon target nucleic acid amplification, the quencher molecule is in close proximity to the reporter molecule, thereby quenching the signal from the reporter molecule) and provides a constant signal at the second detection temperature (that is, the extended duplex, even when generated, is all in dissociated form, such that the quencher molecule is separated from the reporter molecule, thereby unquenching the signal from the reporter molecule); the composition for detecting a second target nucleic acid, in the presence of the second target nucleic acid, provides a constant signal at the first detection temperature (that is, in a tag duplex in associated form (an uncleaved tag duplex and an activated tag duplex fragment), at least one of two quenchers is in close proximity to the reporter molecule, thereby quenching the signal from the reporter molecule) and provides a signal change at the second detection temperature (that is, an activated tag duplex fragment generated upon target nucleic acid amplification is in dissociated form, such that both of the two quencher molecules are separated from the reporter molecule, thereby unquenching the signal from the reporter molecule).

In one embodiment, when n is 3 or more, the composition for detecting the first target nucleic acid may be an UnderSC composition or an InterSC composition, the composition for detecting an n^{th} target nucleic acid may be an InterSC composition or an OverSC composition, and the composition(s) for detecting target nucleic acids, other than the first target nucleic acid and the n^{th} target nucleic acid, may be an InterSC composition.

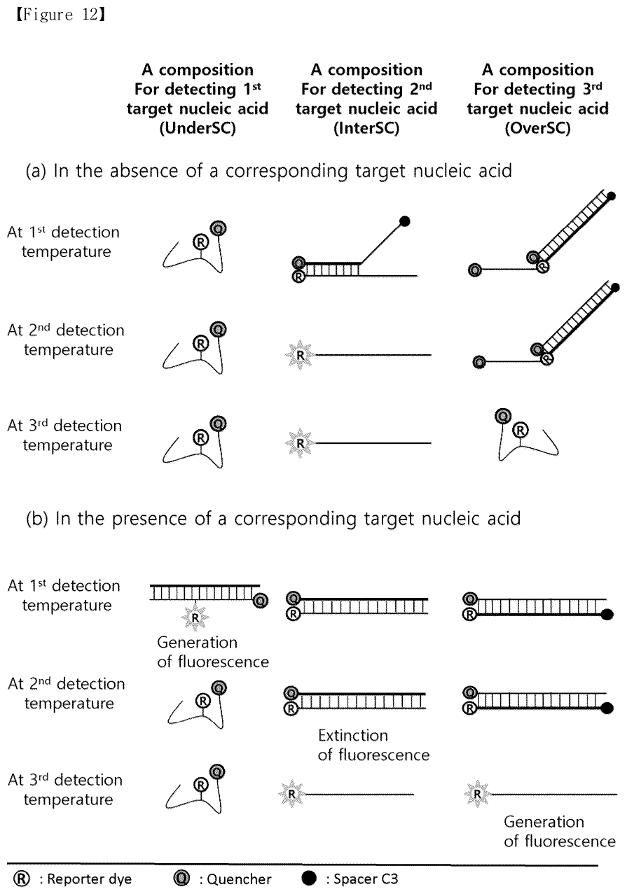

In a particular embodiment, when n is 3, the composition for detecting a first target nucleic acid may be an UnderSC composition, the composition for detecting a second target nucleic acid may be an InterSC composition, and the composition for detecting a third target nucleic acid may be an OverSC composition. For example, as shown in FIG. 12, for the signal generation mechanism of the composition for detecting the first target nucleic acid, the PTOCE-based method which uses a CTO linked with interactive dual labels may be used, and for the signal generation mechanism of the composition for detecting the second target nucleic acid, the PTOCE-based method in which one of interactive dual labels is linked to a PTO, and the other is linked to a CTO, may be used, and for the signal generation mechanism of the composition for detecting the third target nucleic acid, the dual-quenching method may be used. In one embodiment, the first detection temperature may be selected to be a temperature at which duplexes (In particular, duplexes providing the signal changes) provided by the compositions for detecting the first to third target nucleic acids are all in associated form, the second detection temperature may be selected to be a temperature at which the extended duplex provided by the composition for detecting the first target nucleic acid, and the duplex between uncleaved PTO and CTO, provided by the composition for detecting the second target nucleic acid, are in dissociated form, and the other duplexes (that is, extended duplexes provided by the composition for detecting the second target nucleic acid, and tag duplexes provided by the composition for detecting the third target nucleic acid) are in associated form, and the third detection temperature may be selected to be a temperature at which all duplexes are in dissociated form. In such a case, the composition for detecting the first target nucleic acid, in the presence of the first target nucleic acid, provides a signal change at the first detection temperature (that is, an extended duplex in associated form is generated upon target nucleic acid amplification, such that the quencher molecule is separated from the reporter molecule, thereby unquenching the signal from the reporter molecule), and provides a constant signal at the second and third detection temperatures (that is, the extended duplex, even when generated, is all in dissociated form such that the quencher molecule is in close proximity to the reporter molecule, thereby quenching the signal from the reporter molecule); the composition for detecting the second target nucleic acid, in the presence of the second target nucleic acid, provides a constant signal at the first and third detection temperatures (that is, the two types of duplexes are both in associated form such that the quencher molecule is in close proximity to the reporter molecule, thereby quenching the signal from the reporter molecule, or the two types of duplexes are both in dissociated form that the quencher molecule separates from the reporter molecule, thereby unquenching the signal from the reporter molecule) and provides a signal change at the second detection temperature (as the duplex between uncleaved PTO and CTO is consumed and an extended duplex in associated form is generated, the quencher molecule is in close proximity to the reporter molecule, thereby quenching the signal from the reporter molecule); and the composition for detecting the third target nucleic acid, in the presence of the third target nucleic acid, provides a constant signal at the first and second detection temperatures (that is, in a tag duplex in associated form (an uncleaved tag duplex and an activated tag duplex fragment) at least one of the two quenchers is in close proximity to the reporter molecule, thereby quenching the signal from the reporter molecule), and provides a signal change at the third detection temperature (that is, an activated tag duplex generated from target nucleic acid amplification is in dissociated form, such that the two quencher molecules both separate from the reporter molecule, thereby unquenching the signal from the reporter molecule).

In a particular embodiment, when n is 3, the composition for detecting a first target nucleic acid may be an UnderSC composition, the composition for detecting a second target nucleic acid may be an InterSC composition, and the composition for detecting a third target nucleic acid may be an InterSC composition.

In a particular embodiment, when n is 3, the composition for detecting a first target nucleic acid may be an InterSC composition, the composition for detecting a second target nucleic acid may be an InterSC composition, and the composition for detecting a third target nucleic acid may be an OverSC composition.

In a particular embodiment, when n is 3, the composition for detecting a first target nucleic acid may be an InterSC composition, the composition for detecting a second target nucleic acid may be an InterSC composition, and the composition for detecting a third target nucleic acid may be an InterSC composition.

In a particular embodiment, when n is 4, the composition for detecting a first target nucleic acid may be an UnderSC or InterSC composition, the composition for detecting a second target nucleic acid and the composition for detecting a third target nucleic acids may be both an InterSC composition, and the composition for detecting a fourth target nucleic acid may be an InterSC or OverSC composition. The method according to the present disclosure exploits the fact that the signal-changing temperature range exists depending on the signal generation mechanism of the composition for detecting a target nucleic acid.

In one embodiment, the detection temperatures according to the present disclosure may be pre-determined in light of the signal-changing temperature range of each of the n compositions for detecting target nucleic acids.

In one embodiment, the signal-changing temperature range of any one of the n compositions for detecting target nucleic acids may be determined depending on the length and/or sequence of the duplex. That is, by adjusting the Tm value of the duplex, the signal-changing temperature range may be predetermined.

In one embodiment, when the signal change is generated by a label-linked oligonucleotide that specifically hybridizes to the target nucleic acid (e.g., a LUX probe, a molecular beacon probe, a HyBeacon probe, an adjacent hybridization probe, etc.), the detection of signals may be successfully achieved at a predetermined detection temperature by adjusting the Tm value of the label-linked oligonucleotide.

In one embodiment, when a Scorpion primer is used as the label-linked oligonucleotide, the detection of signals is successfully carried out at a predetermined temperature, by adjusting the Tm value of a portion that hybridizes to an extended strand.

In one embodiment, when the signal is generated by a duplex formed based on the presence of the target nucleic acid sequence, the detection of signals is successfully carried out at a predetermined temperature by adjusting the Tm value of the duplex. For example, when the signal is generated by a PTOCE method, the detection of signals is successfully achieved at a predetermined temperature by adjusting the Tm value of an extended duplex formed by extension of a PTO fragment on a CTO.

The PTOCE-based method has an advantage that it is easy to control the Tm value of the duplex, or the Tm value of a third hybridization product whose hybridization is influenced by the duplex.

As described above, the detection temperature is determined in view of the signal-changing temperature range that varies depending on the duplex provided by the composition for detecting a target nucleic acid.

In one embodiment, the detection temperature of any one of the n compositions for the detection of n target nucleic acids may be predetermined within a signal-changing temperature range that does not overlap with the signal-changing temperature range of the other compositions (see FIG. 4).

In one embodiment, the detection temperatures assigned to the compositions for detecting the target nucleic acids are different from each other by at least 2° C., 3° C., 4° C., 5° C., 7° C., 8° C., 9° C., 10° C., 11° C., 12° C., 15° C., or 20° C. or more.

In one embodiment, the n detection temperatures may be selected from a temperature range of 45° C. to 97° C., 45° C. to 96° C., 45° C. to 95° C., 45° C. to 94° C., 45° C. to 93° C., 45° C. to 92° C., 45° C. to 91° C., 45° C. to 90° C., 46° C. to 97° C., 46° C. to 96° C., 46° C. to 95° C., 46° C. to 94° C., 46° C. to 93° C., 46° C. to 92° C., 46° C. to 91° C., 46° C. to 90° C., 47° C. to 97° C., 47° C. to 96° C., 47° C. to 95° C., 47° C. to 94° C., 47° C. to 93° C., 47° C. to 92° C., 47° C. to 91° C., 47° C. to 90° C., 48° C. to 97° C., 48° C. to 96° C., 48° C. to 95° C., 48° C. to 94° C., 48° C. to 93° C., 48° C. to 92° C., 48° C. to 91° C., 48° C. to 90° C., 49° C. to 97° C., 49° C. to 96° C., 49° C. to 95° C., 49° C. to 94° C., 49° C. to 93° C., 49° C. to 92° C., 49° C. to 91° C., 49° C. to 90° C., 50° C. to 97° C., 50° C. to 96° C., 50° C. to 95° C., 50° C. to 94° C., 50° C. to 93° C., 50° C. to 92° C., 50° C. to 91° C., or 50° C. to 90° C.

For example, the highest detection temperature (that is, the n^{th} detection temperature) among the above detection temperatures may be selected from a temperature range of 70° C. to 97° C., 70° C. to 95° C., 70° C. to 93° C., 70° C. to 90° C., 73° C. to 97° C., 73° C. to 95° C., 73° C. to 93° C., 73° C. to 90° C., 75° C. to 97° C., 75° C. to 95° C., 75° C. to 93° C., 75° C. to 90° C., 78° C. to 97° C., 78° C. to 95° C., 78° C. to 93° C., 78° C. to 90° C., 80° C. to 97° C., 80° C. to 95° C., 80° C. to 93° C., 80° C. to 90° C., 83° C. to 97° C., 83° C. to 95° C., 83° C. to 93° C., 83° C. to 90° C., 85° C. to 97° C., 85° C. to 95° C., 85° C. to 93° C., or 85° C. to 90° C.

For example, the lowest detection temperature (that is, a first detection temperature) among the n detection temperatures may be selected from a temperature range of 45° C. to 70° C., 45° C. to 68° C., 45° C. to 65° C., 45° C. to 63° C., 45° C. to 60° C., 45° C. to 58° C., 45° C. to 55° C., 48° C. to 70° C., 48° C. to 68° C., 48° C. to 65° C., 48° C. to 63° C., 48° C. to 60° C., 48° C. to 58° C., 48° C. to 55° C., 50° C. to 70° C., 50° C. to 68° C., 50° C. to 65° C., 50° C. to 63° C., 50° C. to 60° C., 50° C. to 58° C., or 50° C. to 55° C.

For example, intermediate detection temperatures (for example, from a second detection temperature to the (n−1)^{th} detection temperature) among the n detection temperatures may be selected from a temperature range of 55° C. to 85° C., 55° C. to 83° C., 55° C. to 80° C., 55° C. to 78° C., 55° C. to 7.5° C., 55° C. to 73° C., 55° C. to 70° C., 55° C. to 68° C., 55° C. to 65° C., 55° C. to 63° C., 55° C. to 60° C., 58° C. to 85° C., 58° C. to 83° C., 58° C. to 80° C., 58° C. to 78° C., 58° C. to 75° C., 58° C. to 73° C., 58° C. to 70° C., 58° C. to 68° C., 58° C. to 65° C., 58° C. to 63° C., 58° C. to 60° C., 60° C. to 85° C., 60° C. to 83° C., 60° C. to 80° C., 60° C. to 78° C., 60° C. to 75° C., 60° C. to 73° C., 60° C. to 70° C., 60° C. to 68° C., 60° C. to 65° C., 60° C. to 63° C., 63° C. to 85° C., 63° C. to 83° C., 63° C. to 80° C., 63° C. to 78° C., 63° C. to 75° C., 63° C. to 73° C., 63° C. to 70° C., 63° C. to 68° C., 63° C. to 65° C., 65° C. to 85° C., 65° C. to 83° C., 65° C. to 80° C., 65° C. to 78° C., 65° C. to 75° C., 65° C. to 73° C., 65° C. to 70° C., 65° C. to 68° C., 68° C. to 85° C., 68° C. to 83° C., 68° C. to 80° C., 68° C. to 78° C., 68° C. to 75° C., 68° C. to 73° C., 68° C. to 70° C., 70° C. to 85° C., 70° C. to 83° C., 70° C. to 80° C., 70° C. to 78° C., 70° C. to 75° C., or 70° C. to 73° C.

According to an embodiment, the n target nucleic acids are respectively assigned to n detection temperatures, n compositions for detecting the n target nucleic acids appropriate for the detection temperatures are prepared, and then step (a) may be performed.

In one embodiment, when n is 3, the first detection temperature may be selected from a temperature range of 50° C. to 60° C., the second detection temperature may be selected from a temperature range of 65° C. to 75° C., and the third detection temperature may be selected from a temperature range of 80° C. to 95° C.

In in step (a), signals are detected at the n detection temperatures during the incubation.

In one embodiment, the detection of signals may be carried out at each cycle or selected cycles, or at an end-point of reaction.

In one embodiment, the detection of signals may be carried out at at least one cycle. For example, the signals may be detected at n detection temperatures in one cycle selected, or may be detected at n detection temperatures in each of two cycles selected. For example, when n is 3 and signals are detected at cycle 1 and cycle 30, the signals (that is, a first signal, a second signal, and a third signal) are detected at a first detection temperature, a second detection temperature, and a third detection temperature at cycle 1, and the signals are detected at a first detection temperature, a second detection temperature, and a third detection temperature at cycle 30.

In one embodiment, the detection of the signals may be carried out at at least two cycles.

In one embodiment, the signal change may be measured using the signals detected at the at least two cycles. For example, the amplification of nucleic acids may be carried out over 30 cycles, 40 cycles, 45 cycles, or 50 cycles of PCR, and at each cycle, signals may be measured at n detection temperatures. Then, the values of signals detected at each detection temperature in a plurality of cycles may be depicted as an amplification curve (a collection of data points of, cycles and RFUs at cycles) at each detection temperature. As a specific example, when n is 3, when each target nucleic acid is present, an amplification curve at the first detection temperature, an amplification curve at the second detection temperature, and an amplification curve at the third detection temperature may be obtained, and change in signal may be identified from the amplification curves.

The term “amplification curve” used herein refers to a curve resulted from a signal-generation reaction, in particular an amplification reaction of a target analyte (in particular, a target nucleic acid). The amplification curve includes a curve resulted from a reaction in the presence of the target nucleic acid in the sample, and a curve or line resulted from a reaction in the absence of the target nucleic acid in the sample.

In one embodiment, a signal change and/or a constant signal may be measured from an indicator indicative of amplification of a target nucleic acid.

As used herein, the term “indicator indicative of amplification” refers to any indicator which is closely related to the occurrence of amplification of a target nucleic acid, obtainable from the signal provided in step (a). The indicator may refer to a value which is generated dependently on the amplification of a target nucleic acid. The indicator may be an indicator that provides a greater value as the amplification of the target nucleic acid increases (that is, as the amount of the target nucleic acid increases), or may be an indicator that provides a smaller value as the amplification increases. The indicator may be any indicator as long as it indicates amplification.

In one embodiment, the indicator indicative of amplification may include one obtained from an amplification curve or a melting curve. In particular, the indicator may include a signal value (e.g., RFU) at a particular cycle, a signal value at each cycle, a difference in signal values between particular cycles, or a difference between a reference signal value and a signal value at a particular cycle in an amplification curve, or height, width or area of the maximum melting peak in a melting curve. In one embodiment, the indicator may be a Ct (cycle threshold) value, ΔRFU (e.g., difference in RFUs at two cycles, difference between a reference RFU and a RFU at a particular cycle, etc.), RFU ratio (e.g., ratio of RFUs at two cycles or ratio of RFUs between a reference RFU and a RFU at a particular cycle, etc.), and a melting peak height/area/width (e.g., height/area/width of the maximum peak in a melting curve), but is not limited thereto.

According to an embodiment, the indicator indicative of amplification is the Ct value. The Ct value may be a crossing point between an amplification curve and a threshold line. The concept of the Ct value is well known in the art.

According to an embodiment, the indicator indicative of amplification is the ΔRFU or RFU ratio between RFU values obtained in an amplification reaction. For example, the indicator is the difference (subtraction) or ratio between RFUs at two cycles, or the difference (subtraction) or ratio between a RFU at a particular cycle and a reference RFU.

According to one embodiment, the indicator indicative of amplification is the melting peak area or width. The melting peak area or width refers to the area or width of the maximum peak in the derivative function of the melting curve obtained for the melting analysis. The melting peak area or width is widely known in the art.

In one embodiment, the detection of signals may be carried out at one cycle. In this case, it is difficult to measure a signal change by a signal value detected at the one cycle alone. Therefore, the signal change may be detected using a separate reference signal value.

The method according to the present disclosure exploits the fact that the composition for detecting a target nucleic acid provides a signal change depending on the presence of the target nucleic acid, only at a corresponding detection temperature. According to one embodiment, the method according to the present disclosure can measure a signal change using signal values detected at detection temperatures at at least two cycles. In another embodiment, the method according to the present disclosure can measure a signal change by using a signal value at a detection temperature, detected in one cycle (that is, a signal value detected in step (a)), and a reference signal value.

In one embodiment, when signals are detected at a plurality of cycles in step (a), the first cycle and the end cycle at which signals are detected, may be selected to be separated from each other by at least 1 cycle to 20 cycles in between. In particular, the first cycle and the end cycle at which the signals are detected may be selected to be separated from each other by 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, 15 cycles, 16 cycles, 17 cycles, 18 cycles, 19 cycles, or 20 cycles or more, and more specifically, by 5 cycles, 10 cycles, 15 cycles, 20 cycles, or 30 cycles or more.

In one embodiment, the detection of signals may be performed at any of intermediate cycles including an exponential phase region, or at any of late cycles including a plateau region. For example, signals may be detected at two cycles, one cycle being any of initial cycles including a baseline region and the other cycle being any of intermediate or late cycles, or signals may be detected at two cycles, one cycle being any of intermediate cycles and the other cycle being any of intermediate or late cycles.

In one embodiment, the initial cycles include cycles up to a cycle adjacent to the value obtained by dividing the end cycle by 3. For example, when the end cycle is 45, 45 divided by 3 results in 15, and thus, the initial cycles may be determined to be from cycle 1 to cycle 20, cycle 1 to cycle 15, cycle 1 to cycle 10, or cycle 1 to cycle 5. The intermediate cycles may be determined to be cycles adjacent to a value obtained by dividing the end cycle by 2. For example, when the end cycle is cycle 45, 45 divided by 2 results in 22.5, and thus, the intermediate cycles may be determined to be from cycle 16 to cycle 30, cycle 18 to cycle 30, cycle 20 to cycle 30, cycle 16 to cycle 27, cycle 18 to cycle 27, cycle 20 to cycle 27, cycle 16 to cycle 25, cycle 18 to cycle 25, or cycle 20 to cycle 25. The late cycles may be determined to be the end cycle of an amplification reaction, or cycles adjacent to the end cycle. For example, when the end cycle is cycle 45, the late cycles may be determined to be from cycle 31 to cycle 45, cycle 35 to cycle 45, cycle 38 to cycle 45, cycle 40 to cycle 45, or cycle 43 to cycle 45. The initial cycles, intermediate cycles and late cycles may be changed depending on the final number of cycles of the amplification reaction. In particular, in step (a), the cycle at which the signal is detected may be any of the late cycles (e.g., the end cycle), and the cycle at which the reference signal may be any of the initial cycles (e.g., cycle 1) of the positive control reaction.

In one embodiment, the signal change may be measured using a signal detected at the at least one cycle and a “reference signal value”. The reference signal value may refer to a value that can confirm a signal change dependent on the presence of the target nucleic acid through a separate reaction.

In one embodiment, the reference signal value may be obtained from a reaction in the absence of a corresponding target nucleic acid at a corresponding detection temperature. For example, the reference signal value may be a “signal value at a detection temperature” in the absence of the target nucleic acid.

In one embodiment, there are n reference signal values for n detection temperatures.

In one embodiment, the “signal value at a detection temperature (e.g., an i^{th} detection temperature)” detected in the absence of the target nucleic acid (e.g., an i^{th} target nucleic acid) may be obtained through a separate negative control reaction.

In one embodiment, the reference signal value may be obtained by performing a negative control reaction at the same time as or separately from the method according to the present disclosure.

In one embodiment, the reference signal value may be obtained through a negative control reaction. According to one particular embodiment, the reference signal value at the i^{th} detection temperature may be obtained by mixing a sample free of the i^{th} target nucleic acid (e.g., distilled water) with n compositions for detecting target nucleic acids and detecting signals at the i^{th} detection temperature while amplifying nucleic acids. Here, the detection of signals may be performed at any cycle. In particular, a signal value detected at any of the initial cycles of the negative control reaction may be used as the reference signal value, or a signal value detected at any of the late cycles of the negative control reaction may be used as the reference signal value. More specifically, a signal value detected in the same cycle as the cycle at which the signal is detected in step (a) may be used as the reference signal value.

In one embodiment, the reference signal value may be obtained through a positive control reaction. According to a particular embodiment, the reference signal value may be obtained by mixing a sample containing an i^{th} target nucleic acid with the composition for detecting an i^{th} target nucleic acid, and detecting a signal at an i^{th} detection temperature while amplifying nucleic acids.

When the reference signal value is obtained through the positive control reaction, the cycle at which a signal value is detected may be a cycle in a baseline region of the positive control reaction. The baseline region refers to a region in which a signal (e.g., a fluorescent signal) remains substantially constant during the initial cycles of an amplification reaction (e.g., PCR). In this region, the level of amplification products is not enough to be detectable, most of the fluorescent signals in this region are attributed to the fluorescent signal inherent to the reaction sample, and the background signal including the fluorescent signals of the measurement system itself. That is, a signal value detected at a cycle in the baseline region of the positive control reaction is substantially identical to the reference signal value obtained from a reaction in the absence of the target nucleic acid (e.g., the negative control reaction).

In one embodiment, a signal change may be measured through a difference between the reference signal value and the signal value detected in step (a).

In one embodiment, the reference signal value may be a threshold value predetermined from a negative control reaction, by taking into consideration the background signal and sensitivity of the detector, or characteristics of the labels used. Using the threshold value, significance of a signal change may be determined. The threshold value may be determined by a known threshold setting method. For example, the threshold value may be determined in view of the background signal, sensitivity, label characteristics, signal variation of a detector, or margin of errors, and the like.

In one embodiment, when a threshold value is used as the reference signal value, if the signal value detected in step (a) is equal to or greater than the threshold value, it may be determined that the signal has changed.

In one embodiment, detection of signals at each of the n detection temperatures may be performed using a single type of detector.

In one embodiment, the single type of detector is one detector. In one embodiment, the signals from the labels in each of the label-linked oligonucleotides included in the n compositions for detecting target nucleic acids are not differentiated from each other by a single type of detector with respect to each target nucleic acid.

As used herein, a single or one type of fluorescent label refers to a fluorescent label that has identical or substantially identical signal characteristics (e.g., optical characteristics, emission wavelength, and electric signals).

For example, FAM and CAL Fluor 610 provide different types of signals. In the present application, the single or one type of fluorescent label means that signals from the fluorescent label are not differentiated from each other, using a detection channel. Such a single or one type of fluorescent label does not depend on the chemical structure of the fluorescent label, and as such, even when two fluorescent labels have different chemical structures from each other, if they are not differentiated using a detection channel, they are considered as one type.

According to the present disclosure, signals generated from the n compositions for detecting target nucleic acids that include one type of fluorescent label in common are not differentiated by one detection channel.

The term “detection channel” as used herein refers to a means for detecting a signal from a single type of a fluorescent label. Thermocyclers usable in the art, e.g., ABI 7500 (Applied Biosystems), QuantStudio (Applied Biosystems), CFX96 (Bio-Rad Laboratories), Cobas z 480 (Roche), LightCycler (Roche), etc. include a several channels (e.g., optical diodes) for detecting signals from a few different types of fluorescent labels, and these channels correspond to the detection channel used herein.

The detection channel used in the present invention includes a means for detecting signals. For example, the detection channel may be an optical diode capable of detecting a fluorescent signal at a particular wavelength.

According to an embodiment, the signals detected at the n detection temperatures are not differentiated from each other by the single type of detector.

### Step (b): Determining the Presence of a Target Nucleic Acid

After detection of signals, the presence of n target nucleic acids is determined from the signals detected in step (a).

In one embodiment, the presence of the i^{th} target nucleic acid is determined by the signal change detected at the i^{th} detection temperature. For example, a signal change is measured from the signals detected at the i^{th} detection temperature, to determine the presence of the i^{th} target nucleic acid.

In one embodiment, when a change in signal at the i^{th} detection temperature is measured, it may be determined that the i^{th} target nucleic acid is present.

In one embodiment, if the signal is constant at the i^{th} detection temperature, it may be determined that the i^{th} target nucleic acid is absent.

In one embodiment, the signal change can be measured using signals detected at at least two cycles, or a signal detected at at least one cycle and a “reference signal value”.

Determining the presence of the target nucleic acid from signals detected at each detection temperature may be carried out by the process described in step (a) for measuring a signal change, e.g., a method using a label indicative of amplification, and other various methods known in the art.

In a particular embodiment, when n is 3 and detection of signals is carried out at cycle 10, cycle 20, and cycle 30, the presence of the first target nucleic acid may be determined from signals detected at a first detection temperature (a first signal at cycle 10, a first signal at cycle 20, and a first signal at cycle 30), the presence of the second target nucleic acid may be determined from signals detected at a second detection temperature (a second signal at cycle 10, a second signal at cycle 20, and a second signal at cycle 30), and the presence of the third target nucleic acid may be determined from signals detected at a third detection temperature (a third signal at cycle 10, a third signal at cycle 20, and a third signal at cycle 30).

In a particular embodiment, when n is 4 and detection of signals is performed at cycle 30, the presence of the first target nucleic acid is determined from a signal detected at a first detection temperature (that is, a first signal at cycle 30) and a reference signal value, the presence of the second target nucleic acid is determined from a signal detected at a second detection temperature (that is, a second signal at cycle 30) and a reference signal value, and the presence of the third target nucleic acid is determined from a signal detected at a third detection temperature (that is, a third signal at cycle 30) and a reference signal value.

In one embodiment, the reference signal value may be obtained through a separate negative control reaction or positive control reaction.

In one embodiment, the method according to the present disclosure may be performed along with a negative control reaction. A signal value detected in the negative control reaction may be used as a reference signal value. For example, a signal detected at one cycle (e.g., the end cycle) at an i^{th} detection temperature in a reaction comprising the composition for detecting an i^{th} target nucleic acid, may be compared with a signal detected at the same cycle (e.g., the end cycle) at the same detection temperature (that is, the i^{th} detection temperature) in the negative control reaction to determine whether or not the signal has changed.

In a particular embodiment, when n is 3 and a signal is detected at cycle 30, the presence of the first target nucleic acid may be determined from a signal detected at the first detection temperature (that is, a first signal at cycle 30) and a first reference signal value (e.g., a signal at the first detection temperature detected at cycle 30 of a negative control reaction), the presence of the second target nucleic acid may be determined from a signal detected at the second detection temperature (that is, a second signal at cycle 30) and a second reference signal value (e.g., a signal at the second detection temperature detected at cycle 30 of a negative control reaction), and the presence of the third target nucleic acid may be determined from a signal detected at the third detection temperature (that is, a third signal at cycle 30) and a third reference signal value (e.g., a signal at the third detection temperature detected at cycle 30 of a negative control reaction).

In one embodiment, the method according to the present disclosure may be performed along with a positive control reaction. A signal value detected in the positive control reaction may be used as a reference signal value. For example, a signal detected at one cycle at an i^{th} detection temperature, for example, at cycle 30, a first signal detected at cycle 30 may be compared with a signal detected at the i^{th} detection temperature in a positive control reaction at a cycle prior to cycle 30, e.g., cycle 1, to determine whether the signal has changed.

In one embodiment, when signal detection is performed at one cycle in step (a) and a signal change is measured using a reference signal value obtained through a positive control reaction, the signal detection in the positive control reaction to obtain the reference signal value may be performed at a cycle that is at least 30 cycles, 20 cycles, 10 cycles, or 5 cycles prior to the cycle at which the signal detection is performed in step (a).

In a particular embodiment, when n is 3 and signal detection is performed at cycle 30, the presence of the first target nucleic acid may be determined from a signal detected at the first detection temperature (that is, a first signal at cycle 30) and a first reference signal value (e.g., a signal at the first detection temperature detected at cycle 1 of a positive control reaction of the first target nucleic acid), the presence of the second target nucleic acid may be determined from a signal detected at the second detection temperature (that is, a second signal at cycle 30) and a second reference signal value (e.g., a signal at the second detection temperature detected at cycle 1 of a positive control reaction of the second target nucleic acid), and the presence of the third target nucleic acid may be determined from a signal detected at the third detection temperature (that is, a third signal at cycle 30) and a third reference signal value (e.g., a signal at the third detection temperature detected at cycle 1 of a positive control reaction of the third target nucleic acid).

According to another aspect of the present disclosure, provided is a method for detecting two target nucleic acids in a sample, comprising:
(a) detecting signals at a first detection temperature and a second detection temperature, while incubating the sample suspected of containing at least one of the two target nucleic acids with a composition for detecting a first target nucleic acid and a composition for detecting a second target nucleic acid in a reaction vessel; wherein the incubation comprises a plurality of cycles, and the detection of signals is carried out at at least one of the plurality of cycles, wherein the composition for detecting the first target nucleic acid provides a signal change at the first detection temperature and provides a constant signal at the second detection temperature in the presence of the first target nucleic acid, the signal change indicating the presence of the first target nucleic acid; and the composition for detecting the second target nucleic acid provides a signal change at the second detection and provides a constant signal at the first detection temperature in the presence of the second target nucleic acid, the signal change indicating the presence of the second target nucleic acid, and wherein the first detection temperature is lower than the second detection temperature, and (b) determining the presence of the two target nucleic acids from the signals detected in step (a); wherein the presence of the first target nucleic acid is determined by the signal change detected at the first detection temperature, and the presence of the second target nucleic acid is determined by the signal change detected at the second detection temperature.

According to another aspect of the present disclosure, provided is a method for detecting three target nucleic acids in a sample, comprising:
(a) detecting signals at a first detection temperature, a second detection temperature, and a third detection temperature, while incubating the sample suspected of containing at least one of the three target nucleic acids with a composition for detecting a first target nucleic acid, a composition for detecting a second target nucleic acid, and a composition for detecting a third target nucleic acid in a reaction vessel; wherein the incubation comprises a plurality of cycles, and the detection of signals is carried out at at least one of the plurality of cycles, wherein the composition for detecting the first target nucleic acid provides a signal change at the first detection temperature and provides a constant signal at the second detection temperature and the third detection temperature in the presence of the first target nucleic acid, the signal change indicating the presence of the first target nucleic acid; the composition for detecting the second target nucleic acid provides a signal change at the second detection temperature and provides a constant signal at the first detection temperature and the third detection temperature in the presence of the second target nucleic acid, the signal change indicating the presence of the second target nucleic acid; and the composition for detecting the third target nucleic acid provides a signal change at the third detection temperature and provides a constant signal at the first detection temperature and the second detection temperature in the presence of the third target nucleic acid, the signal change indicating the presence of the third target nucleic acid, and wherein the first detection temperature is lower than the second detection temperature, and the second detection temperature is lower than the third detection temperature, and (b) determining the presence of the three target nucleic acids from the signals detected in step (a), wherein the presence of the first target nucleic acid is determined by the signal change detected at the first detection temperature, the presence of the second target nucleic acid is determined by the signal change detected at the second detection temperature, and the presence of the third target nucleic acid is determined by the signal change detected at the third detection temperature.

Since the second embodiment and third embodiment of the present disclosure follow the same principles as the first embodiment of the present disclosure described above, the features common between these embodiments will not be redundantly described in the interest of clarity of the present application.

### II. Kit for Detecting Target Nucleic Acids

According to another aspect of the present disclosure, provided is a kit, comprising n compositions for detecting n target nucleic acids in a sample, wherein n is an integer of 2 or more, wherein each of the n compositions for detecting the n target nucleic acids provides a signal change at a corresponding detection temperature among n detection temperatures, the signal change indicating the presence of a corresponding target nucleic acid, wherein a composition for detecting an i^{th} target nucleic acid among the n target nucleic acids provides a signal change at an i^{th} detection temperature among the n detection temperatures and provides a constant signal at the other detection temperatures in the presence of the i^{th} target nucleic acid, and wherein i represents an integer from 1 to n, and the i^{th} detection temperature is lower than the (i+1)^{th} detection temperature.

Since the kit of the present disclosure is prepared in order to enable the methods of the present disclosure, the common features shared between the two will not be redundantly described for clarity of the present specification.

According to an embodiment, in a temperature range covering all of the n detection temperatures, the composition for detecting the i^{th} target nucleic acid has a “signal-changing temperature range” in which the signal changes depending on the presence of the i^{th} target nucleic acid, and a “signal-constant temperature range” in which the signal is constant even in the presence of the i^{th} target nucleic acid.

According to an embodiment, the composition for detecting the i^{th} target nucleic acid has one or two signal-constant temperature ranges.

According to an embodiment, the composition for detecting the i^{th} target nucleic acid is any one of: (i) an Under-Signal-Change (UnderSC) composition having a characteristic that the signal-changing temperature range is lower than the signal-constant temperature range; (ii) an Over-Signal-Change (OverSC) composition having a characteristic that the signal-changing temperature range is higher than the signal-constant temperature range; and (iii) an Inter-Signal-Change (InterSC) composition having a characteristic that the signal-changing temperature range is higher than one of two signal-constant temperature ranges, and lower than the other of the two signal-constant temperature ranges.

According to an embodiment, the i^{th} detection temperature is selected from within the signal-changing temperature range of the composition for detecting the i^{th} target nucleic acid, and the i^{th} detection temperature is not included in the signal-changing temperature ranges of the compositions for detecting the other target nucleic acids.

According to an embodiment, the signal-changing temperature range of the composition for detecting the i^{th} target nucleic acid may partially overlap with the signal-changing temperature range of a composition for detecting a target nucleic acid having an adjacent detection temperature, and does not overlap with the signal-changing temperature range of a composition for detecting a target nucleic acid having a detection temperature that is not adjacent thereto.

According to an embodiment, when n is 2, a composition for detecting a first target nucleic acid is an UnderSC composition or an InterSC composition, and a composition for detecting a second target nucleic acid is the InterSC composition or an OverSC composition.

According to an embodiment, when n is 3 or more, the composition for detecting a first target nucleic acid is an UnderSC composition or an InterSC composition, a composition for detecting an n^{th} target nucleic acid is an InterSC composition or an OverSC composition, and the compositions for detecting target nucleic acids other than the first target nucleic acid and the n^{th} target nucleic acid, are an InterSC composition.

According to an embodiment, the composition for detecting the i^{th} target nucleic acid includes a label providing a signal depending on the presence of the i^{th} target nucleic acid.

According to an embodiment, the label is linked to an oligonucleotide, or is incorporated into an oligonucleotide during an incubation of the kit with the sample.

According to an embodiment, the composition for detecting the i^{th} target nucleic acid provides a duplex providing a signal change.

According to an embodiment, the composition for detecting the i^{th} target nucleic acid provides a duplex providing a signal change, and when the duplex exists as a duplex in associated form or in dissociated form, the composition for detecting the i^{th} target nucleic acid provides a signal from the label.

According to an embodiment, signals provided by the n compositions for detecting the n target nucleic acids are not distinguished from each other by a single type of detector.

In one embodiment, signals indicating the presence of each target nucleic acid provided by the n compositions for detecting target nucleic acids are not distinguished from each other by the single type of detector. For example, all of the labels included in the n composition for detecting target nucleic acids are the identical type of label (e.g., one fluorescent label).

In one embodiment, the composition for detecting the i^{th} target nucleic acid provides a duplex providing a signal change, and the signal-changing temperature range of the composition for detecting the i^{th} target nucleic acid is determined depending on the length and/or sequence of the duplex.

According to an embodiment, the kit further comprises an instruction that describes the present method.

According to an embodiment, the instructions for describing or practicing the methods of the present invention may be recorded on a suitable storage medium. For example, the instructions may be printed on a substrate, such as paper and plastic. In other embodiments, the instructions may be present as an electronic storage data file present on a suitable computer readable storage medium such as CD-ROM and diskette. In yet other embodiments, the actual instructions may not be present in the kit, but means for obtaining the instructions from a remote source, e.g., via the internet, are provided. An example of this embodiment is a kit that includes a web address where the instructions can be viewed and/or from which the instructions can be downloaded.

All of the kits described hereinabove may optionally include the reagents required for performing target nucleic acid amplification reactions (e.g., PCR reactions) such as buffers, DNA polymerase cofactors, and deoxyribonucleotide-5-triphosphates. Optionally, the kits may also include various polynucleotide molecules, reverse transcriptase, various buffers and reagents, and antibodies that inhibit DNA polymerase activity. The kits may also include reagents necessary for performing positive and negative control reactions. Optimal amounts of reagents to be used in a given reaction can be readily determined by one of ordinary skill in the art having the benefit of the present disclosure. The above-described constituents of the kits may be present in separate containers, or a plurality of the constituents may be present in a single container.

Hereinafter, the present invention will be described in more detail through embodiments. The following embodiments are provided to describe the present invention in further details, and it will become apparent to one of ordinary skill in the art in the technical field to which this invention belongs that the scope of the present invention as suggested in the appended claims is not limited by the following embodiments.

### EXAMPLES

The present inventors have confirmed whether multiple target nucleic acids can be detected in real time using the same type, that is, a single type of label according to the method of the present disclosure, by using various combinations of an UnderSC composition for detecting a target nucleic acid, an InterSC composition for detecting a target nucleic acid, and an OverSC composition for detecting a target nucleic acid.

In particular, among various signal generation mechanisms adoptable by the three types of compositions for detecting target nucleic acids, the PTOCE-based method, as in FIG. 5, using a CTO to which all of interactive dual labels are linked (hereinbelow, referred to as PTOCE-based-1) was used for the UnderSC composition; the PTOCE-based method as in FIG. 6, in which one of interactive dual labels is linked to a 5′-tagging portion of PTO and the other is linked to a capturing portion of CTO (hereinafter referred to as PTOCE-based-2) was used for the InterSC composition; and the dual quenching method as shown in FIG. 7 was used for the OverSC composition.

For the templates of multiple target nucleic acids, the genomic DNA of Chlamydia trachomatis (CT) (Accession number: ATCC VR-1500, Koram Deo Lab), Neisseria gonorrhoeae (NG) (Accession number: ATCC 700825, Koram Deo Lab) and Ureaplasma parvum (UP) (Accession number: ATCC 27815, Koram Deo Lab) were used. As shown in Table 1 below, the above three target nucleic acids were detected by setting various combinations of the above 3 types of compositions and detection temperatures. Combinations 1 to 4 are the cases where the number of target nucleic acids (n) is 2, and Combination 5 is the case where the number of target nucleic acids (n) is 3.

TABLE 1
Combinations of the compositions for detecting target nucleic acids

| Number of | Com- | Target | Composi- | | Detection |
|---|---|---|---|---|---|
| targets | bina- | nucleic | tion | Signal generation | temperature |
| (n) | tion | acid | type | mechanism | (° C.) |
| 2 | 1 | CT | UnderSC | PTOCE-based-1 | 50 |
| | | NG | InterSC | PTOCE-based-2 | 72 |
| | 2 | CT | UnderSC | PTOCE-based-1 | 57 |
| | | UP | OverSC | Dual quenching | 85 |
| | 3 | CT | InterSC | PTOCE-based-2 | 57 |
| | | NG | InterSC | PTOCE-based-2 | 72 |
| | 4 | NG | InterSC | PTOCE-based-2 | 72 |
| | | UP | OverSC | Dual quenching | 95 |
| 3 | 5 | CT | UnderSC | PTOCE-based-1 | 50 |
| | | NG | InterSC | PTOCE-based-2 | 72 |
| | | UP | OverSC | Dual quenching | 95 |

### Example 1: Preparation of Oligonucleotides for Composition for Detecting Target Nucleic Acids

### <Combination 1>

A first detection temperature for detecting a first signal change indicative of the presence of a first target nucleic acid, CT, was set to 50° C., and a second detection temperature for detecting a second signal change indicative of the presence of a second target nucleic acid, NG, was set to 72° C. Then, the oligonucleotides for the composition for detecting CT target nucleic acid, and the composition for detecting NG target nucleic acid, were prepared as shown in Table 2 below.

The composition for detecting CT target nucleic acid includes a primer pair, a PTO, and a CTO linked with a reporter molecule (CAL Fluor Red 610) and a quencher molecule (BHQ-2), wherein the sequences of the PTO and the CTO were designed such that a duplex between the PTO (that is, uncleaved PTO) and the CTO are in dissociated form at both the first and second detection temperatures, and an extended duplex formed depending on the presence of CT is in associated form at the first detection temperature, and in dissociated form at the second detection temperature (see FIG. 8).

The composition for detecting NG target nucleic acid includes a primer pair, a PTO having a quencher molecule (BHQ-2) linked to its tagging portion, and a CTO having a reporter molecule (CAL Fluor Red 610) linked to its capturing portion, wherein the sequences of the PTO and the CTO were designed such that a duplex between the PTO (that is, uncleaved PTO) and the CTO is in associated form at the first detection temperature, and in dissociated form at the second detection temperature, and an extended duplex formed depending on the presence of NG is in associated form at both the first and second detection temperatures (see FIG. 8).

### <Combination 2>

A first detection temperature for detecting a first signal change indicative of the presence of a first target nucleic acid, CT, was set to 57° C., and a second detection temperature for detecting a second signal change indicative of the presence of a second target nucleic acid, UP, was set to 85° C. Then, oligonucleotides for the composition for detecting CT target nucleic acid and the composition for detecting UP target nucleic acid, were prepared as shown in Table 2 below.

The composition for detecting CT target nucleic acid includes a primer pair, a PTO, and a CTO linked with a reporter molecule (CAL Fluor Red 610) and a quencher molecule (BHQ-2), wherein the sequences of the PTO and the CTO were designed such that a duplex between the PTO (that is, uncleaved PTO) and the CTO is in dissociated form at both the first and second detection temperatures, and an extended duplex formed depending on the presence of CT is in associated form at the first detection temperature, and in dissociated form at the second detection temperature (see FIG. 9).

The composition for detecting UP target nucleic acid includes a primer pair, a PTO linked with a reporter molecule (CAL Fluor Red 610) and a first quencher molecule (BHQ-2), and a CQO linked with a second quencher molecule (BHQ-2), wherein the sequences of the PTO and the CQO were designed such that a tag duplex between the PTO (that is, uncleaved PTO) and the CQO is in associated form at the first detection temperature, and in dissociated form at the second detection temperature. That is, the sequences of the PTO and the CQO were designed such that the tag duplex between the PTO and the CQO is cleaved depending on the presence of the UP target nucleic acid to generate an activated tag duplex fragment, and the activated tag duplex fragment is in associated form at the first detection temperature, and in dissociated form at the second detection temperature (see FIG. 9).

### <Combination 3>

A first detection temperature for detecting a first signal change indicative of the presence of a first target nucleic acid, CT, was to 57° C., and a second detection temperature for detecting a second signal change indicative of the presence of a second target nucleic acid, NG, was set to 72° C. Then, oligonucleotides for the composition for detecting CT target nucleic acid and the composition for detecting NG target nucleic acid, were prepared as shown in Table 2 below.

The composition for detecting CT target nucleic acid includes a primer pair, a PTO having a quencher molecule (BHQ-2) linked to its tagging portion, and a CTO having a reporter molecule (CAL Fluor Red 610) linked to its capturing portion, wherein the sequences of the PTO and the CTO were designed such that a duplex between the PTO (that is, uncleaved PTO) and the CTO is in dissociated form at the first and second detection temperatures, and an extended duplex formed depending on the presence of CT is in associated form at the first detection temperature, and in dissociated form at the second detection temperature (see FIG. 10).

The composition for detecting NG target nucleic acid includes a primer pair, a PTO having a quencher molecule (BHQ-2) linked to its tagging portion, and a CTO having a reporter molecule (CAL Fluor Red 610) linked to its capturing portion, wherein the sequences of the PTO and the CTO were designed such that a duplex between the PTO (that is, uncleaved PTO) and the CTO is in associated form at the first detection temperature, and in dissociated form at the second detection temperature, and an extended duplex formed depending on the presence of NG is in associated form at both the first and second detection temperatures (see FIG. 10).

### <Combination 4>

A first detection temperature for detecting a first signal change indicative of the presence of a first target nucleic acid, NG, was set to 72° C., and a second detection temperature for detecting a second signal change indicative of the presence of a second target nucleic acid, UP, was set to 95° C. Then, oligonucleotides for the composition for detecting NG target nucleic acid and the composition for detecting UP target nucleic acid, were prepared as shown in Table 2 below.

The composition for detecting NG target nucleic acid includes a primer pair, a PTO having a quencher molecule (BHQ-2) linked to its tagging portion, and a CTO having a reporter molecule (CAL Fluor Red 610) linked to its capturing portion, wherein the sequences of the PTO and the CTO were designed such that a duplex between the PTO (that is, uncleaved PTO) and the CTO is in dissociated form at the first and second detection temperatures, and an extended duplex formed depending on the presence of NG is in associated form at the first detection temperature, and in dissociated form at the second detection temperature (see FIG. 11).

The composition for detecting UP target nucleic acid includes a primer pair, a PTO linked with a reporter molecule (CAL Fluor Red 610) and a first quencher molecule (BHQ-2), and a CQO linked with a second quencher molecule (BHQ-2), wherein the sequences of the PTO and the CQO were designed such that a tag duplex between the PTO (that is, uncleaved PTO) and the CQO is in associated form at the first detection temperature, and in dissociated form at the second detection temperature. That is, the sequences of the PTO and the CQO were designed such that the tag duplex between the PTO and the CQO is cleaved depending on the presence of the UP target nucleic acid to generate an activated tag duplex fragment, and the activated tag duplex fragment is in associated form at the first detection temperature, and in dissociated form at the second detection temperature (see FIG. 11).

### <Combination 5>

A first detection temperature for detecting a first signal change indicative of the presence of a first target nucleic acid, CT, was set to 50° C., a second detection temperature for detecting a second signal change indicative of the presence of a second target nucleic acid, NG, was set to 72° C., and a third detection temperature for detecting a third signal change indicative of the presence of a third target nucleic acid, UP, was set to 95° C. Then, oligonucleotides for the composition for detecting CT target nucleic acid, the composition for detecting NG target nucleic acid, and the composition for detecting UP target nucleic acid, were prepared as shown in Table 2 below.

The composition for detecting CT target nucleic acid includes a primer pair, a PTO, and a CTO linked with a reporter molecule (CAL Fluor Red 610) and a quencher molecule (BHQ-2), wherein the sequences of the PTO and the CTO were designed such that a duplex between the PTO (that is, uncleaved PTO) and the CTO is in dissociated form at all of the first to third detection temperatures, and an extended duplex formed depending on the presence of CT is in associated form at the first detection temperature, and in dissociated form at the second and third detection temperatures (see FIG. 12).

The composition for detecting NG target nucleic acid includes a primer pair, a PTO having a quencher molecule (BHQ-2) linked to its tagging portion, and a CTO having a reporter molecule (CAL Fluor Red 610) linked to its capturing portion, wherein the sequences of the PTO and the CTO were designed such that a duplex between the PTO (that is, uncleaved PTO) and the CTO is in associated form at the first detection temperature, and in dissociated form at the second and third detection temperatures, and an extended duplex formed depending on the presence of NG is in associated form at the first and second detection temperatures, and in dissociated form at the third detection temperature (see FIG. 12).

The composition for detecting UP target nucleic acid includes a primer pair, a PTO linked with a reporter molecule (CAL Fluor Red 610) and a first quencher molecule (BHQ-2), and a CQO linked with a second quencher molecule (BHQ-2), wherein the sequences of the PTO and the CQO were designed such that a tag duplex between the PTO (that is, uncleaved PTO) and the CQO is in associated form at the first and second detection temperatures, and in dissociated form at the third detection temperature. That is, the sequences of the PTO and the CQO were designed such that the tag duplex between the PTO and the CQO is cleaved depending on the presence of the UP target nucleic acid to generate an activated tag duplex fragment, and the activated tag duplex fragment is in associated form at the first and second detection temperatures, and in dissociated form at the third detection temperature (see FIG. 12).

The 3′-end of the PTO and the 3′-end of the CTO in PTOCE-based-1 were blocked by Spacer C3 to prohibit extension reactions by DNA polymerase.

TABLE 2
Oligonucleotide sequence

| | Target | SEQ | | |
|---|---|---|---|---|
| Combi- | nucleic | ID | | |
| nation | acid | NO. | Oligo type | Sequence (5′-3′) |
| 1 | CT | 1 | Forward | GAGTTTTAAAATGGGAAATTCTGGT |
| | | | primer | |
| 1 | CT | 2 | Reverse | CCAATTGTAATAGAAGCATTGGTTG |
| | | | primer | |
| 1 | CT | 3 | PTO | GATTACGCGACCGCATCAGAAGCTGTCATTTT |
| | | | | GGCTGCG[SpacerC3] |
| 1 | CT | 4 | CTO | [BHQ-2]TATTTTATTTTTATTTT[T(CAL Fluor Red |
| | | | | 610)]TTTATGTGCGGTCGCGTAATC[Spacer C3] |
| 1 | NG | 5 | Forward | CAATGGATCGGTATCACTCGC |
| | | | primer | |
| 1 | NG | 6 | Reverse | TACGCCTGCTACTTTCACGC |
| | | | primer | |
| 1 | NG | 7 | PTO | [BHQ-2] |
| | | | | GTACGCGATACCGGGCCCCTCATTGGCGTGT |
| | | | | TTCG[SpacerC3] |
| 1 | NG | 8 | CTO | GGCTCGACTGTCGTCTCTGCTCATGCCCGGTAT |
| | | | | CGCGTAC[Cal Fluor Red 610] |
| 2 | CT | 1 | Forward | GAGTTTTAAAATGGGAAATTCTGGT |
| | | | primer | |
| 2 | CT | 2 | Reverse | CCAATTGTAATAGAAGCATTGGTTG |
| | | | primer | |
| 2 | CT | 3 | PTO | GATTACGCGACCGCATCAGAAGCTGTCATTTT |
| | | | | GGCTGCG[SpacerC3] |
| 2 | CT | 4 | CTO | [BHQ-2]TATTTTATTTTTATTTT[T(CAL Fluor Red |
| | | | | 610)]TTTATGTGCGGTCGCGTAATC[Spacer C3] |
| 2 | UP | 9 | Forward | CAGATTTAIACGGTATTACAACIGGTG |
| | | | primer | |
| 2 | UP | 10 | Reverse | TTACTTCAGCAITACCTAATTTATCATCIAAC |
| | | | primer | |
| 2 | UP | 11 | PTO | [BHQ-2] |
| | | | | CACGTAGGGTTTTTCCACCACCAT[T(CALFluor |
| | | | | Red610)]CTCGCATCCGGCCAGCTCCTCTCCAG |
| | | | | G[SpacerC3] |
| 2 | UP | 12 | CQO | CCTGGAGAGGAGCTGGCCGGATGCGAGA[BHQ-2] |
| 3 | CT | 1 | Forward | GAGTTTTAAAATGGGAAATTCTGGT |
| | | | primer | |
| 3 | CT | 2 | Reverse | CCAATTGTAATAGAAGCATTGGTTG |
| | | | primer | |
| 3 | CT | 13 | PTO | [BHQ-2] |
| | | | | CATGGCCAAGGCATCAGAAGCTGTCATTTTG |
| | | | | GCTGCG[SpacerC3] |
| 3 | CT | 14 | CTO | TTTTTTTTATTTTTTTTTATGCCTTGGCCATG[Cal |
| | | | | Fluor Red 610] |
| 3 | NG | 5 | Forward | CAATGGATCGGTATCACTCGC |
| | | | primer | |
| 3 | NG | 6 | Reverse | TACGCCTGCTACTTTCACGC |
| | | | primer | |
| 3 | NG | 7 | PTO | [BHQ-2] |
| | | | | GTACGCGATACCGGGCCCCTCATTGGCGTGT |
| | | | | TTCG[SpacerC3] |
| 3 | NG | 8 | CTO | GGCTCGACTGTCGTCTCTGCTCATGCCCGGTAT |
| | | | | CGCGTAC[Cal Fluor Red 610] |
| 4 | NG | 5 | Forward | CAATGGATCGGTATCACTCGC |
| | | | primer | |
| 4 | NG | 6 | Reverse | TACGCCTGCTACTTTCACGC |
| | | | primer | |
| 4 | NG | 7 | PTO | [BHQ-2] |
| | | | | GTACGCGATACCGGGCCCCTCATTGGCGTGT |
| | | | | TTCG[SpacerC3] |
| 4 | NG | 8 | CTO | GGCTCGACTGTCGTCTCTGCTCATGCCCGGTAT |
| | | | | CGCGTAC[Cal Fluor Red 610] |
| 4 | UP | 9 | Forward | CAGATTTAIACGGTATTACAACIGGTG |
| | | | primer | |
| 4 | UP | 10 | Reverse | TTACTTCAGCAITACCTAATTTATCATCIAAC |
| | | | primer | |
| 4 | UP | 11 | PTO | [BHQ-2] |
| | | | | CACGTAGGGTTTTTCCACCACCAT[T(CALFluor |
| | | | | Red610)]CTCGCATCCGGCCAGCTCCTCTCCAG |
| | | | | G[SpacerC3] |
| 4 | UP | 12 | CQO | CCTGGAGAGGAGCTGGCCGGATGCGAGA[BHQ-2] |
| 5 | CT | 1 | Forward | GAGTTTTAAAATGGGAAATTCTGGT |
| | | | primer | |
| 5 | CT | 2 | Reverse | CCAATTGTAATAGAAGCATTGGTTG |
| | | | primer | |
| 5 | CT | 3 | PTO | GATTACGCGACCGCATCAGAAGCTGTCATTTT |
| | | | | GGCTGCG[SpacerC3] |
| 5 | CT | 4 | CTO | [BHQ-2]TATTTTATTTTTATTTT[T(CAL Fluor Red |
| | | | | 610)]TTTATGTGCGGTCGCGTAATC[Spacer C3] |
| 5 | NG | 5 | Forward | CAATGGATCGGTATCACTCGC |
| | | | primer | |
| 5 | NG | 6 | Reverse | TACGCCTGCTACTTTCACGC |
| | | | primer | |
| 5 | NG | 7 | PTO | [BHQ-2] |
| | | | | GTACGCGATACCGGGCCCCTCATTGGCGTGT |
| | | | | TTCG[SpacerC3] |
| 5 | NG | 8 | CTO | GGCTCGACTGTCGTCTCTGCTCATGCCCGGTAT |
| | | | | CGCGTAC[Cal Fluor Red 610] |
| 5 | UP | 9 | Forward | CAGATTTAIACGGTATTACAACIGGTG |
| | | | primer | |
| 5 | UP | 10 | Reverse | TTACTTCAGCAITACCTAATTTATCATCIAAC |
| | | | primer | |
| 5 | UP | 11 | PTO | [BHQ-2] |
| | | | | CACGTAGGGTTTTTCCACCACCAT[T(CALFluor |
| | | | | Red610)]CTCGCATCCGGCCAGCTCCTCTCCAG |
| | | | | G[SpacerC3] |
| 5 | UP | 12 | CQO | CCTGGAGAGGAGCTGGCCGGATGCGAGA[BHQ-2] |

Underlined: Tagging portion of PTO

### Example 2: Preparation of Reaction Mixtures

Using the oligonucleotides of Combinations 1 to 5 prepared in Example 1 above, reaction mixtures were prepared as follows. Taq DNA polymerase having 5′ nuclease activity was included in the reaction mixtures for extension of forward primers and reverse primers, and oligonucleotide cleavage.

### <Reaction Mixture of Combination 1>

Target nucleic acids (Tube 1: 500 pg of CT genomic DNA; Tube 2: 500 pg of NG genomic DNA; Tube 3: Mixture of 500 pg of CT genomic DNA and 500 pg of NG genomic DNA, and Tube 4: Distilled water (Negative control)) were mixed with: 5 pmole of forward primer (SEQ ID NO: 1), 5 pmole of reverse primer (SEQ ID NO: 2), 3 pmole of PTO (SEQ ID NO: 3) and 1 pmole of CTO (SEQ ID NO: 4) as oligonucleotides for the CT target nucleic acid; and 5 pmole of forward primer (SEQ ID NO: 5), 5 pmole of reverse primer (SEQ ID NO: 6), 1 pmole of PTO (SEQ ID NO: 7), and 1 pmole of CTO (SEQ ID NO: 8) as oligonucleotides for the NG target nucleic acid, and then combined with 5 μL of 4× Master mix (final, 200 μM of dNTPs, 2 mM of MgCl₂, 2U of Taq DNA polymerase) (Enzynomics, Korea), to prepare a reaction mixture in the final volume of 20 μL.

### <Reaction Mixture of Combination 2>

Target nucleic acids (Tube 1: 50 pg of CT genomic DNA; Tube 2: 50 pg of UP genomic DNA; Tube 3: Mixture of 50 pg of CT genomic DNA and 50 pg of UP genomic DNA, and Tube 4: Distilled water (Negative control)) were mixed with: 5 pmole of forward primer (SEQ ID NO: 1), 5 pmole of reverse primer (SEQ ID NO: 2), 3 pmole of PTO (SEQ ID NO: 3) and 1 pmole of CTO (SEQ ID NO: 4) as oligonucleotides for the CT target nucleic acid; and 5 pmole of forward primer (SEQ ID NO: 9), 5 pmole of reverse primer (SEQ ID NO: 10), 1 pmole of PTO (SEQ ID NO: 11), and 5 pmole of CQO (SEQ ID NO: 12) as oligonucleotides for the UP target nucleic acid, and then combined with 5 μL of 4× Master mix (final, 200 μM of dNTPs, 2 mM of MgCl₂, 2U of Taq DNA polymerase) (Enzynomics, Korea), to prepare a reaction mixture in the final volume of 20 μL.

### <Reaction Mixture of Combination 3>

Target nucleic acids (Tube 1: 500 pg of CT genomic DNA; Tube 2: 500 pg of NG genomic DNA; Tube 3: Mixture of 500 pg of CT genomic DNA and 500 pg of NG genomic DNA, and Tube 4: Distilled water (Negative control)) were mixed with: 5 pmole of forward primer (SEQ ID NO: 1), 5 pmole of reverse primer (SEQ ID NO: 2), 5 pmole of PTO (SEQ ID NO: 13) and 2 pmole of CTO (SEQ ID NO: 14) as oligonucleotides for the CT target nucleic acid; and 5 pmole of forward primer (SEQ ID NO: 5), 5 pmole of reverse primer (SEQ ID NO: 6), 3 pmole of PTO (SEQ ID NO: 7), and 1 pmole of CTO (SEQ ID NO: 8) as oligonucleotides for the NG target nucleic acid, and then combined with 5 μL of 4× Master mix (final, 200 μM of dNTPs, 2 mM of MgCl₂, 2U of Taq DNA polymerase) (Enzynomics, Korea), to prepare a reaction mixture in the final volume of 20 μL.

### <Reaction Mixture of Combination 4>

Target nucleic acids (Tube 1: 500 pg of NG genomic DNA; Tube 2: 500 pg of UP genomic DNA; Tube 3: Mixture of 500 pg of NG genomic DNA and 500 pg of UP genomic DNA, and Tube 4: Distilled water (Negative control)) were mixed with: 5 pmole of forward primer (SEQ ID NO: 5), 5 pmole of reverse primer (SEQ ID NO: 6), 1 pmole of PTO (SEQ ID NO: 7) and 1 pmole of CTO (SEQ ID NO: 8) as oligonucleotides for the NG target nucleic acid; and 5 pmole of forward primer (SEQ ID NO: 9), 5 pmole of reverse primer (SEQ ID NO: 10), 0.5 pmole of PTO (SEQ ID NO: 11), and 3 pmole of CQO (SEQ ID NO: 12) as oligonucleotides for the UP target nucleic acid, and then combined with 5 μL of 4× Master mix (final, 200 μM of dNTPs, 2 mM of MgCl₂, 2U of Taq DNA polymerase) (Enzynomics, Korea), to prepare a reaction mixture in the final volume of 20 uL.

### <Reaction Mixture of Combination 5>

Target nucleic acids (Tube 1: 50 pg of CT genomic DNA; Tube 2: 50 pg of NG genomic DNA; Tube 3: 50 pg of UP genomic DNA; Tube 4: Mixture of 50 pg of CT genomic DNA and 50 pg of NG genomic DNA; Tube 5: Mixture of 50 pg of CT genomic DNA and 50 pg of UP genomic DNA; Tube 6: Mixture of 50 pg of NG genomic DNA and 50 pg of UP genomic DNA; Tube 7: Mixture of 50 pg of CT genomic DNA, 50 pg of NG genomic DNA and 50 pg of UP genomic DNA; Tube 8: Distilled water (Negative Control)) were mixed with: 5 pmole of forward primer (SEQ ID NO: 1), 5 pmole of reverse primer (SEQ ID NO: 2), 3 pmole of PTO (SEQ ID NO: 3), and 1 pmole of CTO (SEQ ID NO: 4) as oligonucleotides for the CT target nucleic acid; 5 pmole of forward primer (SEQ ID NO: 5), 5 pmole of reverse primer (SEQ ID NO: 6), 1 pmole PTO (SEQ ID NO: 7), and 1 pmole CTO (SEQ ID NO: 8) as oligonucleotides for the NG target nucleic acid; and 5 pmole of forward primer (SEQ ID NO: 9), 5 pmole of reverse primer (SEQ ID NO: 10), 0.5 pmole of PTO (SEQ ID NO: 11), and 3 pmole of CQO (SEQ ID NO: 12) as oligonucleotides for the UP target nucleic acid, and then combined with 5 μL of 4× Master mix (final, 200 μM of dNTPs, 2 mM of MgCl₂, 2U of Taq DNA polymerase) (Enzynomics, Korea), to prepare a reaction mixture in the final volume of 20 μL.

### Example 3: Real-Time PCR

Next, a real-time PCR was performed using the reaction mixtures of Combination 1 to Combination 5, prepared in Example 2.

### <Real-Time PCR Using the Reaction Mixture of Combination 1>

A tube containing the reaction mixture of Combination 1 was placed in a real-time thermal cycler (CFX96 Real-time Cycler, Bio-Rad) and incubated at 50° C. for 4 minutes and denatured at 95° C. for 15 minutes, and then subjected to 50 repetitions of a cycle of 15 seconds at 57° C., 1 second at 50° C., 10 seconds at 72° C., 1 second at 85° C., and 10 seconds at 95° C. Detection of signals was performed at 50° C. (first detection temperature) and 72° C. (second detection temperature) at each cycle.

A signal change was measured by comparing between the cases when the target nucleic acid is present and when it is not. In this connection, on the basis of the signal value of a negative control (that is, RFU is 0), if the signal value at the first detection temperature is greater than or equal to a set threshold value of 300, the signal was considered to have changed, and if the signal value at the second detection temperature was less than or equal to a set threshold value of −300, the signal was considered to have changed.

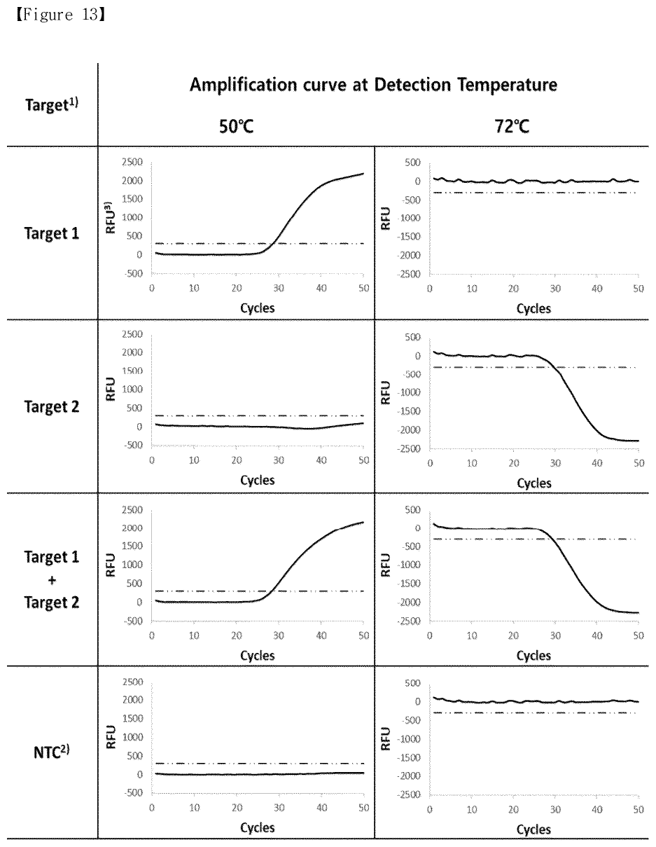

As a result, as shown in FIG. 13, in Tube 1 containing the CT target nucleic acid, a signal change was identified at the first detection temperature, and in Tube 2 containing the NG target nucleic acid, a signal change was identified at the second detection temperature, and in Tube 3 containing the CT and NG target nucleic acids, a signal change was identified at the first detection temperature and the second detection temperature, respectively.

Meanwhile, the negative control, Tube 4, provided a constant signal during amplification reactions at the first detection temperature and the second detection temperature both. That is, no signal change was identified.

TABLE 3

| | Ct (Cycle threshold) | |
|---|---|---|
| | First detection temperature | Second detection temperature |
| Tube | (50° C.) | (72° C.) |
| 1 | 28.53 | N/A |
| 2 | N/A | 29.59 |
| 3 | 28.27 | 29.28 |
| 4 | N/A | N/A |

Tube 1: 500 pg of CT genomic DNA;
Tube 2: 500 pg of NG genomic DNA;
Tube 3: Mixture of 500 pg of CT genomic DNA and 500 pg of NG genomic DNA;
Tube 4: Negative control;
N/A: Not Applicable

Also, as shown in Table 3, there was no difference between Ct (cycle threshold) value (28.53) indicative of the presence of CT in Tube 1 that contains the CT target nucleic acid only, and Ct value (28.27) indicative of the presence of CT in Tube 3 that contains both CT and NG target nucleic acids, and there was no difference between Ct value (29.59) indicative of the presence of NG in Tube 2 that contains the NG target nucleic acid only, and Ct value (29.28) indicative of the presence of NG in Tube 3 that contains both CT and NG target nucleic acids.

These results indicate that, at each detection temperature, only the signal change that is dependent on the presence of a corresponding target nucleic acid is provided, without providing signal changes dependent on the presence of the other target nucleic acids. That is, these results indicate that a plurality of different target nucleic acids can be each independently detected at its corresponding detection temperature. Accordingly, the method according to the present disclosure has advantages in that it can determine the presence of a particular target nucleic acid by a signal change alone detected at a particular detection temperature, without having to consider signal changes detected at detection temperatures other than the particular detection temperature (e.g., other detection temperatures than the particular detection temperature, that is, detection temperatures providing signal changes indicative of the presence of other target nucleic acids).

### <Real-Time PCR Using the Reaction Mixture of Combination 2>

A tube containing the reaction mixture of Combination 2 was placed in a real-time thermal cycler (CFX96 Real-time Cycler, Bio-Rad) and incubated at 50° C. for 4 minutes and denatured at 95° C. for 15 minutes, and then subjected to 50 repetitions of a cycle of 15 seconds at 57° C., 1 second at 50° C., 10 seconds at 72° C., 1 second at 85° C., and 10 seconds at 95° C. Detection of signals was performed at 57° C. (first detection temperature) and 85° C. (second detection temperature) at each cycle.

A signal change was measured by comparing between the cases when the target nucleic acid is present and when it is not. In this connection, on the basis of the signal value of a negative control (that is, RFU is 0), if the signal value at the first detection temperature and the second detection temperature is greater than or equal to a set threshold value of 300, the signal was considered to have changed.

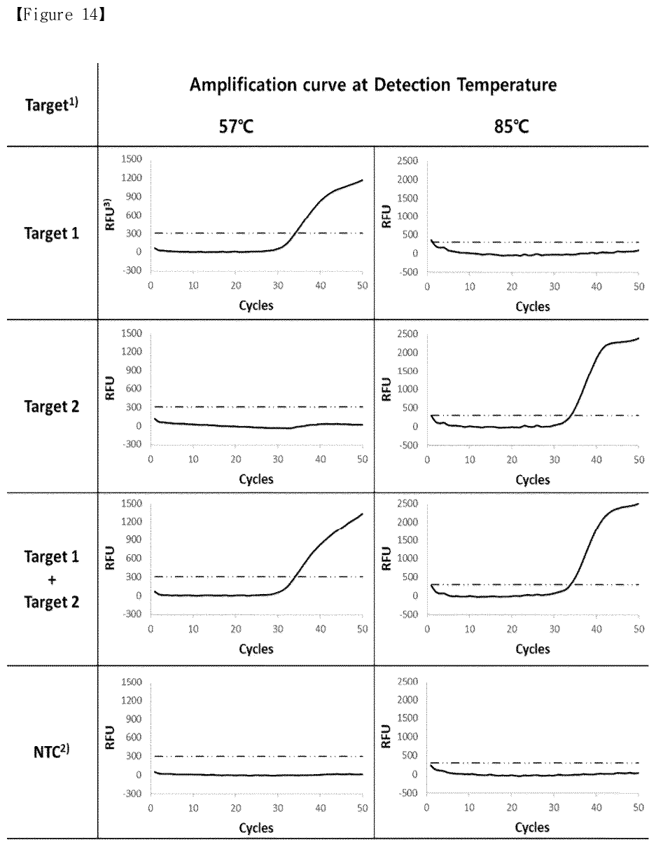

As a result, as shown in FIG. 14, in Tube 1 containing the CT target nucleic acid, a signal change was identified at the first detection temperature, and in Tube 2 containing the UP target nucleic acid, a signal change was identified at the second detection temperature, and in Tube 3 containing the CT and UP target nucleic acids, a signal change was identified at the first detection temperature and the second detection temperature, respectively.

Meanwhile, the negative control, Tube 4, showed a constant signal during amplification reactions at the first detection temperature and second detection temperature both. That is, no signal change was detected.

TABLE 4

| | Ct (Cycle threshold) | |
|---|---|---|
| | First detection temperature | Second detection temperature |
| Tube | (57° C.) | (85° C.) |
| 1 | 34.29 | N/A |
| 2 | N/A | 33.93 |
| 3 | 33.83 | 33.47 |
| 4 | N/A | N/A |

Tube 1: 50 pg of CT genomic DNA;
Tube 2: 50 pg of UP genomic DNA;
Tube 3: Mixture of 50 pg of CT genomic DNA and 50 pg of UP genomic DNA;
Tube 4: Negative control;
N/A: Not Applicable

Also, as shown in Table 4, there was no difference between Ct (cycle threshold) value (34.29) indicative of the presence of CT in Tube 1 that contains the CT target nucleic acid only, and Ct value (33.83) indicative of the presence of CT in Tube 3 containing both CT and UP target nucleic acids, and there was no difference between Ct value (33.93) indicative of the presence of UP in Tube 2 that contains the UP target nucleic acid only, and Ct value (33.47) indicative of the presence of UP in Tube 3 containing both CT and UP target nucleic acids.

These results indicate that, as described above, a plurality of different target nucleic acids can be each independently detected at their respective detection temperatures.

### <Real-Time PCR Using the Reaction Mixture of Combination 3>

A tube containing the reaction mixture of Combination 3 was placed in a real-time thermal cycler (CFX96 Real-time Cycler, Bio-Rad) and incubated at 50° C. for 4 minutes and denatured at 95° C. for 15 minutes, and then subjected to 50 repetitions of a reaction process of 15 seconds at 57° C., 1 second at 50° C., 10 seconds at 72° C., 1 second at 85° C., and 10 seconds at 95° C. Detection of signals was performed at 57° C. (first detection temperature) and 72° C. (second detection temperature) at each cycle.

A signal change was measured by comparing between the cases when the target nucleic acid is present and when it is not. In this connection, on the basis of the signal value of a negative control (that is, RFU is 0), if the signal value at the first detection temperature and the second detection temperature was less than or equal to a set threshold value of −300, the signal was considered to have changed.

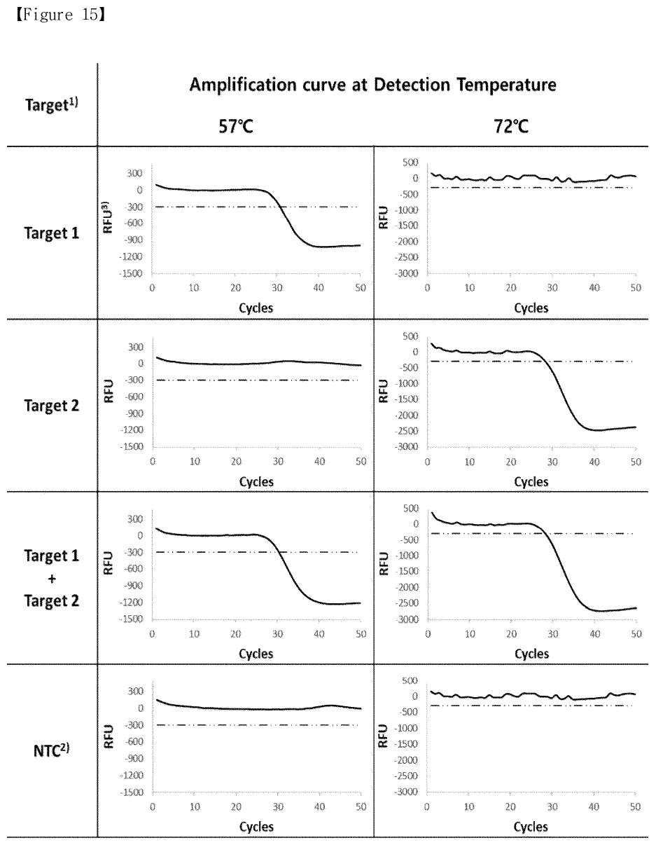

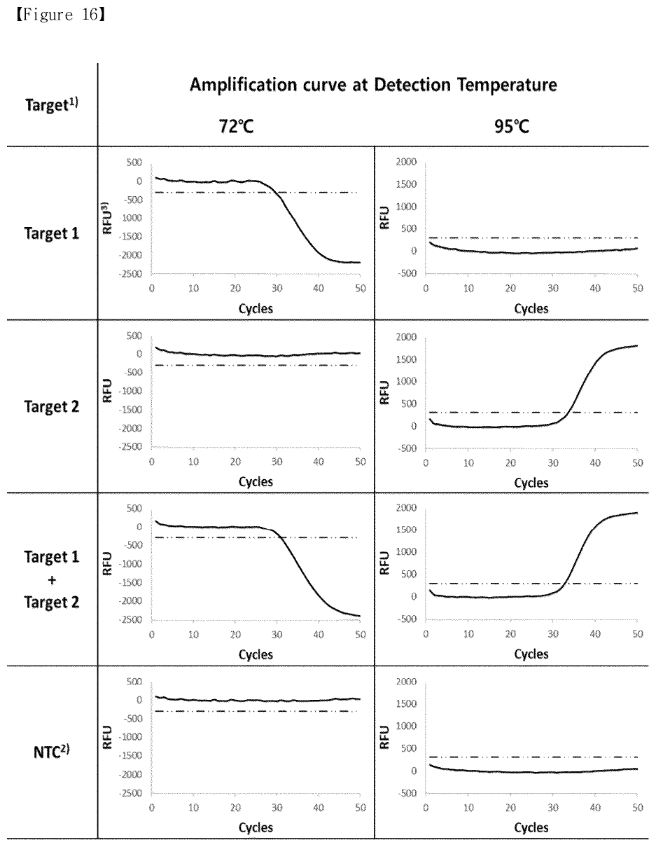

As a result, as shown in FIG. 15, in Tube 1 containing the CT target nucleic acid, a signal change was identified at the first detection temperature, and in Tube 2 containing the NG target nucleic acid, a signal change was identified at the second detection temperature, and in Tube 3 containing the CT and NG target nucleic acids, a signal change was identified at the first detection temperature and the second detection temperature, respectively.

Meanwhile, the negative control, Tube 4, showed a constant signal during amplification reactions at the first detection temperature and second detection temperature both. That is, no signal change was detected.

TABLE 5

| | Ct (Cycle threshold) | |
|---|---|---|
| | First detection temperature | Second detection temperature |
| Tube | (57° C.) | (72° C.) |
| 1 | 30.62 | N/A |
| 2 | N/A | 28.33 |
| 3 | 30.33 | 28.17 |
| 4 | N/A | N/A |

Tube 1: 500 pg of CT genomic DNA;
Tube 2: 500 pg of NG genomic DNA;
Tube 3: Mixture of 500 pg of CT genomic DNA and 500 pg of NG genomic DNA;
Tube 4: Negative control;
N/A: Not Applicable

Also as shown in Table 5 above, there was no difference between Ct (cycle threshold) value (30.62) indicative of the presence of CT in Tube 1 that contains the CT target nucleic acid only, and Ct value (30.33) indicative of the presence of CT in Tube 3 containing both CT and NG target nucleic acids, and there was no difference between Ct value (28.33) indicative of the presence of NG in Tube 2 that contains the NG target nucleic acid only, and Ct value (28.17) indicative of the presence of NG in Tube 3 containing both CT and NG target nucleic acids.

These results indicate that, as described above, a plurality of different target nucleic acids can be each independently detected at their respective detection temperatures.

### <Real-Time PCR Using the Reaction Mixture of Combination 4>

A tube containing the reaction mixture of Combination 4 was placed in a real-time thermal cycler (CFX96 Real-time Cycler, Bio-Rad) and incubated at 50° C. for 4 minutes and denatured at 95° C. for 15 minutes, and then subjected to 50 repetitions of a reaction process of 15 seconds at 57° C., 1 second at 50° C., 10 seconds at 72° C., 1 second at 85° C., and 10 seconds at 95° C. Detection of signals was performed at 72° C. (first detection temperature) and 95° C. (second detection temperature) at each cycle.

A signal change was measured by comparing between the cases when the target nucleic acid is present and when it is not. In this connection, on the basis of the signal value of a negative control (that is, RFU is 0), if the signal value at the first detection temperature is less than or equal to a set threshold value of −300, the signal was considered to have changed, and if the signal value at the second detection temperature is greater than or equal to a set threshold value of 300, the signal was considered to have changed.

As a result, as shown in FIG. 16, in Tube 1 containing the NG target nucleic acid, a signal change was identified at the first detection temperature, and in Tube 2 containing the UP target nucleic acid, a signal change was identified at the second detection temperature, and in Tube 3 containing the NG and UP target nucleic acids, a signal change was identified at the first detection temperature and the second detection temperature, respectively.

Meanwhile, the negative control, Tube 4, showed a constant signal during amplification reactions at the first detection temperature and the second detection temperature both. That is, no signal change was detected.

TABLE 6

| | Ct (Cycle threshold) | |
|---|---|---|
| | First detection temperature | Second detection temperature |
| Tube | (72° C.) | (95° C.) |
| 1 | 29.62 | N/A |
| 2 | N/A | 33.42 |
| 3 | 30.76 | 32.68 |
| 4 | N/A | N/A |

Tube 1: 500 pg of NG genomic DNA;
Tube 2: 500 pg of UP genomic DNA;
Tube 3: Mixture of 500 pg of NG genomic DNA and 500 pg of UP genomic DNA;
Tube 4: Negative control;
N/A: Not Applicable

Also, as shown in Table 6, there was no difference between Ct (cycle threshold) value (29.62) indicative of the presence of NG in Tube 1 that contains the NG target nucleic acid only, and Ct value (30.72) indicative of the presence of NG in Tube 3 that contains both NG and UP target nucleic acids, and there was no difference between Ct value (33.42) indicative of the presence of UP in Tube 2 that contains the UP target nucleic acid only, and Ct value (32.68) indicative of the presence of UP in Tube 3 containing both NG and UP target nucleic acids.

These results indicate that, as described above, a plurality of different target nucleic acid sequences can be each independently detected at their respective detection temperatures.

### <Real-Time PCR Using the Reaction Mixture of Combination 5>

A tube containing the reaction mixture of Combination 5 was placed in a real-time thermal cycler (CFX96 Real-time Cycler, Bio-Rad) and incubated at 50° C. for 4 minutes and denatured at 95° C. for 15 minutes, and then subjected to 50 repetitions of a reaction process of 15 seconds at 57° C., 1 second at 50° C., 10 seconds at 72° C., 1 second at 85° C., and 10 seconds at 95° C. Detection of signals was performed at 50° C. (first detection temperature), 72° C. (second detection temperature), and 95° C. (third detection temperature) at each cycle.

A signal change was measured by comparing between the cases when the target nucleic acid is present and when it is not. In this connection, on the basis of the signal value of a negative control (that is, RFU is 0), if the signal value at the first detection temperature and the third detection temperature was greater than or equal to a set threshold value of 300, the signal was considered to have changed, and if the signal value at the second detection temperature was less than or equal to a set threshold value of −300, the signal was considered to have changed.

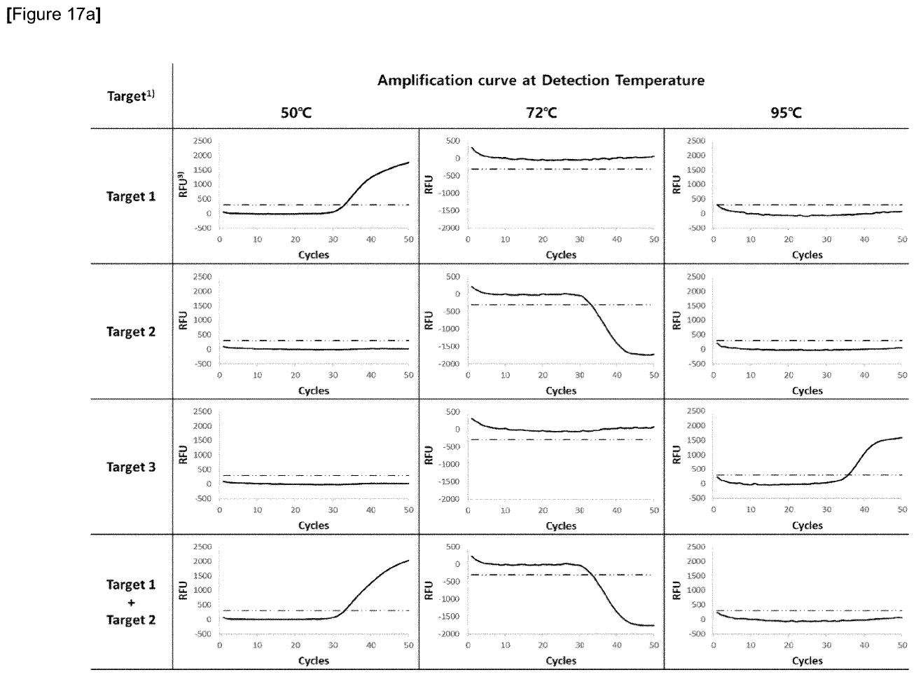

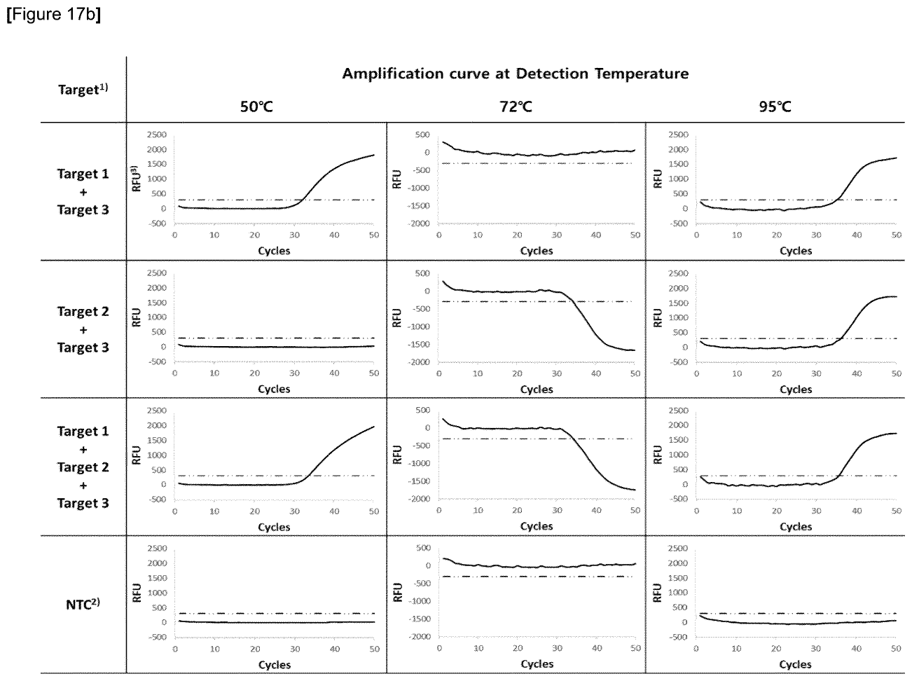

As a result, as shown in FIG. 17a and FIG. 17b, in Tube 1 containing the CT target nucleic acid, a signal change was identified at the first detection temperature, and in Tube 2 that contains the NG target nucleic acid, a signal change was identified at the second detection temperature, and in Tube 3 containing the UP target nucleic acid, a signal change was identified at the third detection temperature.

Also, in Tube 4 to Tube 7, each containing at least two of CT, NG, and UP, a signal change was identified at a corresponding detection temperature depending on target nucleic acids present in the tube.

Meanwhile, the negative control, Tube 8, showed a constant signal during amplification reactions at all of the first, second and third detection temperatures. That is, no signal change was detected.

TABLE 7

| | Ct (Cycle threshold) | | |
|---|---|---|---|
| | First detection | Second detection | Third detection |
| Tube | temperature (50° C.) | temperature (72° C.) | temperature (95° C.) |
| 1 | 32.92 | N/A | N/A |
| 2 | N/A | 33.00 | N/A |
| 3 | N/A | N/A | 35.46 |
| 4 | 33.03 | 33.23 | N/A |
| 5 | 32.47 | N/A | 34.99 |
| 6 | N/A | 33.99 | 35.91 |
| 7 | 33.34 | 34.12 | 35.32 |
| 8 | N/A | N/A | N/A |

Tube 1: 50 pg of CT genomic DNA;
Tube 2: 50 pg of NG genomic DNA;
Tube 3: 50 pg of UO genomic DNA;
Tube 4: Mixture of 50 pg of CT genomic DNA and 50 pg of NG genomic DNA;
Tube 5: Mixture of 50 pg of CT genomic DNA and 50 pg of UP genomic DNA;
Tube 6: Mixture of 50 pg of NG genomic DNA and 50 pg of UP genomic DNA;
Tube 7: Mixture of 50 pg of CT genomic DNA, 50 pg of NG genomic DNA, and 50 pg of UP genomic DNA;
Tube 8: Negative control;
N/A: Not Applicable

Also, as shown in Table 7 above, there was no difference in Ct value when a particular target nucleic acid was present alone and when it was present along with other target nucleic acids (CT: 32.92 vs 33.03 vs 32.47 vs 33.34; NG: 33.00 vs 33.23 vs 33.99 vs 34.12; UP: 35.46 vs 34.99 vs 35.91 vs 35.32).

These results indicate that, as described above, a plurality of different target nucleic acids can be each independently detected at their respective detection temperatures.

Meanwhile, as described above in the present application, a detection temperature may be selected from within the signal-changing temperature range of the composition for detecting a target nucleic acid. Further, the detection temperature may be selected in view of the signal-changing temperature ranges of other compositions for detecting target nucleic acids used together.

To confirm that a detection temperature can be selected at various points within a signal-changing temperature range, the present inventors set a different detection temperature for CT in Combination 2 and Combination 5, while using the same composition as the composition for detecting CT. That is, the detection temperature for CT of Combination 2 was set to 57° C., and the detection temperature for CT of Combination 5 was set to 50° C. As a result, it was found that Combination 2 and Combination 5 both provide a signal change indicative of the presence of the CT target nucleic acid, only at their respective detection temperatures (that is, 57° C. and 50° C.). Also, in Combination 2 and Combination 5, the detection temperature for UP was set differently while using the same composition as the composition for detecting UP. That is, the detection temperature for UP of Combination 2 was set to 85° C., and the detection temperature for UP of Combination 5 was set to 95° C. As a result, it was also found that the signal change indicative of the presence of the UP target nucleic acid was provided only at the respective corresponding detection temperatures (85° C. and 95° C.).

In addition, as described above in this specification, the signal-changing temperature range and signal-constant temperature range can be controlled by adjusting the length and/or sequence of a duplex (e.g., adjusting the Tm of the duplex) provided by the composition for detecting a target nucleic acid.

To confirm that the signal-changing temperature range and signal-constant temperature range of the composition for detecting a target nucleic acid can be controlled by adjusting the Tm of a duplex, the present inventors used the InterSC compositions with the same signal generation mechanism for different target nucleic acids (CT and NG) in Combination 3, and designed the sequences of oligonucleotides such that the CT provides a duplex with a relatively low Tm, and the NG provides a duplex with a relatively high Tm, so that even with the compositions with the same signal generation mechanism, detection can be made at different detection temperatures (that is, first detection temperature and second detection temperature).

As a result, as shown in the real-time PCR result of Combination 3, it was found that each detection temperature provides only the signal change indicative of their corresponding target nucleic acid.

Therefore, according to the present disclosure, multiple target nucleic acids can be detected in a real-time manner using a single type of label, by applying at least one of the above-described three types of signal-generation mechanisms and adjusting the signal-changing temperature range such that only the signal indicative of the presence of a single target nucleic acid is provided at each detection temperature.

Having described a preferred embodiment of the present invention, it is to be understood that variants and modifications thereof falling within the spirit of the invention may become apparent to those skilled in this art. Therefore, the scope of the present invention shall be determined by the appended claims and their equivalents.

1. A method for detecting n target nucleic acids in a sample, comprising:
(a) detecting signals at n detection temperatures, while incubating with n compositions for detecting the target nucleic acids, a sample suspected of containing at least one of the n target nucleic acids in a reaction vessel;
wherein n is an integer of 2 or more,
wherein the incubation comprises a plurality of cycles and the detection of signals is carried out at at least one of the plurality of cycles,
wherein each of the n compositions for detecting the target nucleic acids provides a signal change at a corresponding detection temperature among the n detection temperatures in the presence of a corresponding target nucleic acid, the signal change indicating the presence of a corresponding target nucleic acid,
wherein a composition for detecting an i^{th} target nucleic acid among the n compositions for detecting the target nucleic acids provides a signal change at an i^{th} detection temperature among the n detection temperatures and provides a constant signal at the other detection temperatures in the presence of the i^{th} target nucleic acid, the signal change indicating the presence of the i^{th} target nucleic acid, and
wherein i represents an integer from 1 to n, and the i^{th} detection temperature is lower than the (i+1)^{th} detection temperature; and
(b) determining the presence of the n target nucleic acids from the signals detected in step (a), wherein the presence of the i^{th} target nucleic acid is determined by the signal change detected at the i^{th} detection temperature.

2. The method of claim 1, wherein in the temperature range covering all of the n detection temperatures, the composition for detecting the i^{th} target nucleic acid has a signal-changing temperature range (SChTR) in which the signal changes depending on the presence of the i^{th} target nucleic acid, and one or two signal-constant temperature ranges (SCoTRs) in which the signal is constant even in the presence of the i^{th} target nucleic acid.

3. (canceled)

4. The method of claim 2, wherein the composition for detecting the i^{th} target nucleic acid is any one of:
(i) an Under-Signal-Change (UnderSC) composition having a characteristic that the signal-changing temperature range is lower than the signal-constant temperature range;
(ii) an Over-Signal-Change (OverSC) composition having a characteristic that the signal-changing temperature range is higher than the signal-constant temperature range; and
(iii) an Inter-Signal-Change (InterSC) composition having a characteristic that the signal-changing temperature range is higher than one of two signal-constant temperature ranges, and lower than the other of the two signal-constant temperature ranges.

5. The method of claim 2, wherein the i^{th} detection temperature is selected within the signal-changing temperature range of the composition for detecting the i^{th} target nucleic acid, wherein the i^{th} detection temperature is not included in the signal-changing temperature ranges of the compositions for detecting the other target nucleic acids.

6. The method of claim 2, wherein the signal-changing temperature range of the composition for detecting the i^{th} target nucleic acid overlaps partially with the signal-changing temperature range of a composition for detecting a target nucleic acid having an adjacent detection temperature, and does not overlap with the signal-changing temperature range of a composition for detecting a target nucleic acid having a detection temperature that is not adjacent thereto.

7. The method of claim 4, wherein, (i) when n is 2, the composition for detecting the first target nucleic acid is an UnderSC composition or an InterSC composition, and the composition for detecting the second target nucleic acid is an InterSC composition or an OverSC composition or (ii) when n is 3 or more, the composition for detecting the first target nucleic acid is an UnderSC composition or an InterSC composition, the composition for detecting the n^{th} target nucleic acid is an InterSC composition or an OverSC composition, and each of compositions for detecting target nucleic acids other than the first target nucleic acid and the n^{th} target nucleic acid is an InterSC composition.

8. (canceled)

9. The method of claim 1, wherein the composition for detecting the i^{th} target nucleic acid comprises a label that provides a signal dependent on the presence of the i^{th} target nucleic acid.

10. The method of claim 9, wherein the label is linked to an oligonucleotide or is incorporated into an oligonucleotide during the incubation.

11. The method of claim 1, wherein the composition for detecting the i^{th} target nucleic acid provides a duplex providing a signal change; wherein the duplex providing the signal change comprises a label.

12. The method of claim 9, wherein the composition for detecting the i^{th} target nucleic acid provides a duplex providing a signal change, and the composition for detecting the i^{th} target nucleic acid provides a signal from the label when the duplex providing the signal change is in an associated form or in a dissociated form.

13. (canceled)

14. The method of claim 11, wherein the duplex providing the signal change has initially been included in the composition for detecting the i^{th} target nucleic acid.

15. The method of claim 14, wherein the duplex providing the signal change is generated by hybridization between a label-linked oligonucleotide and an oligonucleotide hybridizable with the label-linked oligonucleotide.

16. The method of claim 11, wherein the duplex providing the signal change is generated in incubating.

17. The method of claim 16, wherein the duplex providing the signal change is generated by (i) hybridization between a label-linked oligonucleotide and the target nucleic acid or (ii) a cleavage reaction dependent on the presence of a target nucleic acid.

18. (canceled)

19. The method of claim 17, wherein the composition for detecting the target nucleic acid comprises a tagging oligonucleotide that hybridizes to the target nucleic acid, and the cleavage reaction dependent on the presence of the target nucleic acid involves cleavage of the tagging oligonucleotide.

20. The method of claim 11, wherein the duplex providing the signal change is a single-typed duplex or plural-typed duplexes.

21. The method of claim 20, wherein, (i) when the duplex providing the signal change is the single-typed duplex, the amount of the single-typed duplex changes depending on the presence of the target nucleic acid, thereby changing the signal or (ii) when the duplex providing the signal change is the plural-typed duplexes, the amount ratio between the plural-typed duplexes changes depending on the presence of the target nucleic acid, thereby changing the signal.

22. (canceled)

23. The method of claim 20, wherein, when the duplex is the plural-typed duplexes, the Tm values of the duplexes are different from each other.

24.-25. (canceled)

26. The method of claim 5, wherein the composition for detecting the i^{th} target nucleic acid provides a duplex providing a signal change, and the signal-changing temperature range of the composition for detecting the i^{th} target nucleic acid is determined depending on the length and/or sequence of the duplex.

27. The method of claim 1, wherein the detection of signals is carried out at at least two of the plurality of cycles.

28. The method of claim 27, wherein the signal change is measured using the signals detected at the at least two of the plurality of cycles.

29. The method of claim 1, wherein the signal change at the i^{th} detection temperature is measured using a signal detected at the at least one of the plurality of cycles and a reference signal value.

30. The method of claim 29, wherein the reference signal value is obtained from a reaction in the absence of the i^{th} target nucleic acid.

31. The method of claim 1, wherein the detection of a signal at each of the n detection temperatures is carried out using a single type of detector.

32. The method of claim 31, wherein the signals detected at the n detection temperatures are not differentiated from each other by the single type of detector.

33. The method of claim 1, wherein the incubation comprises a nucleic acid amplification reaction.

34. (canceled)

35. A method for detecting two target nucleic acids in a sample, comprising:
(a) detecting signals at a first detection temperature and a second detection temperature, while incubating the sample suspected of containing at least one of the two target nucleic acids with a composition for detecting a first target nucleic acid and a composition for detecting a second target nucleic acid in a reaction vessel;
wherein the incubation comprises a plurality of cycles, and the detection of signals is carried out at at least one of the plurality of cycles,
wherein the composition for detecting the first target nucleic acid provides a signal change at the first detection temperature and provides a constant signal at the second detection temperature in the presence of the first target nucleic acid, the signal change indicating the presence of the first target nucleic acid; and the composition for detecting the second target nucleic acid provides a signal change at the second detection and provides a constant signal at the first detection temperature in the presence of the second target nucleic acid, the signal change indicating the presence of the second target nucleic acid, and
wherein the first detection temperature is lower than the second detection temperature, and
(b) determining the presence of the two target nucleic acids from the signals detected in step (a); wherein the presence of the first target nucleic acid is determined by the signal change detected at the first detection temperature, and the presence of the second target nucleic acid is determined by the signal change detected at the second detection temperature.

36. A method for detecting three target nucleic acids in a sample, comprising:
(a) detecting signals at a first detection temperature, a second detection temperature, and a third detection temperature, while incubating the sample suspected of containing at least one of the three target nucleic acids with a composition for detecting a first target nucleic acid, a composition for detecting a second target nucleic acid, and a composition for detecting a third target nucleic acid in a reaction vessel;
wherein the incubation comprises a plurality of cycles, and the detection of signals is carried out at at least one of the plurality of cycles,
wherein the composition for detecting the first target nucleic acid provides a signal change at the first detection temperature and provides a constant signal at the second detection temperature and the third detection temperature in the presence of the first target nucleic acid, the signal change indicating the presence of the first target nucleic acid; the composition for detecting the second target nucleic acid provides a signal change at the second detection temperature and provides a constant signal at the first detection temperature and the third detection temperature in the presence of the second target nucleic acid, the signal change indicating the presence of the second target nucleic acid; and the composition for detecting the third target nucleic acid provides a signal change at the third detection temperature and provides a constant signal at the first detection temperature and the second detection temperature in the presence of the third target nucleic acid, the signal change indicating the presence of the third target nucleic acid, and
wherein the first detection temperature is lower than the second detection temperature, and the second detection temperature is lower than the third detection temperature, and
(b) determining the presence of the three target nucleic acids from the signals detected in step (a),
wherein the presence of the first target nucleic acid is determined by the signal change detected at the first detection temperature, the presence of the second target nucleic acid is determined by the signal change detected at the second detection temperature, and the presence of the third target nucleic acid is determined by the signal change detected at the third detection temperature.

37-51. (canceled)


SEQUENCE LISTING (14 sequences)

SEQ ID NO: 1 - DNA, 25 bp, Artificial Sequence
  CT-Forward primer
gagttttaaaatgggaaattctggt

SEQ ID NO: 2 - DNA, 25 bp, Artificial Sequence
  CT-Reverse primer
ccaattgtaatagaagcattggttg

SEQ ID NO: 3 - DNA, 39 bp, Artificial Sequence
  CT-PTO
gattacgcgaccgcatcagaagctgtcattttggctgcg

SEQ ID NO: 4 - DNA, 39 bp, Artificial Sequence
  CT-CTO
tattttatttttattttttttatgtgcggtcgcgtaatc

SEQ ID NO: 5 - DNA, 21 bp, Artificial Sequence
  NG-Forward primer
caatggatcggtatcactcgc

SEQ ID NO: 6 - DNA, 20 bp, Artificial Sequence
  NG-Reverse primer
tacgcctgctactttcacgc

SEQ ID NO: 7 - DNA, 35 bp, Artificial Sequence
  NG-PTO
gtacgcgataccgggcccctcattggcgtgtttcg

SEQ ID NO: 8 - DNA, 40 bp, Artificial Sequence
  NG-CTO
ggctcgactgtcgtctctgctcatgcccggtatcgcgtac

SEQ ID NO: 9 - DNA, 27 bp, Artificial Sequence
  UP-Forward primer
  misc_feature (9): n denotes deoxyinosine
  misc_feature (23): n denotes deoxyinosine
cagatttanacggtattacaacnggtg

SEQ ID NO: 10 - DNA, 32 bp, Artificial Sequence
  UP-Reverse primer
  misc_feature (12): n denotes deoxyinosine
  misc_feature (29): n denotes deoxyinosine
ttacttcagcantacctaatttatcatcnaac

SEQ ID NO: 11 - DNA, 52 bp, Artificial Sequence
  UP-PTO
cacgtagggtttttccaccaccattctcgcatccggccagctcctctccagg

SEQ ID NO: 12 - DNA, 28 bp, Artificial Sequence
  UP-CQO
cctggagaggagctggccggatgcgaga

SEQ ID NO: 13 - DNA, 37 bp, Artificial Sequence
  CT-PTO
catggccaaggcatcagaagctgtcattttggctgcg

SEQ ID NO: 14 - DNA, 32 bp, Artificial Sequence
  CT-CTO
ttttttttatttttttttatgccttggccatg